(19) United States
(12) Patent Application Publication
(10) Pub. No.: US 2024/0382429 A1
(43) Pub. Date: Nov. 21, 2024

(54) POLYMERIC NANOPARTICLE GENETIC VACCINES

(71) Applicant: The Johns Hopkins University, Baltimore, MD (US)

(72) Inventors: Jordan J. GREEN, Woodstock, MD (US); Elana BEN-AKIVA, Baltimore, MD (US); Johan KARLSSON, Baltimore, MD (US); Stephany Yi TZENG, Baltimore, MD (US); Kelly RHODES, Baltimore, MD (US); Sarah Y. NESHAT, Baltimore, MD (US)

(21) Appl. No.: 18/693,984

(22) PCT Filed: Sep. 28, 2022
(86) PCT No.: PCT/US2022/077173

§ 371 Date: Mar. 21, 2024

Related U.S. Application Data
(60) Provisional application No. 63/249,349, filed on Sep. 28, 2021.

(51) Int. Cl. A61K 9/50; A61K 39/00; A61P 37/04; C12N 9/12

(52) U.S. Cl. CPC A61K 9/5015; A61K 39/001154; A61P 37/04; C12N 9/1241; A61K 2039/53; A61K 2039/55555; A61K 2039/55561

(57) ABSTRACT

Compositions comprising degradable polymers combined with nucleic acids, such as DNA and RNA, encoding antigen and their use as genetic vaccines are disclosed.

### STATEMENT OF GOVERNMENTAL INTEREST

This invention was made with government support under grant EB028239 awarded by the National Institutes of Health. The government has certain rights in the invention.

### BACKGROUND

Genetic vaccines using mRNA to encode a pathogenic antigen are one of the most promising advancements in vaccine technology. There is a need, however, for more efficient transfection and targeting of antigen-presenting cells (APCs) to evoke stronger cellular immune responses against infectious diseases including, but not limited to COVID-19, cancers, and autoimmune diseases.

### SUMMARY

In some aspects, the presently disclosed subject matter provides a composition comprising a polymer of formula (I) and one or more nucleic acids encoding antigen:

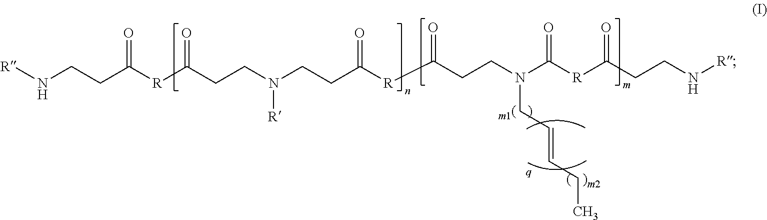

wherein m and n are each independently an integer from 1 to 10,000; m1 is an integer selected from 0, 1, 2, 3, 4, 5, 6, 7, 8, 9, and 10; m2 is an integer selected from the group consisting of 1, 2, 3, 4, 5, 6, 7, 8, 9, 10, 11, 12, 13, 14, 15, 16, 17, 18, 19, and 20; and q is an integer selected from 0 or 1;
wherein —(CH₂)ₘ₁—(C═C)_{q}—(CH₂)ₘ₂—CH₃ comprises a hydrophobic sidechain;
R comprises a divalent radical comprising a biodegradable ester linkage and/or a bioreducible disulfide linkage;
R′ is hydrophilic sidechain comprising a monovalent radical derived from a hydrophilic amine monomer;
R″ is monovalent radical derived from an amine-containing end capping group; and pharmaceutically acceptable salts thereof.

In some aspects, R is selected from the group consisting of:

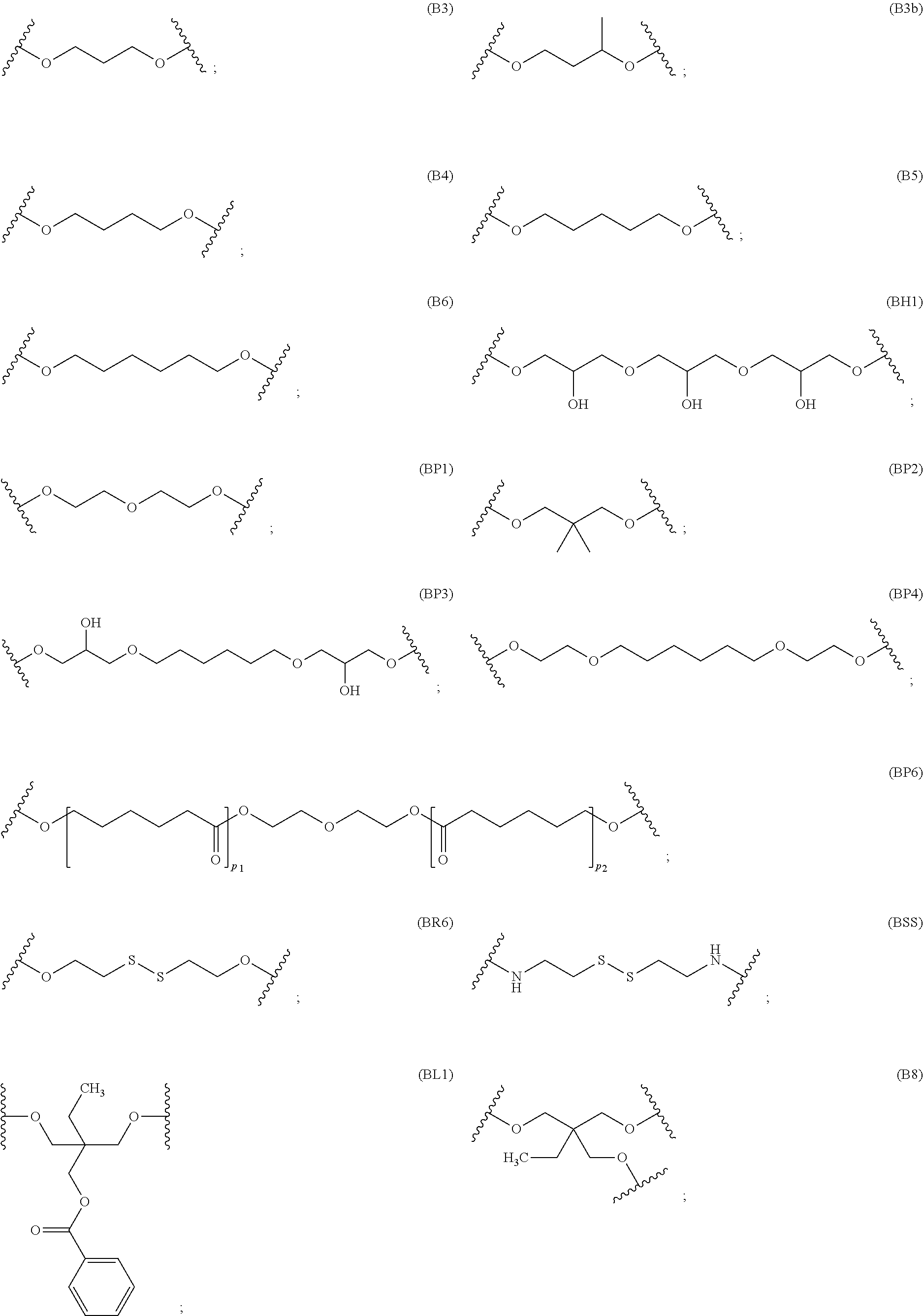

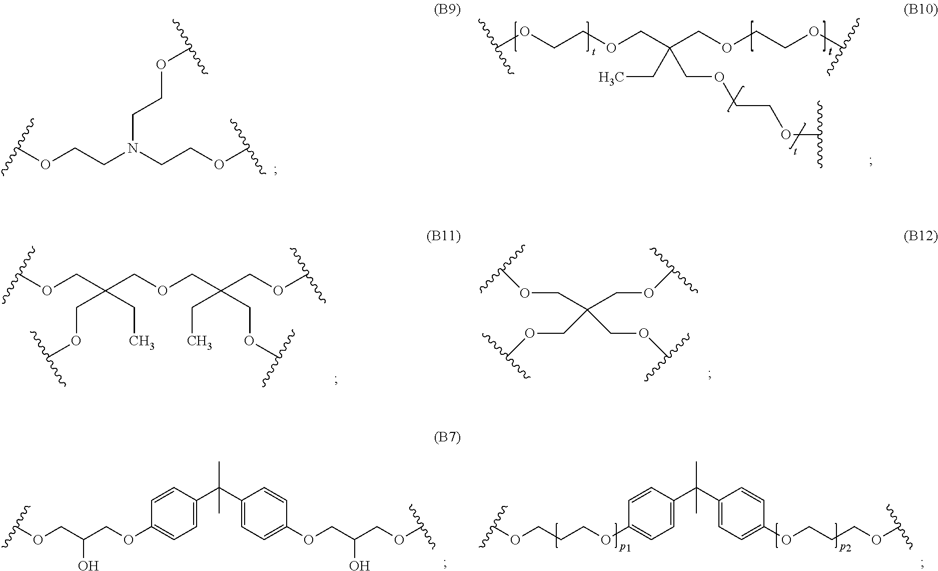

wherein each p1, p2, and t is independently an integer selected from the group consisting of 1, 2, 3, 4, 5, 6, 7, 8, 9, and 10.

In some aspects, R′ is selected from the group consisting of:

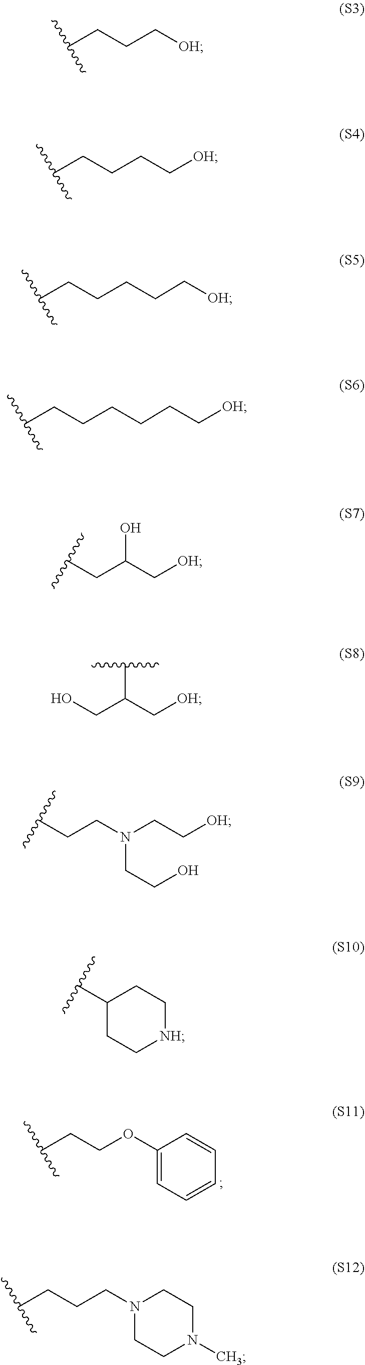

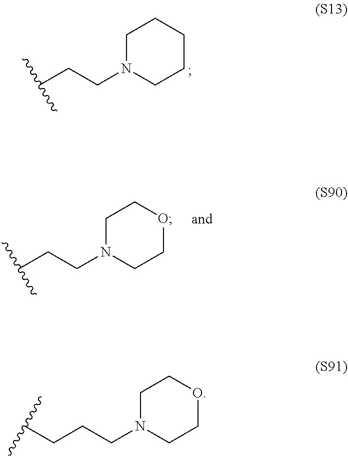 [SMILES: *CCN1CCCCC1 ; *CCN1CCOCC1 ; "and" ; *CCCN1CCOCC1]

In some aspects, R″ is selected from the group consisting of:

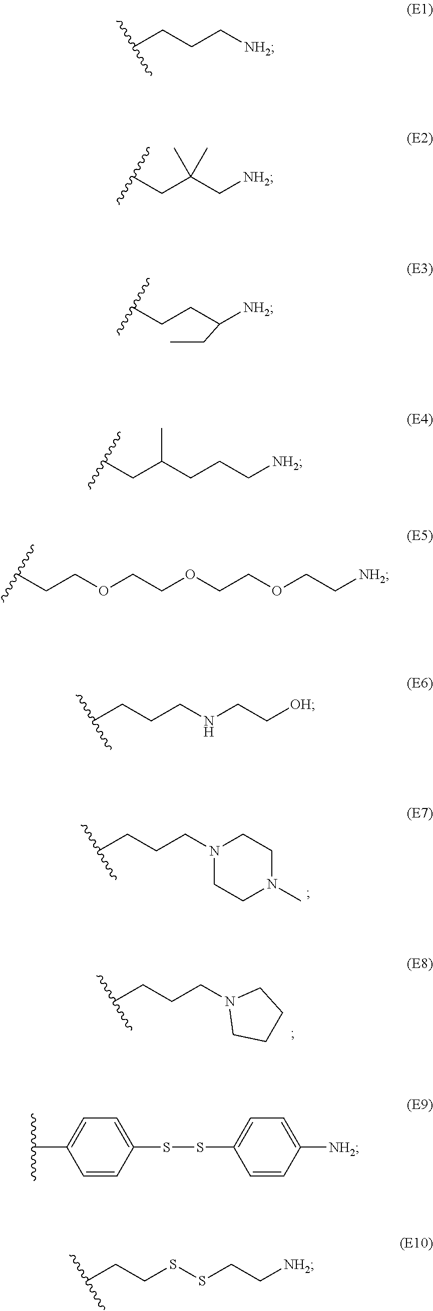

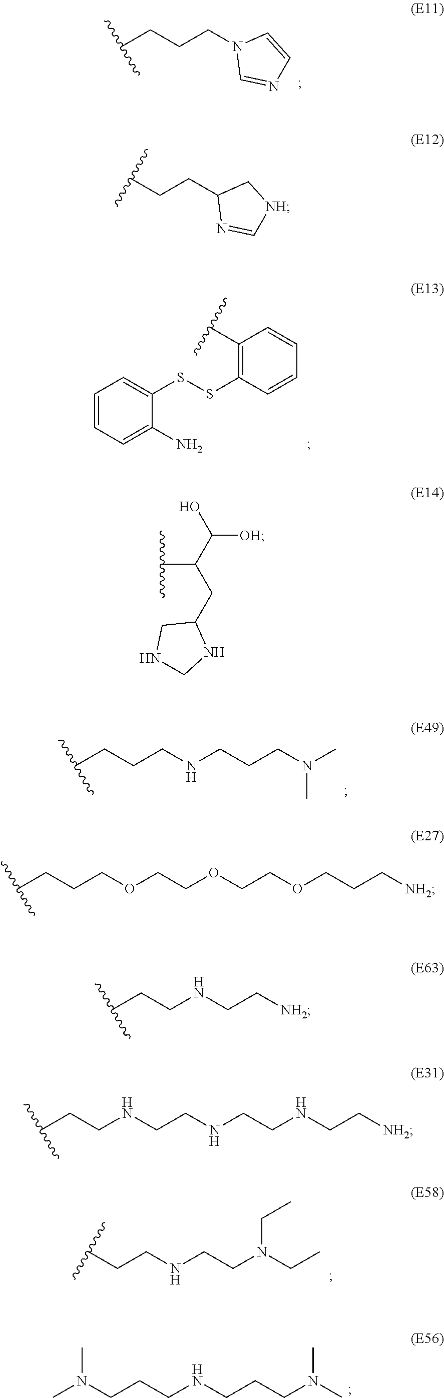

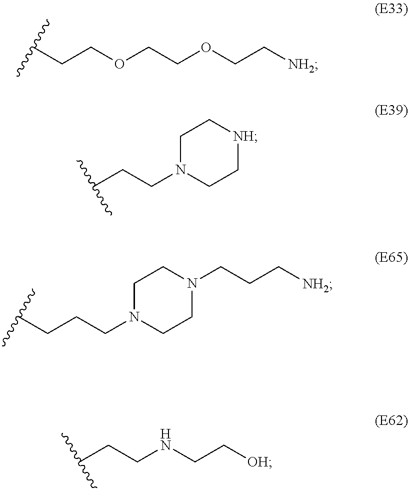 [SMILES: *CCOCCOCCN ; *CCN1CCNCC1 ; *CCCN1CCN(CCCN)CC1 ; *CCNCCO]

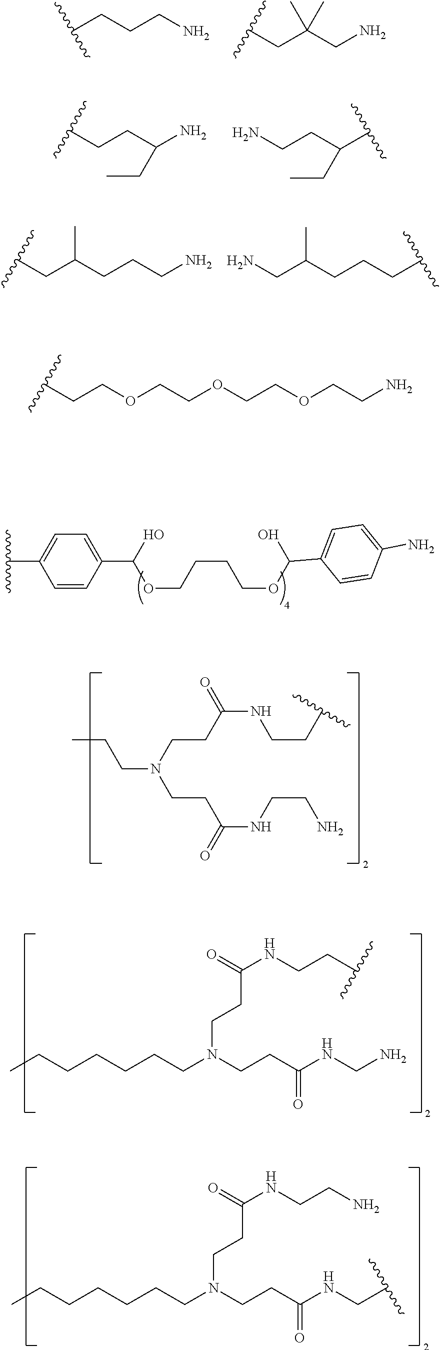

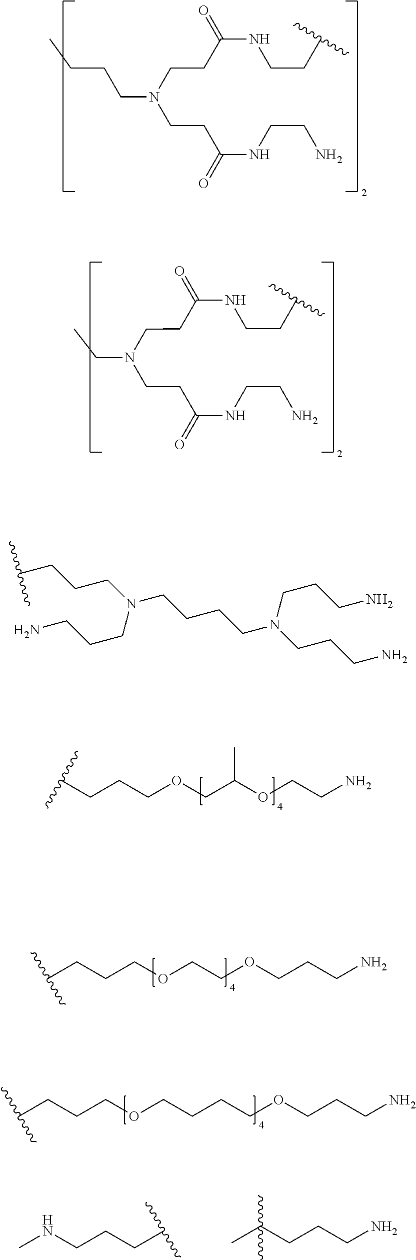

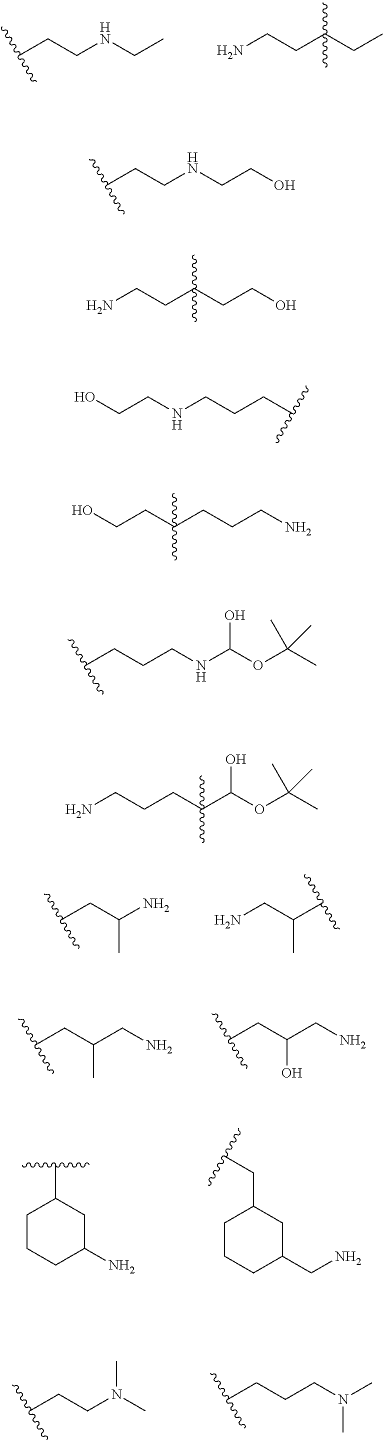

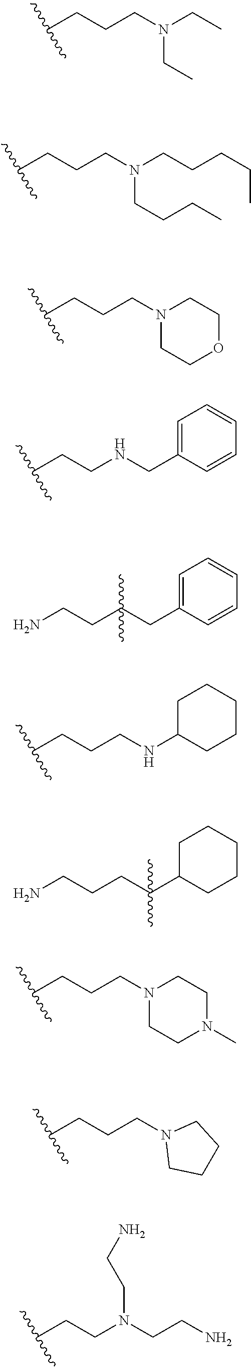

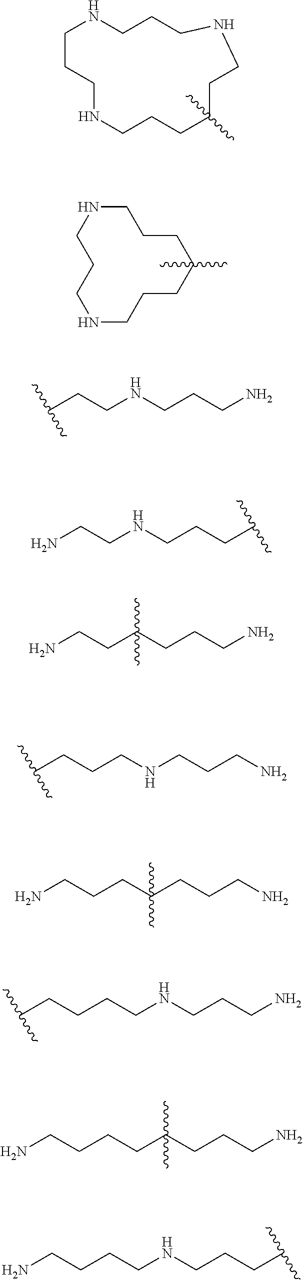

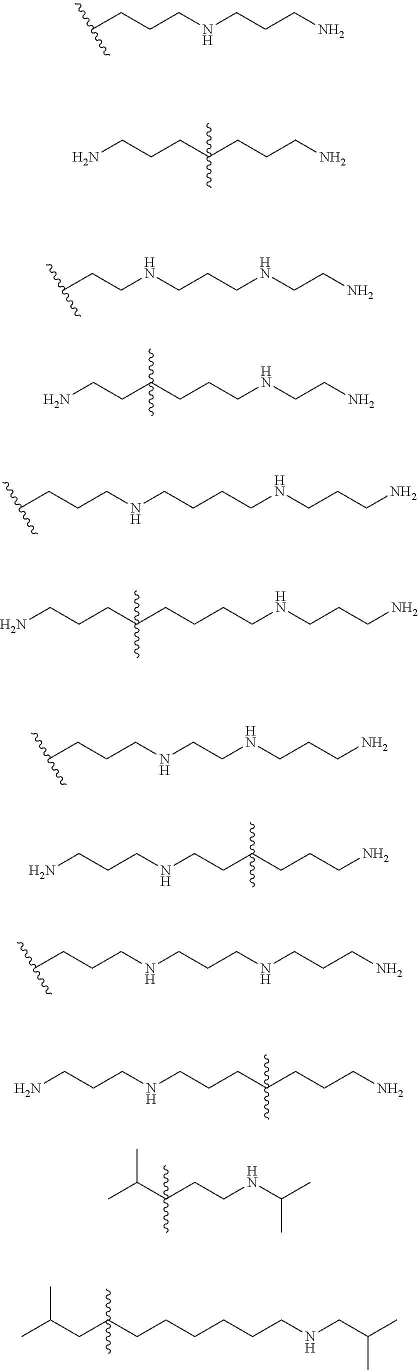

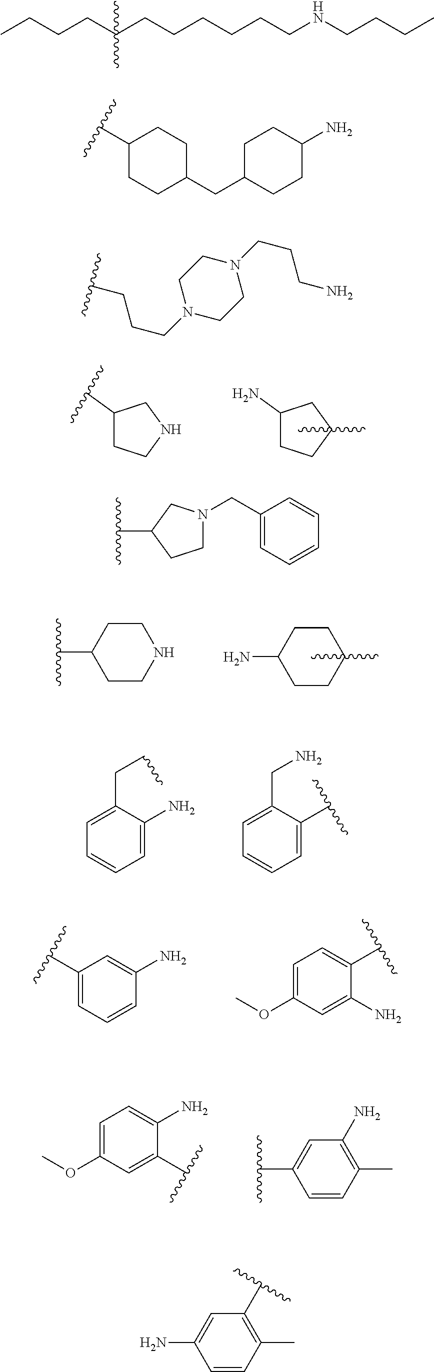

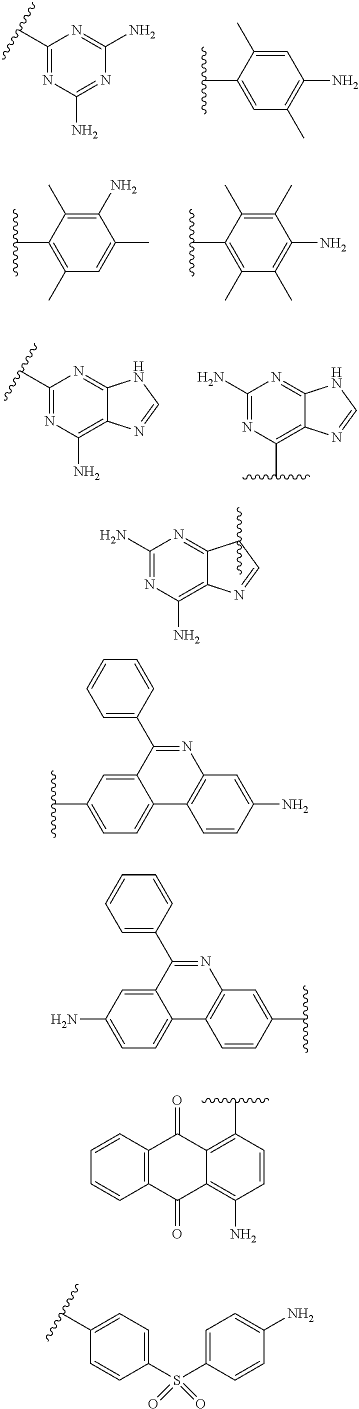

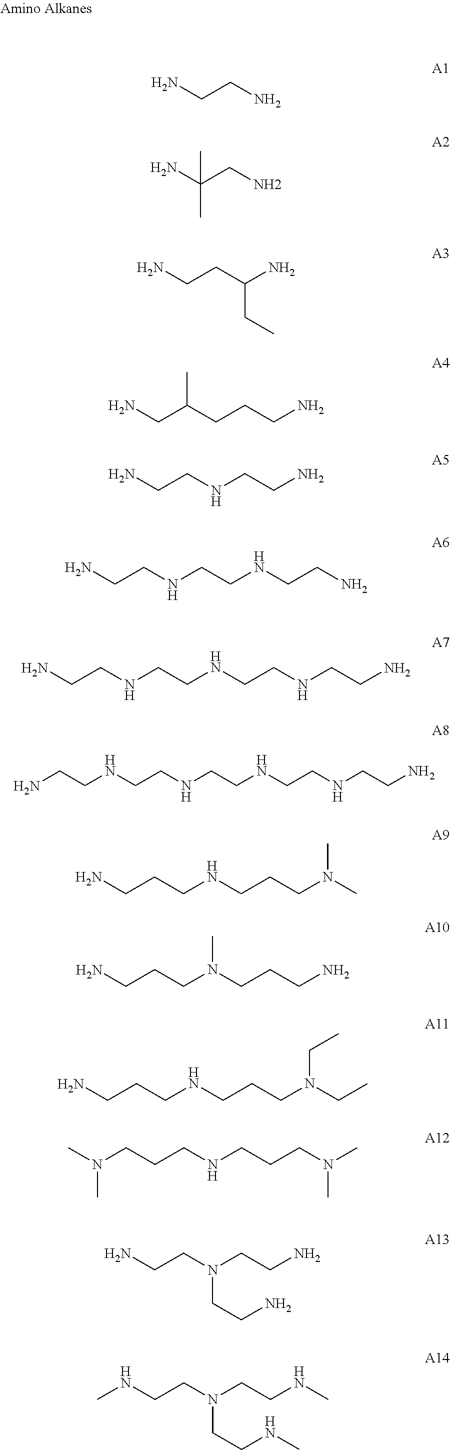

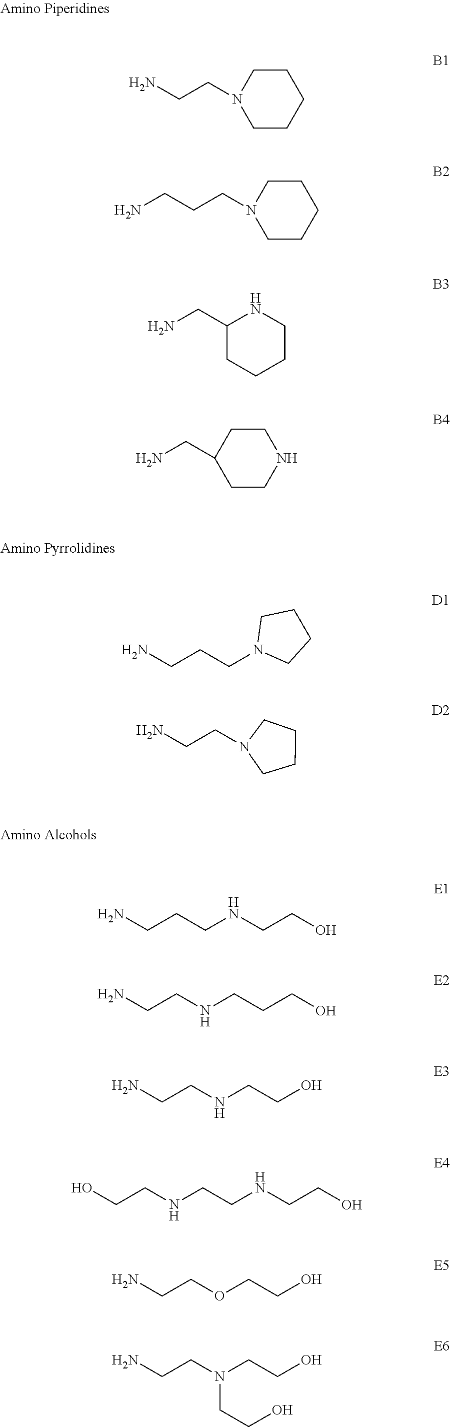

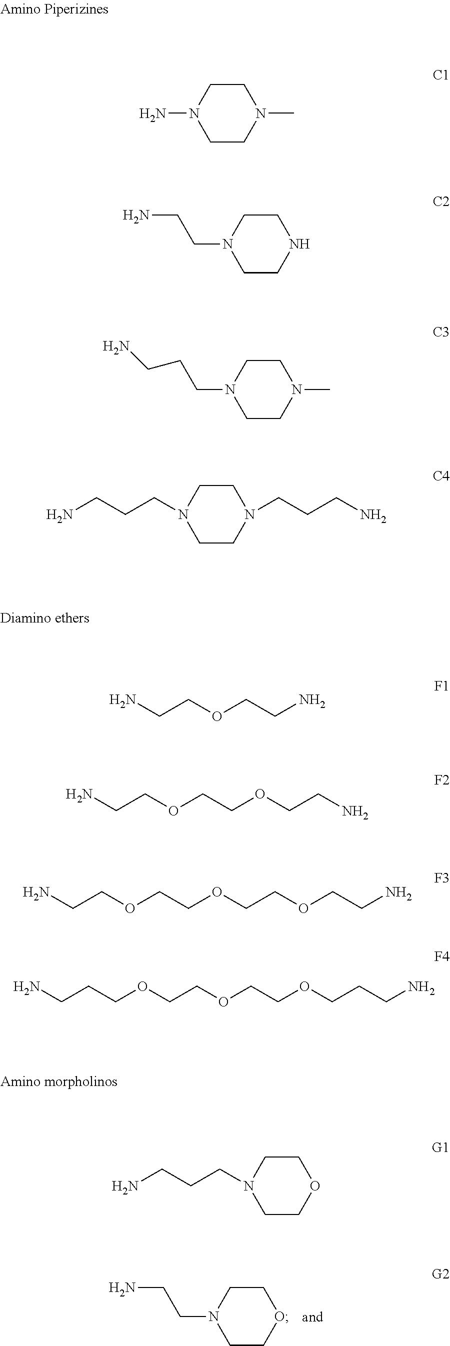

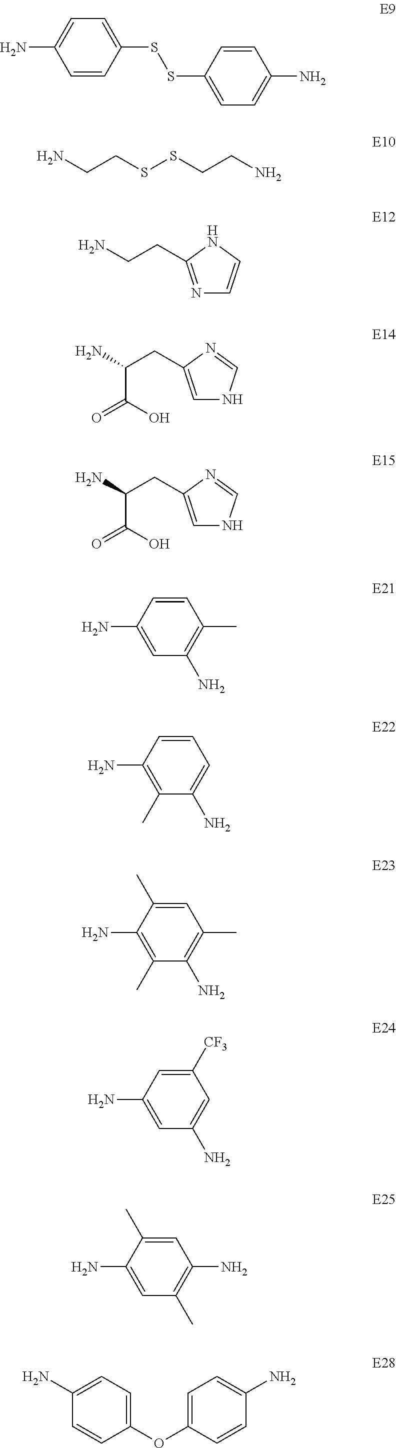

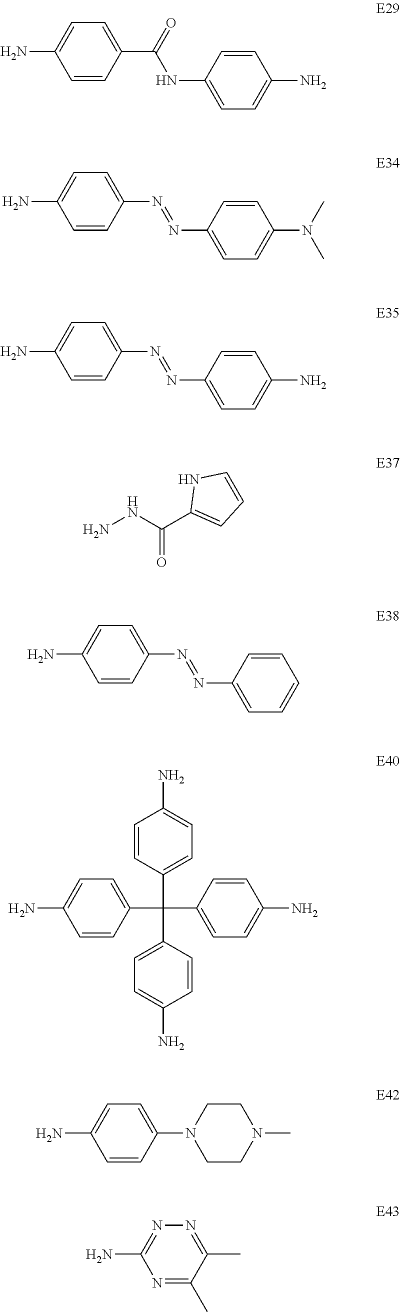

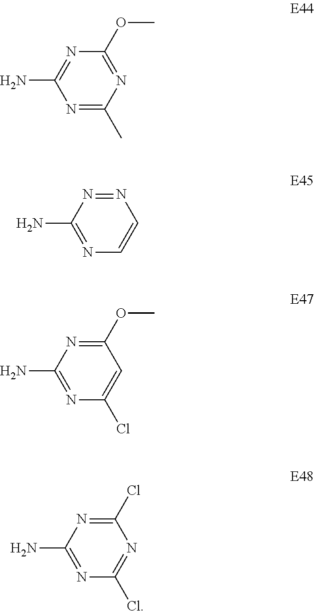

In some aspects, —(CH₂)ₘ₁—(C═C)_{q}—(CH₂)ₘ₂—CH₃ is selected from the group consisting of:

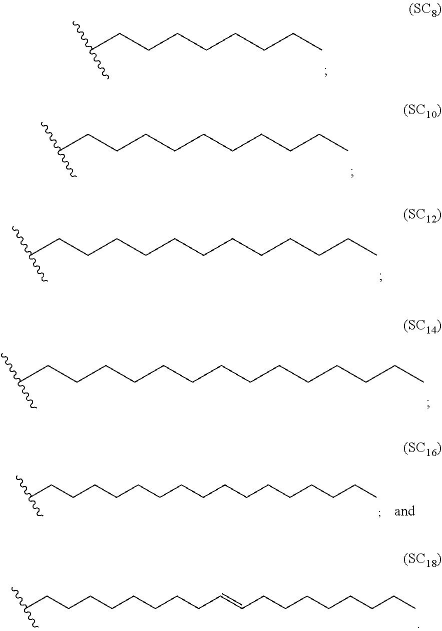

In certain aspects, R is:

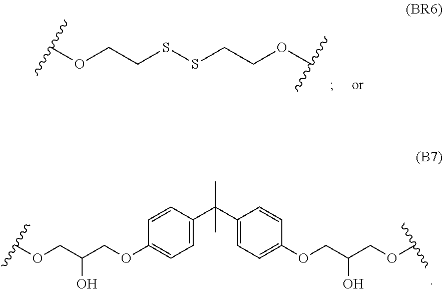 [SMILES: *OCCSSCCO* ; *OCC(O)COc1ccc(C(C)(C)c2ccc(OCC(O)CO*)cc2)cc1]

In certain aspects, R′ is:

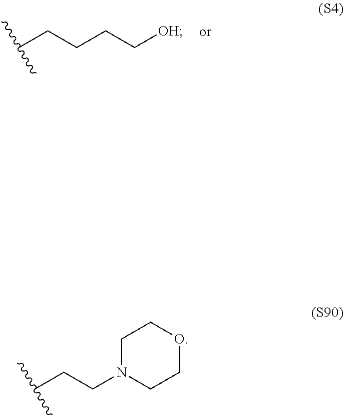 [SMILES: *CCCCO ; *CCN1CCOCC1]

In certain aspects, R″ is selected from the group consisting of:

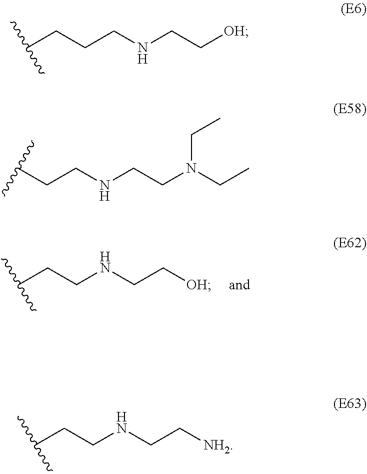 [SMILES: *CCCNCCO ; *CCNCCN(CC)CC ; *CCNCCO ; *CCNCCN]

In certain aspects, —(CH₂)ₘ₁—(C═C)_{q}—(CH₂)ₘ₂—CH₃ is selected from the group consisting of Sc12, Sc16, and Sc18.

In particular aspects, the compound of formula (I) comprises:

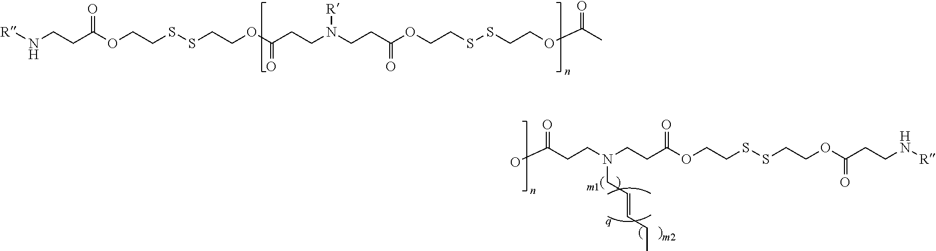

In particular aspects, the compound of formula (I) comprises:

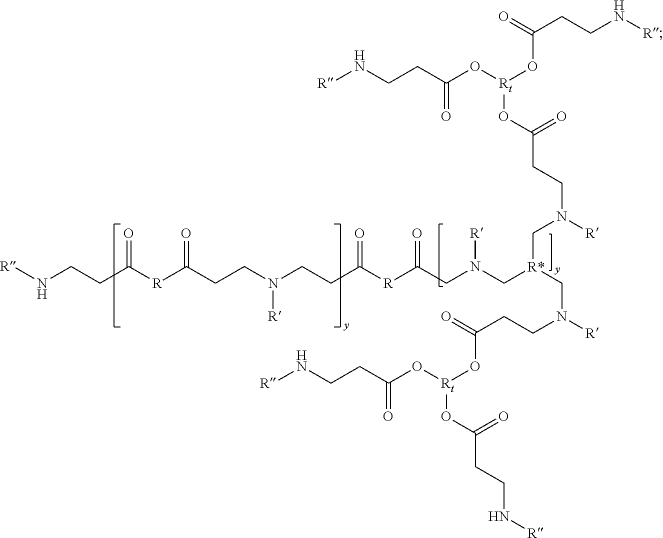

wherein each R* is a triacrylate, quanternary, or hexafunctional acrylate monomer selected from the group consisting of:

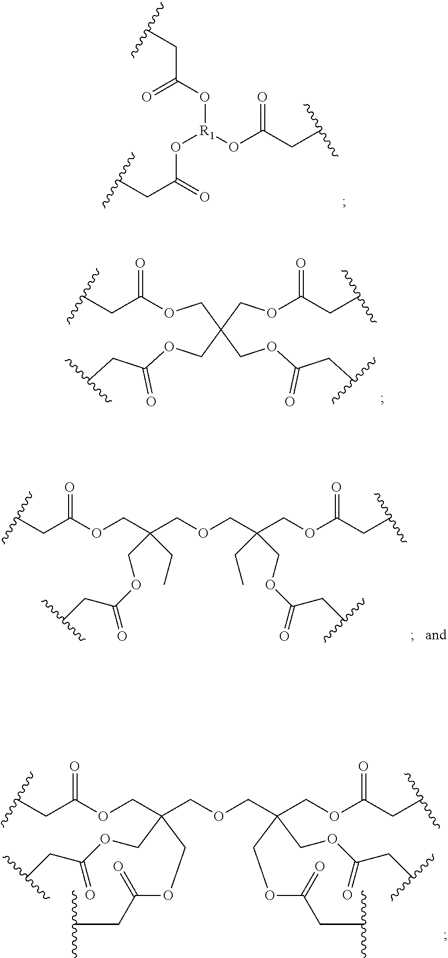

wherein each Rₜ is independently a trivalent group; and each y is independently an integer from 1 to 10,000.

In some aspects, the composition has a ratio of the hydrophobic side chain to the hydrophilic side chain is between about 10:90 hydrophobic side chain:hydrophilic side chain to about 90:10 hydrophobic side chain:hydrophilic side chain.

In particular aspects, the composition is selected from the group consisting of BR6-S4, Sc16-E6, 50%/50% ratio of S4/Sc16; BR6-S4, Scl6-E62, 50%/50% ratio of S4/Sc16; BR6-S4, Sc16-E63, 50%/50% ratio of S4/Sc16; BR6-S4, Sc18-E6, 50%/50% ratio of S4/Sc18; BR6-S4, Sc18-E62, 50%/50% ratio of S4/Sc18; and BR6-S4, Sc18-E63, 50%/50% ratio of S4/Sc18.

In other aspects, the composition is selected from the group consisting of B7-S90, Sc12-E6, 50%/50% ratio of S90/Sc12; B7-S90, Sc12-E6, 20%/80% ratio of S90/Sc12; B7-S90, Sc12-E58, 50%/50% ratio of S90/Sc12; B7-S90, Sc12-E58, 20%/80% ratio of S90/Sc12; B7-S90, Sc12-E63, 50%/50% ratio of S90/Sc12; and B7-S90, Sc12-E63, 20%/80% ratio of S90/Sc12.

In certain aspects, the composition comprises a weight ratio between the polymer and the nucleic acid from between about 30 w/w and about 200 w/w. In particular aspects, the weight ratio between the polymer and the nucleic acid is between about 50 w/w and about 150 w/w.

In certain aspects, the one or more nucleic acids comprise DNA or RNA. In particular aspects, the one or more nucleic acids are selected from the group consisting of an oligonucleotide, a cyclic dinucleotide, plasmid DNA, linear DNA, siRNA, miRNA, mRNA, and combinations thereof. In more particular aspects, the one or more nucleic acids is mRNA. In certain aspects, the mRNA comprises a self-amplifying mRNA (SAM).

In other aspects, the composition further comprises one or more immunomodulatory nucleic acids. In certain aspects, the one or more immunomodulatory nucleic acids are selected from the group consisting of CpG, GpG, poly(I:C), and a cyclic dinucleotide (CDN).

In some aspects, the composition comprises a nanoparticle comprising a compound of formula (I) and one or more nucleic acids. In certain aspects, the composition comprises a nanoparticle encapsulating an mRNA that encodes an autoreactive antigen. In particular aspects, the autoreactive antigen comprises myelin oligodendrocyte glycoprotein (MOG). In certain aspects, the composition further comprises GpG. In certain aspects, the composition further comprises rapamycin.

In some aspects, the composition further comprises lipid-PEG. In certain aspects, the lipid-PEG is admixed with the compound of formula (I) at a varying mass percent. In particular aspects, the lipid-PEG is selected from the group consisting of 1,2-dimyristoyl-rac-glycero-3-methoxypolyethylene glycol 2000 (DMG-PEG2k) and C18-PEG2k. In certain aspects, the composition comprises DMG-PEG2k at a weight percent from about 2 wt % to about 10 wt %. In particular aspects, the composition further comprises a nanoparticle comprising a compound of formula (I), lipid-PEG, and one or more nucleic acids. In more particular aspects, the nanoparticle has a zeta-potential that varies with a varying mass percent of lipid-PEG.

In some aspects, the composition further comprises one or more excipients. In certain aspects, the one or more excipients include one or more cryoprotectants, one or more sugar alcohols, MgCl₂, and combinations thereof. In more certain aspects, the one or more cryoprotectants comprise a sugar. In particular aspects, the sugar is selected from the group consisting of glucose, fructose, sorbitol, mannitol, sucrose, trehalose, and raffinose. In certain aspects, the one or more sugar alcohols comprise sorbitol.

In some aspects, the composition is lyophilized. In certain aspects, the composition comprises a storable powder.

In some aspects, the presently disclosed subject matter provides a genetic vaccine comprising the presently disclosed compositions, wherein the genetic vaccine targets one or more antigen presenting cells. In some aspects, the one or more nucleic acids comprises an mRNA encoding one or more antigens and/or one or more immunomodulatory nucleic acids selected from the group consisting of CpG, GpG, poly(I:C), and cyclic dinucleotides (CDN).

In some aspects, the presently disclosed subject matter provides a method for delivering one or more nucleic acids to a subject, the method comprising administering a presently disclosed composition or presently disclosed genetic vaccine to the subject.

In certain aspects, the administering comprises systemically administering the composition or the genetic vaccine to the subject. In particular aspects, the method comprises intravenously administering the composition to the subject.

In some aspects, the presently disclosed subject matter provides a method for treating a disease or condition, the method comprising administering a presently disclosed composition or the presently disclosed genetic vaccine to a subject in need of treatment thereof.

In certain aspects, the disease or condition is selected from the group consisting of a cancer, an infectious disease, and an autoimmune disease.

In particular aspects, the infectious disease is selected from the group consisting of a coronavirus, influenza, and rabies.

In particular aspects, the cancer is selected from the group consisting of a solid tumor and a metastatic cancer.

In certain aspects, the cancer comprises a solid tumor in one or more organs selected from the group consisting of the brain, colon, breast, prostate, liver, kidney, lung, esophagus, head and neck, ovaries, cervix, stomach, colon, rectum, bladder, uterus, testes, and pancreas.

In certain aspects, the cancer comprises a metastatic cancer selected from the following types of cancer and metastasis sites: bladder: bone, liver, lung; breast: bone, brain, liver, lung; colon: liver, lung, peritoneum; kidney: adrenal gland, bone, brain, liver, lung; lung: adrenal gland, bone, brain, liver, other lung; melanoma: bone, brain, liver, lung, skin, muscle; ovary: liver, lung, peritoneum; pancreas: liver, lung, peritoneum; prostate: adrenal gland, bone, liver, lung; rectal: liver, lung, peritoneum; stomach: liver, lung, peritoneum; thyroid: bone, liver, lung; and uterus: bone, liver, lung, peritoneum, vagina.

In certain aspects, the autoimmune disease is selected from the group consisting of type I diabetes mellitus (T1D), Crohn's disease, ulcerative colitis, myasthenia gravis, vitiligo, Graves' disease, Hashimoto's disease, Addison's disease and autoimmune gastritis, autoimmune hepatitis, primary biliary cirrhosis, autoimmune thrombocytopenia, rheumatoid arthritis, systemic lupus erythematosus, progressive systemic sclerosis and variants, polymyositis and dermatomyositis, inflammatory bowel disease, celiac disease, inflammatory myositis, Sjogren's syndrome, multiple sclerosis, psoriasis and scleroderma.

In particular aspects, the subject is a human or an animal. In yet more particular aspects, the method is selected from a prophylactic treatment method, a therapeutic treatment method, and combinations thereof.

In some aspects, the presently disclosed subject matter provides a kit, the kit comprising one or more of: one or more compounds of formula (I), one or more nucleic acids, reagents, and instructions for use.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows representative monomers and a general reaction scheme for the synthesis of branched and lipophilic PBAE-based bioreducible polymers made and tested by high-throughput and high-content evaluation;

FIG. 2 shows representative monomers and a general reaction scheme for the synthesis of branched and lipophilic PBAE-based non-bioreducible polymers;

FIG. 3A and FIG. 3B demonstrate that unlike older PBAE structures (referred to herein as “1st-gen PBAEs”), the presently disclosed lipophilic PBAE structures (referred to herein as “next-generation lipophilic PBAEs”) can transfect immortalized and primary dendritic cells in vitro. (FIG. 3A) Next-generation lipophilic PBAE materials are highly efficient at transfecting immortalized dendritic cells (DCs) and show promise in primary DCs compared to 1st-gen PBAEs. (FIG. 3B) Novel PBAE structures #1 (BR6-S4, Sc12-based) and #2 (BR6-S4, Sc18-based) with c12 and c18 lipid sidechains, respectively (Older PBAE from left to right: BR6-S4-E6, BR6-S4-E62; Novel PBAE #1 from left to right: BR6-S4, Sc12-E6, BR6-S4, Sc12-E62 (50% Sc12 for both); Novel PBAE #2 from left to right: BR6-S4, Sc18-E6, BR6-S4, Sc18-E63, BR6-S4, Sc18-E62, BR6-S4, Sc18-E61 (50% Sc18 for all), are superior to 1st-gen PBAEs in transfecting primary DCs;

FIG. 4A and FIG. 4B demonstrate that nanoparticles were formed from linear, lipophilic PBAEs self-assembled with mRNA. The mRNA NPs were dialyzed to replace all solvents with a neutral, isotonic vehicle (1′PBS) and were injected into Balb/c mice (10 μg mRNA/100 μL volume per mouse). (FIG. 4A) Increasing amounts of lipophilic side chain correlates with higher mRNA transfection after intravenous (IV) injection. (FIG. 4B) Small changes to polymer end-cap structure result in significant differences in transfection efficiency;

FIG. 5A, FIG. 5B, FIG. 5C, FIG. 5D, and FIG. 5E show: (FIG. 5A) Linear lipophilic PBAE (B7-S90, Sc12-E63, 80% Sc12) and mRNA were mixed to form NPs and injected intravenously, after which they transfect the spleen, liver, and lymph nodes. (FIG. 5B) The Ai9 reporter mouse allows sensitive and quantitative measurement of in vivo transfection by polymeric mRNA nanoparticles. (FIG. 5C) mRNA NPs (B7-S90, Sc12-E63, 80% Sc12, 30 w/w ratio of polymer to mRNA) transfect 5-10% of splenic DCs after IV injection of mRNA NPs. (FIG. 5D) Transfection efficiency is greatest in DCs among cell types measured and (FIG. 5E) >70% of transfected cells stain positive for DC markers;

FIG. 6A, FIG. 6B, and FIG. 6C show: (FIG. 6A) The Ai9 reporter mouse allows sensitive and quantitative measurement of in vivo transfection by LPBAE/mRNA NPs; (FIG. 6B) Transfection and liver targeting, represented as fluorescence, can be optimized by varying the LPBAE chemical structure; and (FIG. 6C) Top NP formulations can transfect up to 1-10% of APCs in the liver and spleen;

FIG. 7A and FIG. 7B demonstrate that linear, bioreducible, lipophilic PBAE BR6-S4, Sc18-E62 was used to form NPs with mRNA and GpG oligonucleotide and injected into Ai9 mice. Transfected cells were identified and characterized by flow cytometry. (FIG. 7A) mRNA NPs on their own do not cause innate activation of APCs in Ai9 mice (left), and co-encapsulation of a tolerogenic agent, GpG, does not cause global immune suppression. Pre-treatment of mice with systemic TLR4 agonist CpG does increase CD86 expression overall in DCs (left) and in transfected DCs (right), but incorporation of GpG into the NPs reduces the elevated expression of CD86. (FIG. 7B) Splenic DCs are significantly (p<0.05, one-way ANOVA, Dunnet post-test) more activated after systemic CpG administration, which is alleviated by delivery of GpG along with mRNA in NPs;

FIG. 8 demonstrates that linear, bioreducible, lipophilic PBAE BR6-S4, Sc18-E62 was used to form NPs with mRNA and poly(I:C) or CpG adjuvant and injected into Ai9 mice. Transfected cells were identified and characterized by flow cytometry. mRNA NPs on their own do not cause innate activation of APCs, but this property can be tailored by co-delivering nucleic acid adjuvants within the same NPs;

FIG. 9A and FIG. 9B demonstrate that PBAE was used to form NPs with DNA encoding myelin oligodendrocyte glycoprotein (MOG) antigen and IL-10; (FIG. 9A) in an experimental autoimmune encephalomyelitic (EAE) mouse model of multiple sclerosis, mice were injected IV (red arrows) with control DNA or DNA encoding MOG and an immunosuppressive cytokine, resulting in suppression of symptoms during treatment. (FIG. 9B) Mice were injected IV with the 1st-generation DNA NPs used in (A) or with 2nd-generation mRNA NPs, which showed significantly higher transfection, especially to liver (see also FIG. 28);

FIG. 10 demonstrated that linear lipophilic PBAE 7-90, c12-E63 was used to form NPs with mRNA, then dialyzed against 1′PBS with or without a lipid-PEG moiety (DMG-PEG) for surface coating. Surface coating with PEG-lipids improves expression after IV injection. Statistical significance calculated using Student's t-test with Welch's correction;

FIG. 11A, FIG. 11B, and FIG. 11C show: (FIG. 11A) Incorporation of DMG-PEG2000 into LPBAE NPs significantly improved mRNA expression when dialyzed into nanoparticle mix (Student's t-test with Welch's correction); (FIG. 111B) Chemical 20 structures of PEG-lipids that can be used in to improve NP stability and transfection efficacy; and (FIG. 11C) Ionizable lipids can be incorporated into NP formulations to improve liver targeting;

FIG. 12 demonstrates that linear lipophilic PBAEs surface-coated with DMG-PEG show good NP stability when complexed with RNA and incubated at 37° C. in physiological fluids for up to 3-5 days;

FIG. 13A, FIG. 13B, FIG. 13C, and FIG. 13D demonstrate that (FIG. 13A) Excipients, such as sucrose, can protect NPs during the lyophilization process and preserve their transfection capability, but the best formulation may vary among types of NPs. (FIG. 13B) Common small-molecule excipients can drastically improve lyophilization stability. (FIG. 13C) Leading lyophilized NP formulations do not lose significant transfection efficacy if stored at −20° C. for at least 2 years. (FIG. 13D) L-PBAE/mRNA NPs successfully transfect cells in vivo after freeze/thaw or lyophilization;

FIG. 14A, FIG. 14B, and FIG. 14C demonstrate bioreducible lipophilic PBAE-mRNA nanoparticles (NPs) as cancer vaccine. (FIG. 14A) Schematic of the mRNA-based cancer vaccine technology using polymeric (PBAE) NPs. (FIG. 14B) Reaction scheme for bioreducible lipophilic PBAEs. The bioreducible diacrylate backbone monomer (R) is polymerized with a 1:1 mixture of a hydrophilic amine sidechain monomer (S4) and a lipophilic amine side chain monomer (Sc12-18) via Michael addition. The obtained diacrylate-terminated random copolymer is endcapped with an amine-containing monomer (A-E) to form the final polymer structure. (FIG. 14C) Monomers used in the combinatorial library synthesis to form bioreducible lipophilic PBAEs;

FIG. 15A, FIG. 15B, FIG. 15C, FIG. 15D, FIG. 15E, FIG. 15F, and FIG. 15G show the characterization of bioreducible lipophilic PBAE polymers and mRNA nanoparticles (NPs). (FIG. 15A) Molecular weights of PBAEs of varying lipophilicity assessed by GPC. (FIG. 15B) Hydrodynamic diameter of PBAE NPs formed at a 300 or 200 w/w ratio of polymer to mRNA assessed via DLS (n=2). Significance indicates comparison to non-lipophilic PBAE nanoparticles (ROD) at the respective w/w ratio. (FIG. 15C) Representative TEM images of R18D mRNA NPs (scale bar=200 nm). (FIG. 15D) Surface charge of mRNA PBAE NPs in PBS (n=2). (FIG. 15E) Encapsulation efficiency of mRNA assessed by the Ribogreen assay (n=3). Significance indicates comparison to non-lipophilic PBAE nanoparticles (ROD) at the respective w/w ratio. (FIG. 15F) Encapsulation and dissociation of fluorescently labeled mRNA and CpG ODN from R18D-based NPs formed at 300 and 100 w/w ratios after incubation in PBS over 4 hours assessed by a gel electrophoresis assay. (FIG. 15G) mRNA dissociation assessed in 10% serum for non-lipophilic (ROD) and lipophilic (R18D) NPs formed at 300 and 100 w/w ratios over 4 h. Error bars represent SEM. *P<0.05, **P<0.01, and ***P<0.001;

FIG. 16A, FIG. 16B, FIG. 16C, FIG. 16D, FIG. 16E, and FIG. 16F demonstrate transfection of dendritic cells (DCs) in vitro by bioreducible lipophilic PBAE mRNA nanoparticles (NPs). (FIG. 16A) Polymer library was evaluated for 30 transfection of the murine dendritic cell line DC2.4 using mRNA encoding GFP. Cells were treated with NPs formed at 200 w/w and a dose of 50 ng mRNA/well and transfection efficiency was assessed via flow cytometry after 24 h. (FIG. 16B) Representative brightfield (BF) and fluorescent microscopy images of DC2.4 cells transfected with non-lipophilic R0A or lipophilic R18A GFP mRNA NPs (scale bar=50 nm). (FIG. 16C) Transfection of DC2.4 cells by top-performing R18D NPs was assessed at various mRNA doses and compared to leading commercial mRNA transfection reagent Lipofectamine MessengerMAX. (FIG. 16D) A subset of the polymer library was evaluated on murine BMDCs using luciferase-encoding mRNA. Cells were treated with NPs at a dose of 25 ng mRNA/well and bioluminescence activity was assessed after 24 h to determine transfection levels normalized to cell viability. (FIG. 16E) Polymers with Sc18 monomer were synthesized with 50:50 or 75:25 ratio of lipophilic side chain monomer Sc18 to hydrophilic side chain monomer S4. DC2.4 cells were treated with GFP mRNA NPs with varied lipophilicity at a dose of 25 ng mRNA/well, and transfection was assessed after 24 h. (FIG. 16F) The transfection efficiency in DC2.4s was examined following treatment with R18D NPs co-encapsulating GFP mRNA and CpG or poly(I:C) adjuvants with varied mRNA to adjuvant ratios where the mRNA dose was kept constant (25 ng/well) after 24 h. Significance indicates comparison to no adjuvant control. Error bars represent SEM (n=4). ***P<0.001 and ****P<0.0001;

FIG. 17A, FIG. 17B, FIG. 17C, FIG. 17D, and FIG. 17E demonstrate cellular uptake and endosomal escape following mRNA nanoparticle (NP) design. (FIG. 17A) DC2.4 cells and (FIG. 17B) murine primary BMDCs were treated with NPs carrying Cy5-mRNA at a dose of 50 ng mRNA/well and NP uptake was assessed at 6 h post-treatment by flow cytometry (n=4). (FIG. 17C) DC2.4 cells were treated with R18D NPs co-encapsulating mRNA at 50 ng mRNA/well and FITC-CpG, and uptake of CpG was assessed 6 h post-treatment by flow cytometry (n=4). (FIG. 17D) Representative images of DC2.4 cells labeled with lysosome/endosome dye 6 h post-treatment with NPs carrying Cy5-labeled GFP-encoding mRNA to visualize cellular uptake, NP colocalization with endosomes/lysosomes, and GFP transfection (scale bars=20 m). (FIG. 17E) Manders' coefficient was determined using ImageJ to quantify the degree of colocalization between NPs and endosomes/lysosomes (n=3). Error bars represent SEM. *P<0.05, **P<0.01, and ****P<0.0001;

FIG. 18A, FIG. 18B, FIG. 18C, FIG. 18D, FIG. 18E, FIG. 18F, FIG. 18G, FIG. 18H, and FIG. 181 demonstrate in vivo transfection in spleen following systemic administration of R18D mRNA nanoparticles (NPs). (FIG. 18A) R18D NPs carrying luciferase mRNA (mLuc) (10 μg/mouse) and CpG (2.5 μg/mouse) or poly(I:C) (0.1 μg/mouse) were assembled at a polymer-to-nucleic acid ratio of 100 w/w and administered intravenously to C57BL/6J mice. Whole animal bioluminescence imaging was performed 6 h after administration. (FIG. 18B) Image analysis was used to assess total flux in spleen. (FIG. 18C) Schematic of Ai9 mouse model used to assess transfected cell types in vivo following systemic administration of mRNA NPs carrying Cre mRNA. Cells that are transfected undergo Cre recombinase-mediated recombination, resulting in tdTomato expression that is detected by flow cytometry. (FIG. 18D-FIG. 18H) R18D Cre mRNA NPs were administered intravenously to Ai9 mice at 10 μg mRNA/mouse and tdTomato expression in key cell populations in the spleen was assessed after 24 h. (FIG. 18D) Percent of all tdTomato+(tdT+) cells in spleen that are DCs, macrophages, or monocytes. (FIG. 18E) Pie charts indicating 10 average share of transfected cells in the spleen belonging to each cell population shown for NP treatments carrying no adjuvant, 2.5 μg CpG, or 0.1 μg poly(I:C). (FIG. 18F) Percent of DCs in the spleen that are transfected. (FIG. 18G) Representative flow cytometry plots showing transfected tdTomato+DCs treated with mRNA-NP formulations co-encapsulating no adjuvant, 2.5 μg CpG, or 0.1 μg poly(I:C). (FIG. 18H) Geometric mean fluorescent intensity (MFI) of CD40 and CD86 expression in all splenic DCs. (FIG. 181) Representative histograms of CD40 and CD86 expression in no treatment control, and following NP treatment co-encapsulating no adjuvant, 2.5 μg CpG, or 0.1 μg poly(I:C). Error bars represent SEM;

FIG. 19A, FIG. 19B, FIG. 19C, FIG. 19D, FIG. 19E, FIG. 19F, and FIG. 19G demonstrate in vivo therapeutic efficacy of PBAE mRNA nanoparticle (NP) vaccination in B16-OVA and B16-F10 mouse melanoma models. (FIG. 19A-FIG. 19E) 3×10⁵ B16-OVA cells were inoculated subcutaneously in the right flank of C57BL/6J mice on day 0, and R18D NPs encapsulating Luciferase-encoding mRNA (as an irrelevant mRNA control) or OVA-encoding mRNA were administered intravenously (IV) on days 4 and 9 at 10 μg mRNA/mouse and 2.5 μg CpG or 0.1 μg poly(I:C) for adjuvant groups (n=7-8 mice/group). 200 μg of aPD-1 was injected intraperitoneally (I.P.) on day 5. (FIG. 19A) Tumor growth measurements showing the in vivo therapeutic effects between the treatment groups. *P<0.05, **P<0.01 and ****P<0.0001 for comparison between aPD-1+mOVA/CpG NP treatment 30 group and respective controls (indicated by color). *P<0.05, ##P<0.01 and ####P<0.0001 for comparison between aPD-1+mOVA/p(I:C) NP treatment group and respective controls (indicated by color). (FIG. 19B) Mice were euthanized once tumors reached 200 mm², and survival curves are shown. (FIG. 19C) Mice were bled on day 14 post-inoculation, and the percent of OVA-specific CD8+ T cells out of total CD8+ T cells in the blood was assessed using H2Kb SIINFEKL tetramer staining. Significance indicates comparison of mOVA/CpG and mOVA/p(I:C) NP treatment compared to all respective controls. (FIG. 19D) On day 32 post-inoculation, surviving mice in mOVA/CpG and mOVA/p(I:C) NP treatment groups were bled and presence 5 of OVA-specific CD8+ T cells was assessed via tetramer staining. (FIG. 19E) Representative flow cytometry plots showing BV421 H2Kb SIINFEKL tetramer staining in CD3+CD8+cells in all groups. (FIG. 19F-FIG. 19G) 3×10⁵ B16-F10 cells were inoculated subcutaneously in the right flank of C57BL/6J mice on day 0, and R18D NPs encapsulating luciferase mRNA or a 1:1 mixture of TRP2 and GP100-encoding mRNA (10 μg total mRNA/mouse) and CpG (2.5 μg/mouse) were administered I.V. on days 4 and 9 (n=7-8 mice/group). 200 μg of aPD-1 was injected I.P. on day 5. (FIG. 19F) Tumor growth measurements showing the in vivo therapeutic effects between the treatment groups. **P<0.01, ***P<0.001 and ****P<0.0001 for comparison between aPD-1+mTRP2/mGP100/CpG NP treatment group and aPD-1 control (black) or aPD-1+mLuc/CpG NP group (pink). *P<0.05 for comparison between aPD-1+mLuc/CpG NP group and aPD-1 group. (FIG. 19G) Mice were euthanized once tumors reached 200 mm², and survival curves are shown. Error bars represent SEM;

FIG. 20A, FIG. 20B, FIG. 20C, FIG. 20D, FIG. 20E demonstrate in vivo therapeutic efficacy of PBAE mRNA nanoparticle (NP) vaccination in MC38-OVA mouse colon carcinoma model. (FIG. 20A) 1×10⁶ MC38-OVA cells were inoculated subcutaneously in the right flank of C57BL/6J mice on day 0, and R18D NPs encapsulating luciferase-encoding mRNA or OVA-encoding mRNA (10 μg mRNA/mouse) and CpG (2.5 μg/mouse) were administered intravenously on days 9 and 14 (n=7-8 mice/group). 200 μg of aPD-1 was injected intraperitoneally on day 10. Tumor growth measurements showing the in vivo therapeutic effects between the treatment groups. Significance indicates comparison of aPD-1+mOVA/CpG NP treatment group to aPD-1 group (black) or aPD-1+mLuc/CpG NP group (pink). (FIG. 20B) Mice were euthanized once tumors reached 200 mm², and survival curves are shown. (FIG. 20C) 4 mice were randomly selected from each group to be bled on day 21 post-inoculation, and the percent of OVA-specific CD8+ T cells out of total CD8+ T cells in the blood was assessed using H2Kb SIINFEKL tetramer staining. (FIG. 20D) Percent of CD8+ T cells out of total CD3+ T cells in blood is shown. (FIG. 20E) Representative flow cytometry plots showing BV421 H2Kb SIINFEKL tetramer staining in CD3+CD8+cells in all groups. Error bars represent SEM;

FIG. 21A, FIG. 21B, and FIG. 21C demonstrate cell viability of dendritic cells (DCs) following in vitro transfection by bioreducible lipophilic PBAE mRNA nanoparticles (NPs). (FIG. 21A) A polymer library was screened for toxicity on the murine dendritic cell line DC2.4. Cells were treated with NPs formed at 200 w/w and a dose of 50 ng mRNA/well and metabolic activity was assessed after 24 h via the MTS assay and normalized to untreated cells. Significance indicates comparison to untreated control. (FIG. 21B) A subset of the polymer library was screened on murine bone-marrow derived dendritic cells (BMDC). Cells were treated with NPs at a dose of 25 ng mRNA/well and metabolic activity was assessed after 24 h and normalized to untreated cells. (FIG. 21C) Polymers with Sc18 monomer were synthesized with 50:50 or 75:25 ratio of lipophilic side chain monomer Sc18 to hydrophilic side chain monomer S4. Viability of DC2.4s following treatment of mRNA NPs with varied lipophilicity was assessed after 24 h via MTS assay and normalized to untreated cells. Error bars represent SEM (n=4). *P<0.05, ***P<0.001, and ****P<0.0001;

FIG. 22 demonstrates transfection of BMDCs with R18D nanoparticles (NPs) compared to leading commercial mRNA transfection reagents. Murine BMDCs were transfected with R18D GFP-mRNA NPs at 250 ng mRNA/well (n=4 replicates) or 20 with jetMessenger or Lipofectamine MessengerMAX (leading mRNA transfection reagents for hard-to-transfect cells) at 500 ng mRNA/well (n=3) in a 24-well plate. Error bars represent SEM. ****P<0.0001;

FIG. 23A and FIG. 23B show cellular uptake of mRNA nanoparticles (NPs) 24 h post-treatment. (FIG. 23A) DC2.4 cells and (FIG. 23B) murine BMDCs were treated with NPs carrying Cy5-mRNA at a dose of 50 ng mRNA/well, and NP uptake was assessed 24 h post-treatment by flow cytometry (n=4) (C) DC2.4 cells were treated with R18D NPs co-encapsulating mRNA at a dose of 50 ng mRNA/well, and FITC-labeled CpG and uptake of CpG was assessed 24 h post-treatment by flow cytometry (n=4). Error bars represent SEM. **P<0.01 and ****P<0.0001;

FIG. 24A, FIG. 24B, and FIG. 24C demonstrate in vivo transfection over time following systemic administration of R18D mRNA nanoparticles (NPs). R18D NPs carrying luciferase mRNA (10 μg/mouse) were administered intravenously in C57BL/6 mice. (FIG. 24A) Whole animal bioluminescence imaging was performed 2, 6, 24, 48, and 96 hr post-administration. (FIG. 24B) Mice were euthanized at each timepoint and major organs (liver, spleen, kidney, heart, and lungs) and inguinal lymph nodes (LN) were dissected out and imaged by IVIS. Representative organ images at 2, 6, and 24 hr timepoints are shown. (FIG. 24C) Total flux in spleen until endpoint of 96 hr post-treatment. Error bars represent SEM;

FIG. 25A, FIG. 25B, FIG. 25C, FIG. 25D, and FIG. 25E demonstrate in vivo transfection in splenic cell populations following systemic administration of R18D mRNA nanoparticles (NPs). R18D Cre mRNA NPs were administered intravenously to Ai9 mice at 10 μg mRNA/mouse with CpG and poly(I:C) adjuvants at varying adjuvant doses and w/w ratios of polymer to total nucleic acid. tdTomato expression in key cell populations in the spleen was assessed 24 h post-treatment via flow cytometry. (FIG. 25A) Percent tdTomato+cells in all splenocytes. (FIG. 25B) CD40 expression in tdTomato+transfected splenic DCs. (FIG. 25C) CD86 expression in tdTomato+transfected splenic DCs. (FIG. 25D) Percent tdTomato transfection in various cell types in the spleen. (FIG. 25E) Percent of all transfected tdTomato+splenocytes that belong to each cell type. Error bars represent SEM;

FIG. 26 shows individual tumor growth curves for B16-OVA mRNA nanoparticle (NP) vaccination study. 3×10⁵ B16-OVA cells were inoculated subcutaneously in the right flank of C57BL/6 mice on day 0, and R18D NPs encapsulating Luciferase-encoding mRNA (as an irrelevant mRNA control) or OVA-encoding mRNA were administered intravenously on days 4 and 9 at 10 μg mRNA/mouse and 2.5 μg CpG or 0.1 μg poly(I:C) for adjuvant groups (n=7-8 mice/group). 200 μg of aPD-1 was injected intraperitoneally on day 5. Tumor area was measured every other day beginning on day 7 post-inoculation, and individual tumor growth curves for each treatment group are shown;

FIG. 27 shows individual tumor growth curves for B16-F10 mRNA nanoparticle (NP) vaccination study. 3×10⁵ B16-F10 cells were inoculated subcutaneously in the right flank of C57BL/6 mice on day 0, and R18D NPs encapsulating luciferase-encoding mRNA or a 1:1 mixture of TRP2- and GP100-encoding mRNA (10 μg total mRNA/mouse) and CpG (2.5 μg/mouse) at were administered intravenously on days 4 and 9 (n=7-8 mice/group). 200 μg of aPD-1 was injected intraperitoneally on day 5. Tumor area was measured every other day beginning on day 7 post-inoculation, and individual tumor growth curves for each treatment group are shown; and

FIG. 28 demonstrates tolerogenic nanoparticle vaccines for treating multiple sclerosis. Left panel: EAE progression (prophylactic). Right panel: EAE progression (therapeutic). Mice treated prophylactically with nanoparticles containing antigen (MOG) mRNA with or without GpG and rapamycin exhibited reduced disease progression compared to controls. Mice treated therapeutically after disease onset with nanoparticles containing GpG and rapamycin also exhibited reduced disease progression compared to controls. The nanoparticles were injected in the inguinal lymph nodes.

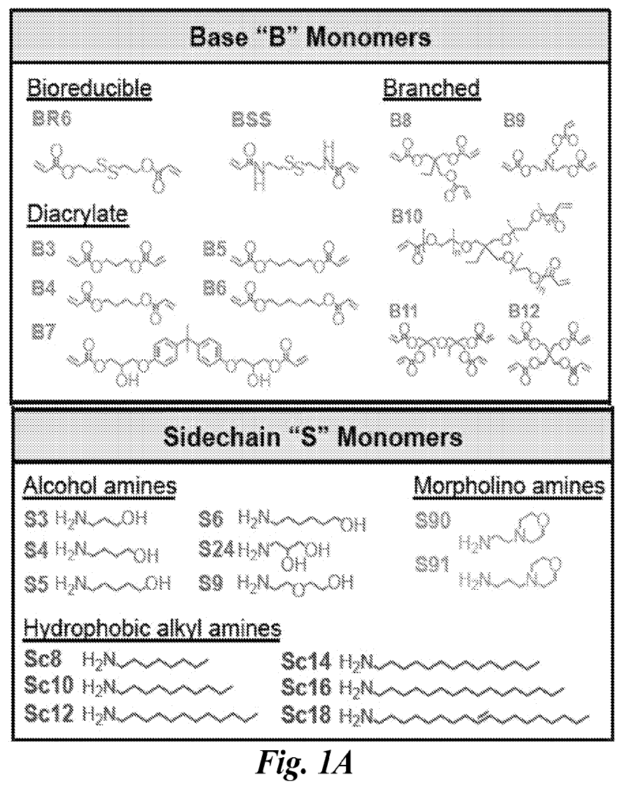

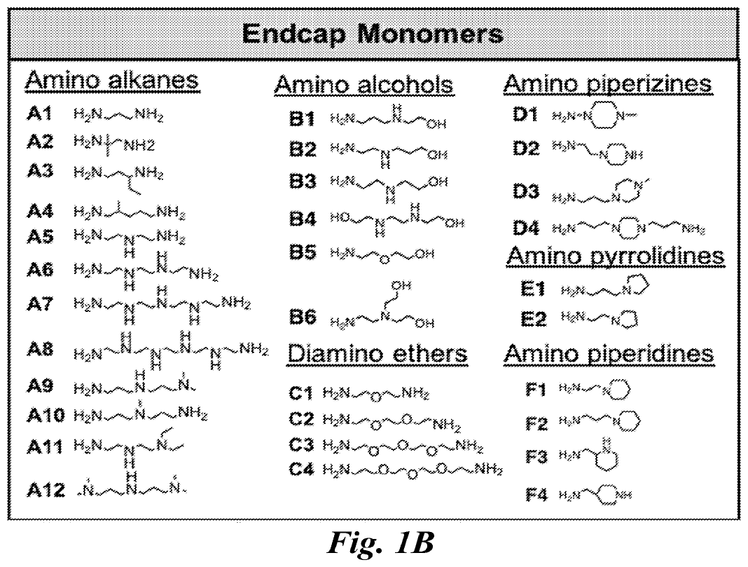

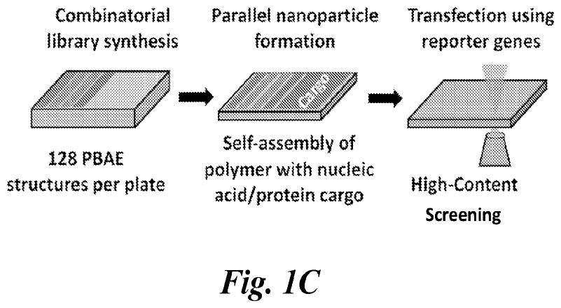

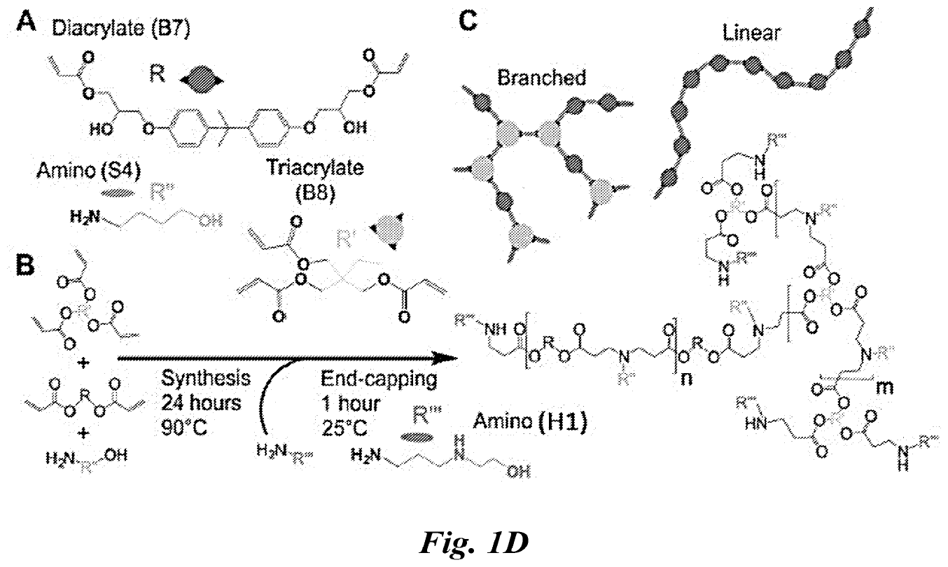

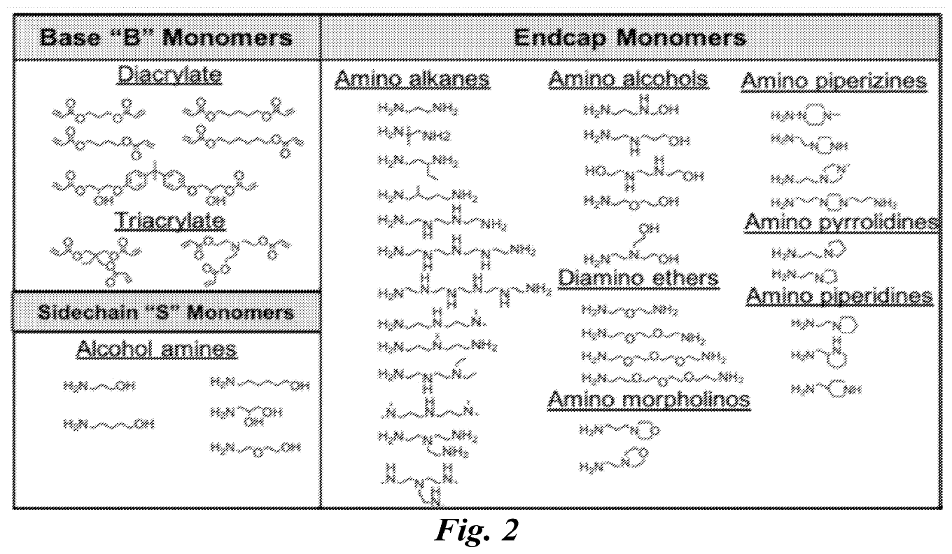

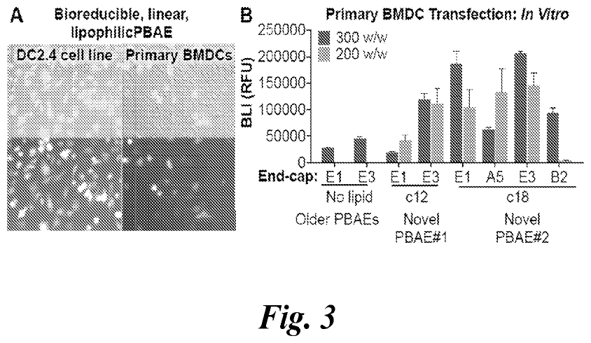

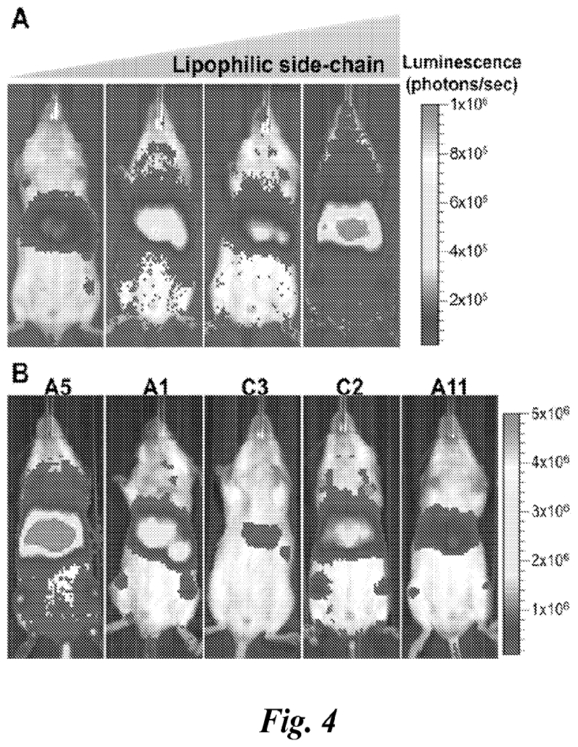

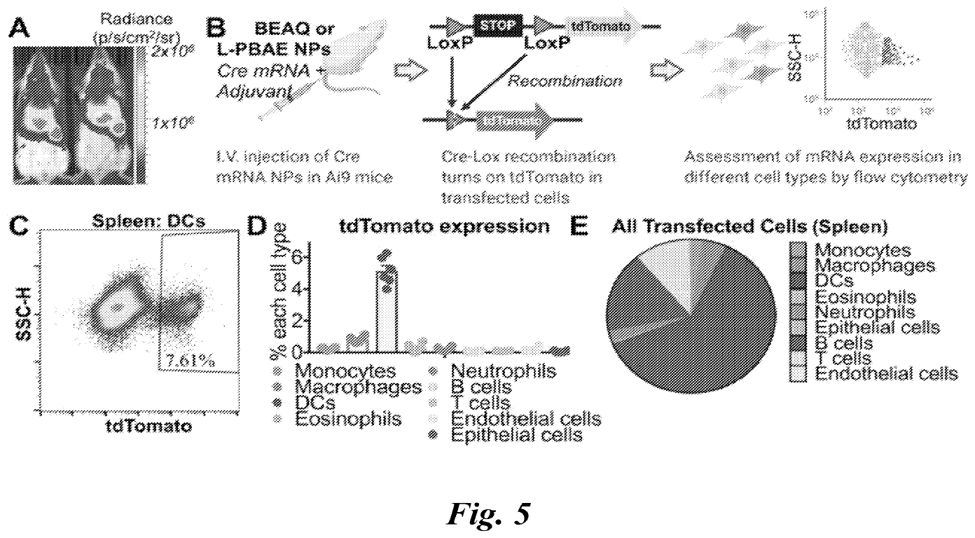

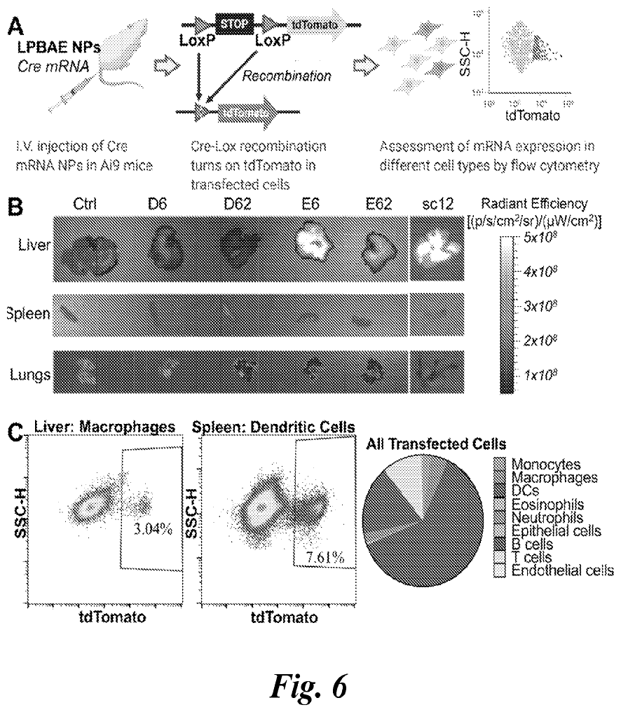

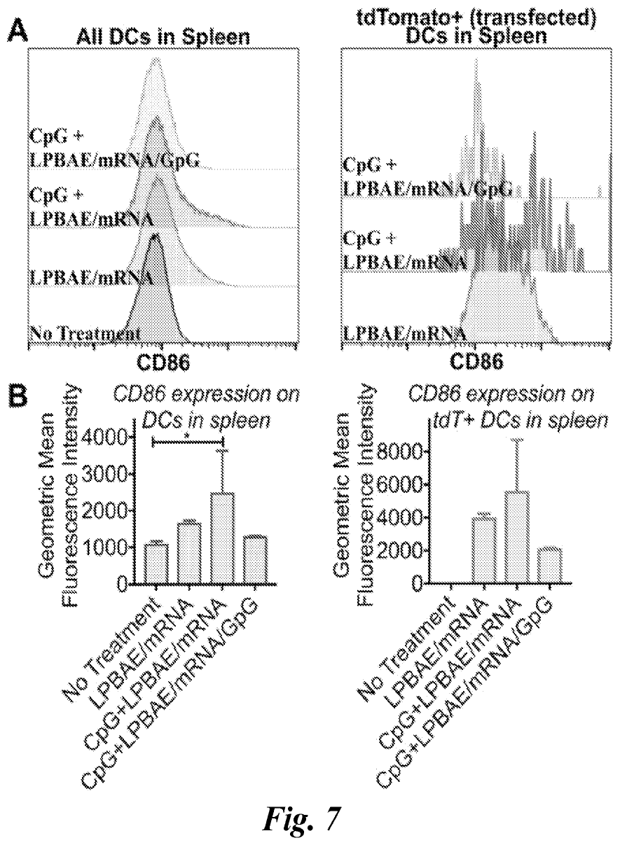

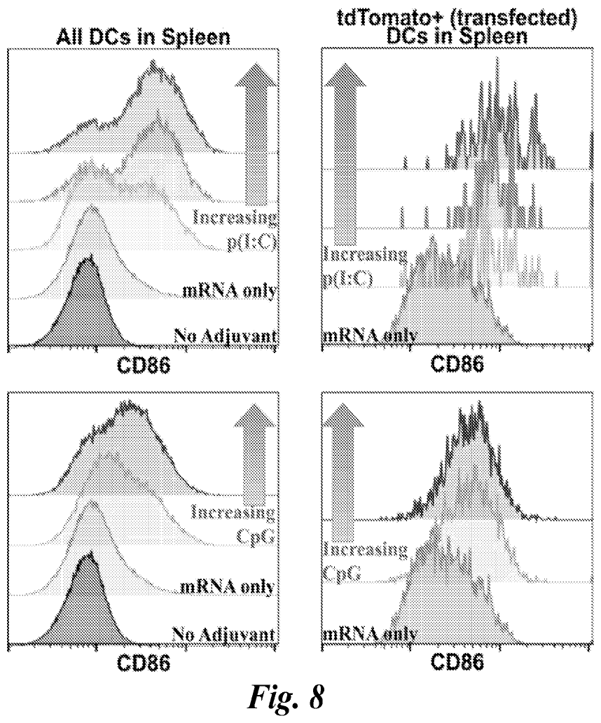

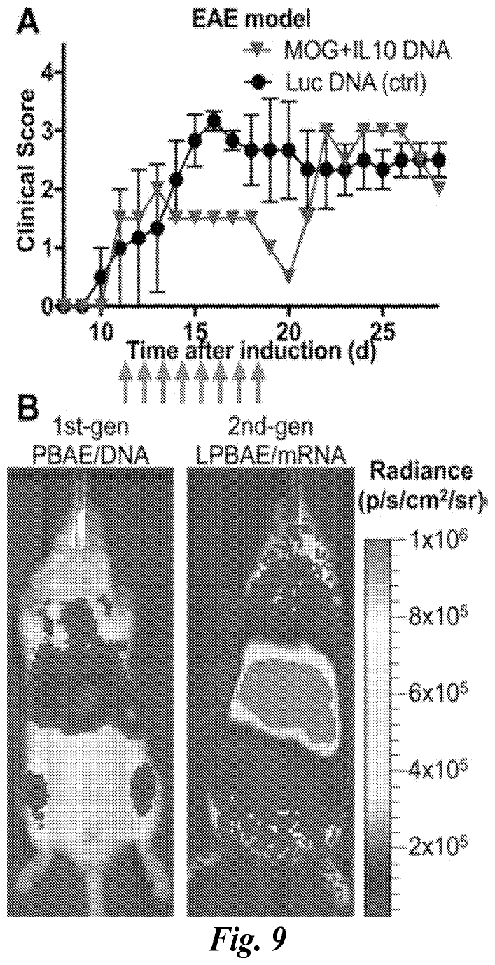

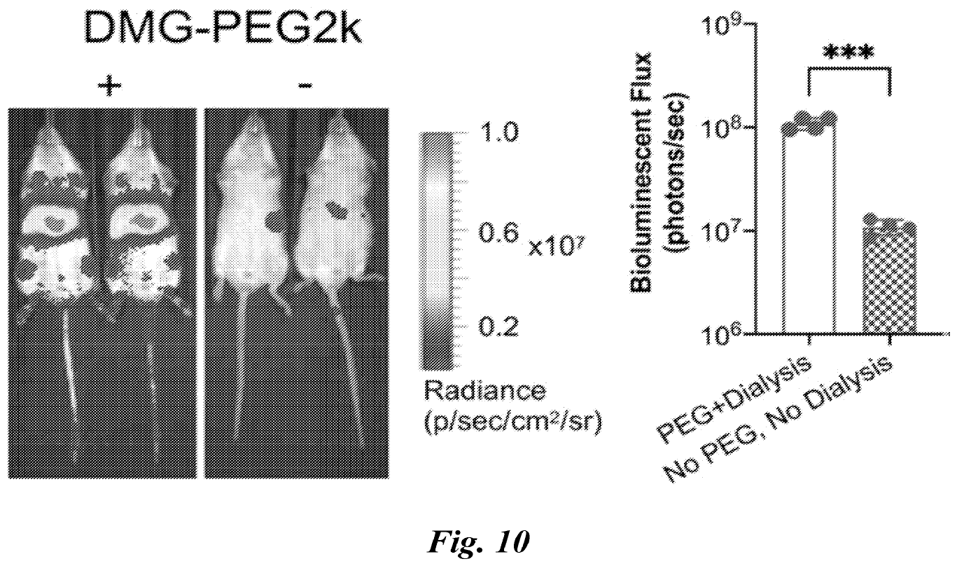

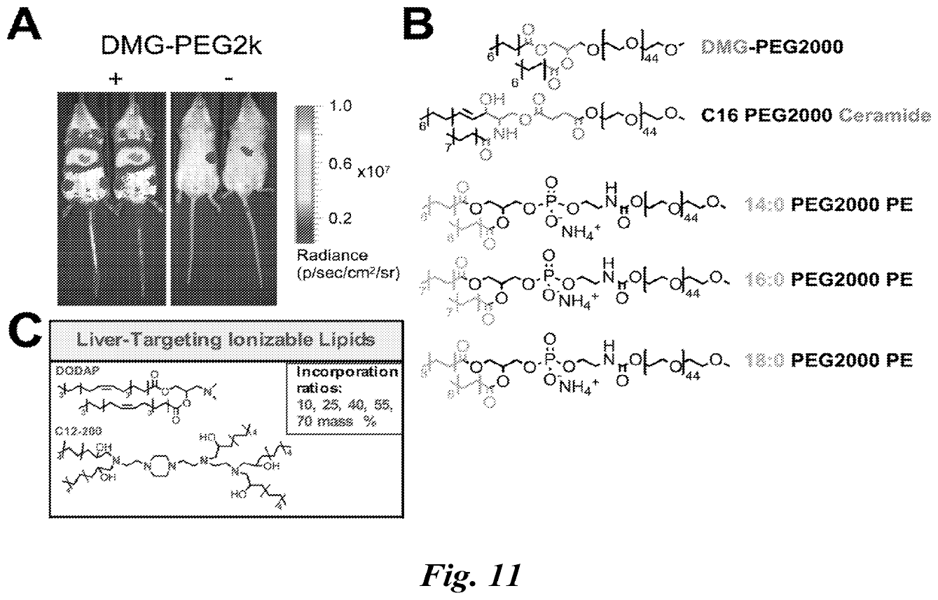

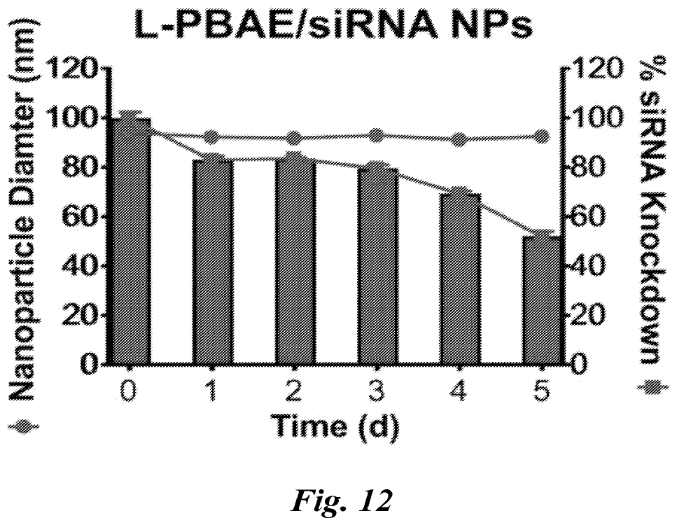

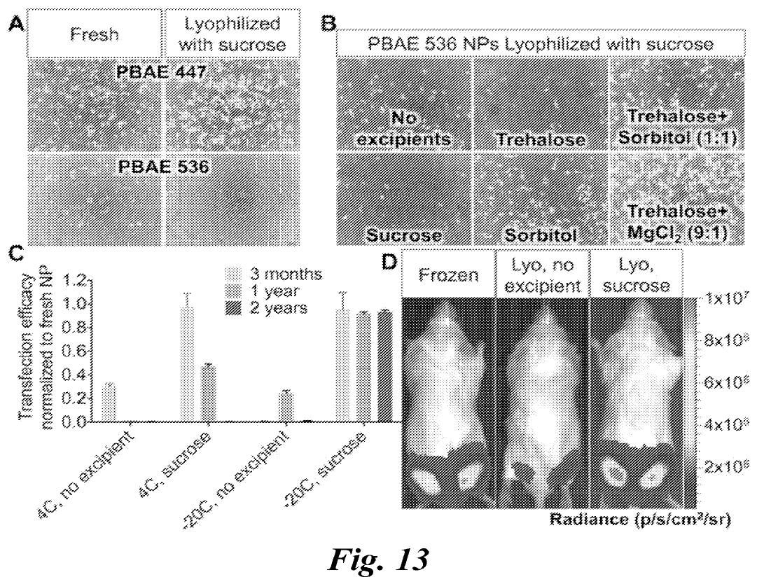

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Polymeric Nanoparticle Genetic Vaccines

The presently disclosed subject matter provides compositions comprising degradable polymers combined with nucleic acids, such as DNA and RNA, encoding antigen and their use as genetic vaccines.

The degradable polymer structures include: (1) ester linkages for biodegradation. In particular embodiments, the degradable polymer structure includes disulfide linkages in the polymer backbone. The polymer backbone further includes other numbers of carbon atoms; (2) a hydrophobic amino-alkyl chain as a side chain, wherein the side chain includes an alkylene chain comprising about ten or more carbon atoms, including between 10 and 20 carbon atoms, including 10, 11, 12, 13, 14, 15, 16, 17, 18, 19, and 20 carbon atoms, and wherein the alkylene chain can be saturated or unsaturated); (3) a hydrophilic amino side chain that contains at least one oxygen; and (4) an end capping molecule containing an amine group.

The presently disclosed materials provide a new generation of genetic vaccines, including mRNA vaccines, that can be injected systemically to target antigen-presenting cells. This characteristic can evoke stronger cellular immune responses against infectious diseases including, but not limited to COVID-19, cancers, including many types of tumors, and/or can be used to treat autoimmune diseases, including, but not limited to, multiple sclerosis.

### A. Compositions

In some embodiments, the presently disclosed subject matter provides a composition comprising a polymer of formula (I) and one or more nucleic acids encoding antigen:

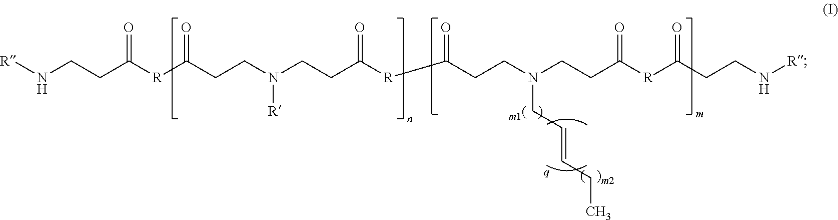

wherein m and n are each independently an integer from 1 to 10,000; m1 is an integer selected from 0, 1, 2, 3, 4, 5, 6, 7, 8, 9, and 10; m2 is an integer selected from the group consisting of 1, 2, 3, 4, 5, 6, 7, 8, 9, 10, 11, 12, 13, 14, 15, 16, 17, 18, 19, and 20; and q is an integer selected from 0 or 1;
wherein —(CH₂)ₘ₁—(C═C)_{q}—(CH₂)ₘ₂—CH₃ comprises a hydrophobic sidechain;
R comprises a divalent radical comprising a biodegradable ester linkage and/or a bioreducible disulfide linkage;
R′ is hydrophilic sidechain comprising a monovalent radical derived from a hydrophilic amine monomer;
R″ is monovalent radical derived from an amine-containing end capping group; and pharmaceutically acceptable salts thereof.

As used herein, “biodegradable” polymers and/or nanoparticles are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). Such components preferably do not induce inflammation or other adverse effects in vivo. In some instances, the chemical reactions relied upon to break down the biodegradable compounds are uncatalyzed.

In certain embodiments, the biodegradable polymers and/or nanoparticles comprise a chemical moiety having one or more degradable linkages, such as an ester linkage, a disulfide linkage, an amide linkage, an anhydride linkage, and a linkage susceptible to enzymatic degradation. Representative degradable linkages include, but are not limited to:

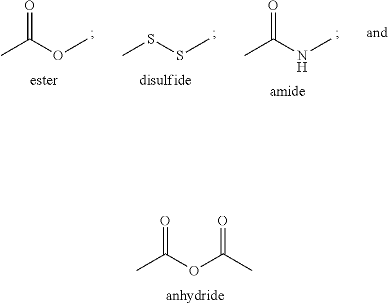

In particular, disulfide bonds can be degraded reductively, i.e., are bioreducible, in the human body via glutathione (GSH), which is present predominantly in the cytosol of human tissues at concentrations ranging from about 1 mM/to about 8 mM, which is three orders of magnitude greater than the concentration in blood serum (about 5 μM to about 50 μM).

In some embodiments, the biodegradable polymer and/or nanoparticle comprises a poly(beta-amino ester) (PBAE). Exemplary PBAEs suitable for use with the presently disclosed subject matter include those disclosed in:
U.S. Pat. No. 9,884,118 for Multicomponent Degradable Cationic Polymers, to Green et al., issued Feb. 6, 2018;
U.S. Pat. No. 9,802,984 for Biomimetic Peptide and Biodegradable Delivery Platform for the Treatment of Angiogenesis- and Lymphangiogenesis-Dependent Diseases, to Popel et al., issued Oct. 31, 2017;
U.S. Pat. No. 9,717,694 for Peptide/Particle Delivery Systems, to Green et al., issued Aug. 1, 2017;
U.S. Pat. No. 8,992,991 for Multicomponent Degradable Cationic Polymers, to Green et al., issued Mar. 31, 2015;
U.S. Patent Application Publication No. 20180256745 for Biomimetic Artificial Cells: Anisotropic Supported Lipid Bilayers on Biodegradable Micro and Nanoparticles for Spatially Dynamic Surface Biomolecule Presentation, to Meyer et al., published Sep. 13, 2018;
U.S. Patent Application Publication No. 20180112038 for Poly(Beta-Amino Ester)-Co-Polyethylene Glycol (PEG-PBAE-PEG) Polymers for Gene and Drug Delivery, to Green et al., published Apr. 26, 2018;
U.S. Patent Application Publication No. 20170216363 for Nanoparticle Modification of Human Adipose-Derived Mesenchymal Stem Cells for Treating Brain Cancer and other Neurological Diseases, to Quinones-Hinojosa and Green, published Aug. 3, 2017;
U.S. Patent Application Publication No. 20150273071 for Bioreducible Poly (Beta-Amino Ester)s For siRNA Delivery, to Green et al., published Oct. 1, 2015;
U.S. Pat. No. 8,287,849 for Biodegradable Poly(beta-amino esters) and Uses Thereof, to Langer, et al., issued Oct. 16, 2012;
International PCT Patent Application Publication No. WO2020198145 for Gene Delivery Particles to Induce Tumor-Derived Antigen Presenting Cells, to Green, published Oct. 1, 2020; each of which is incorporated by reference in their entirety.

Generally, the presently disclosed multicomponent degradable cationic polymers include a backbone derived from a diacrylate monomer (designated herein below as “B”), an amino-alcohol hydrophilic side-chain monomer (designated herein below as “S”), a hydrophobic side-chain monomer, and an amine-containing end capping monomer (designated herein below as “E”). The end capping group structures are distinct and separate from the polymer backbone structures and the side chain structures of the intermediate precursor molecule for a given polymeric material.

The presently disclosed PBAE compositions can be designated, for example, as B5-S4-E7 or 547, in which R is B5, R′ is S4, and R″ is E7, and the like, where B is the backbone and S is the side chain, followed by the number of carbons in their hydrocarbon chain, e.g., S4 comprises 4 alkylene groups. End capping monomers, E, are sequentially numbered according to similarities in their amine structures. Further, in some embodiments, the presently disclosed PBAE includes a hydrophobic side-chain, which is designated SC-XX, with XX being the number of carbon atoms in the chain.

In some embodiments, R is selected from the group consisting of:

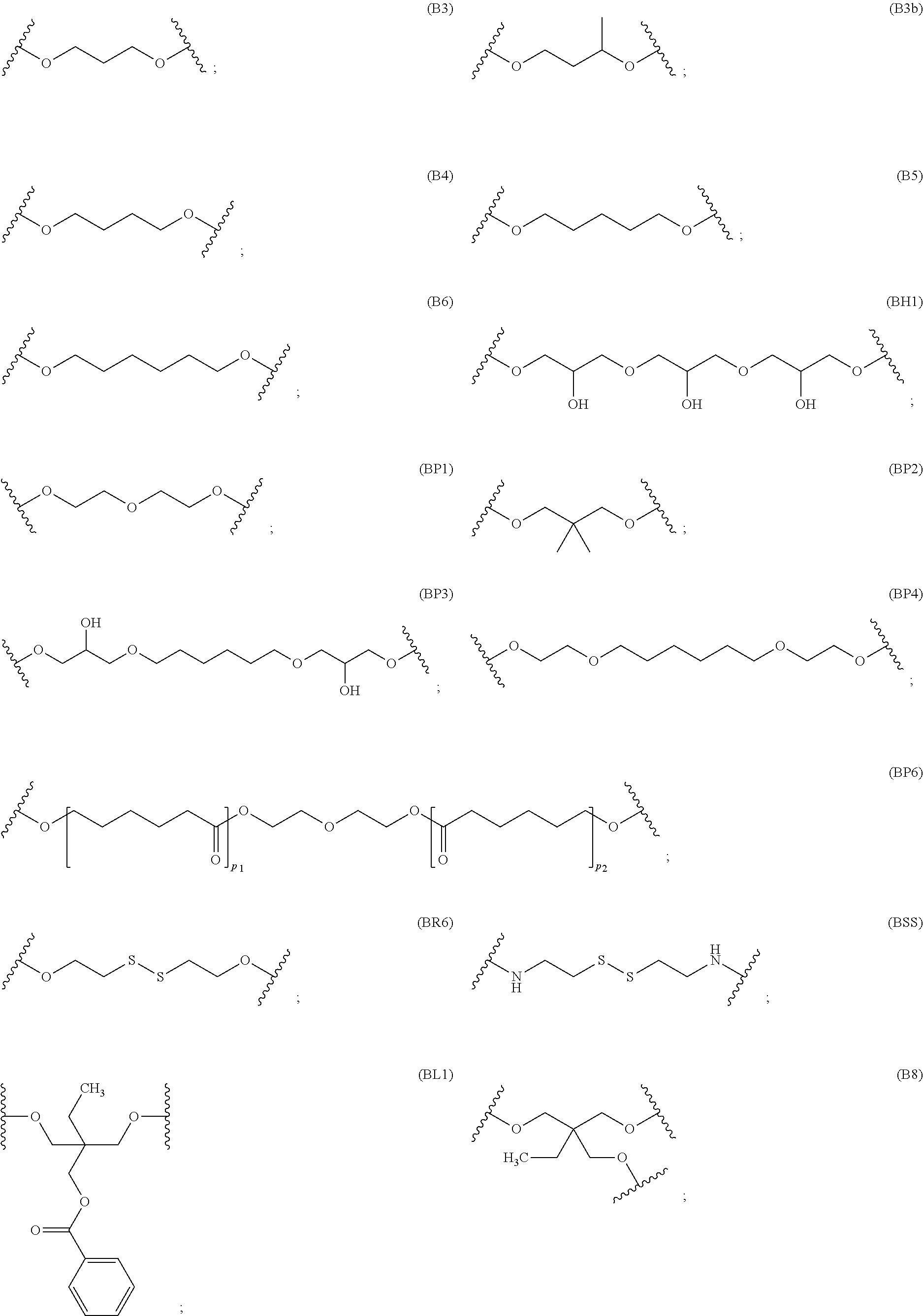

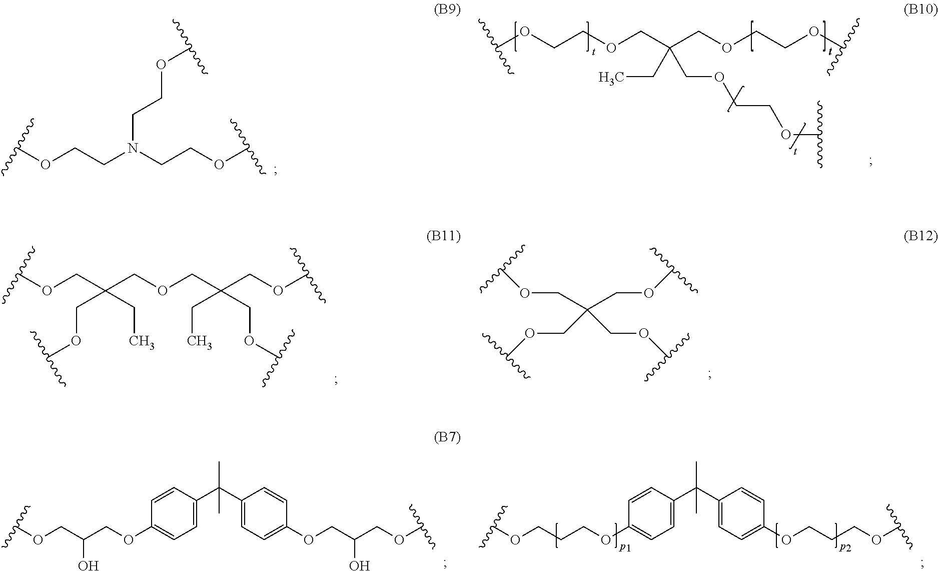

wherein each p1, p2, and t is independently an integer selected from the group consisting of 1, 2, 3, 4, 5, 6, 7, 8, 9, and 10.

In some embodiments, R′ is selected from the group consisting of:

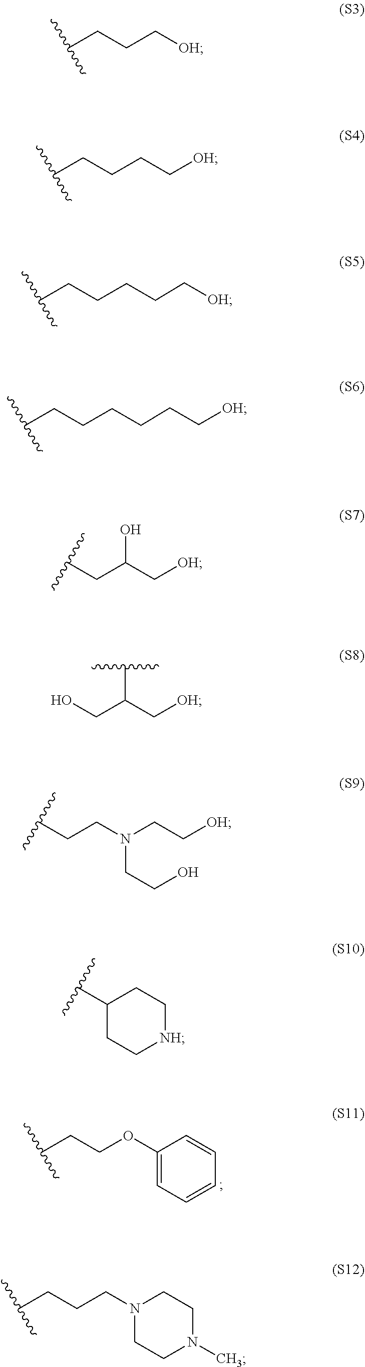

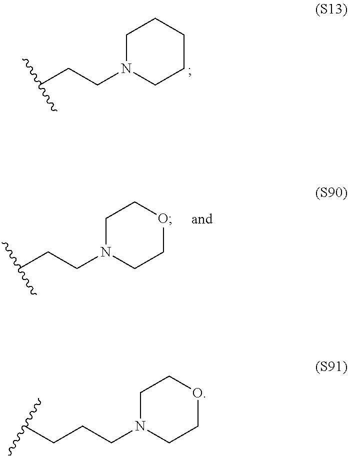 [SMILES: *CCN1CCCCC1 ; *CCN1CCOCC1 ; "and" ; *CCCN1CCOCC1]

In some embodiments, R″ is selected from the group consisting of:

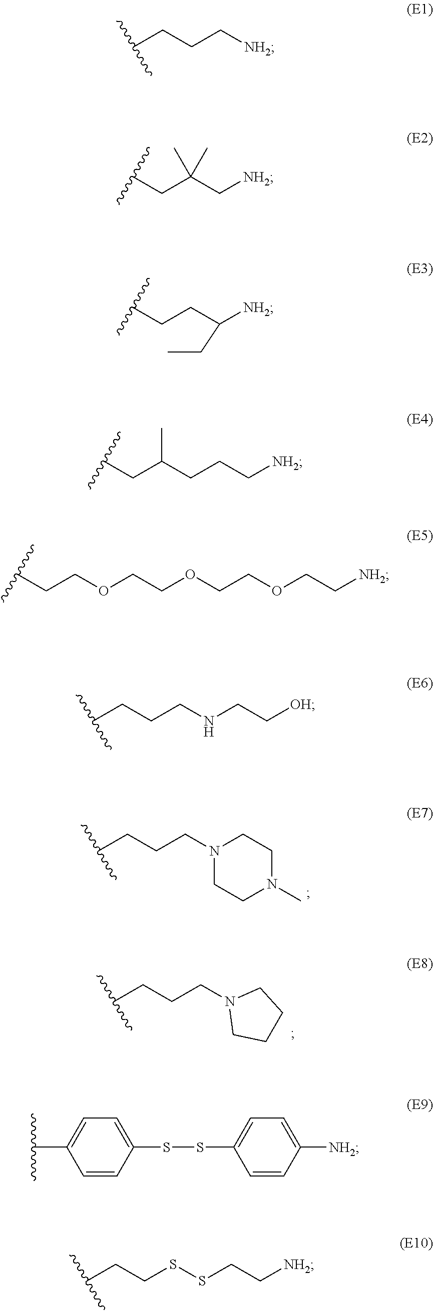

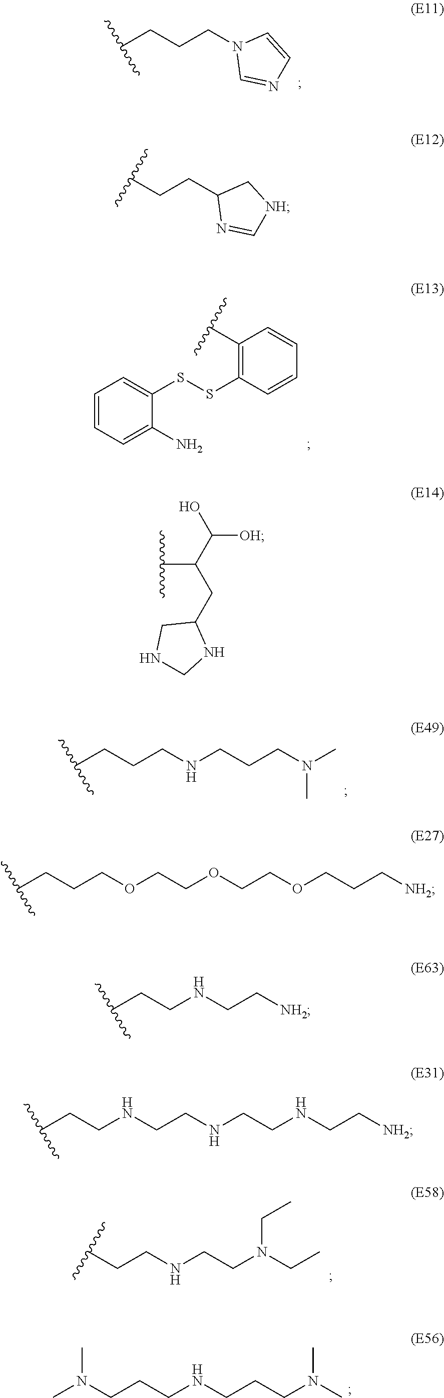

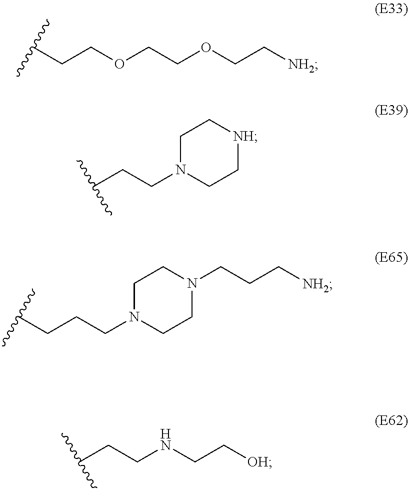 [SMILES: *CCOCCOCCN ; *CCN1CCNCC1 ; *CCCN1CCN(CCCN)CC1 ; *CCNCCO]

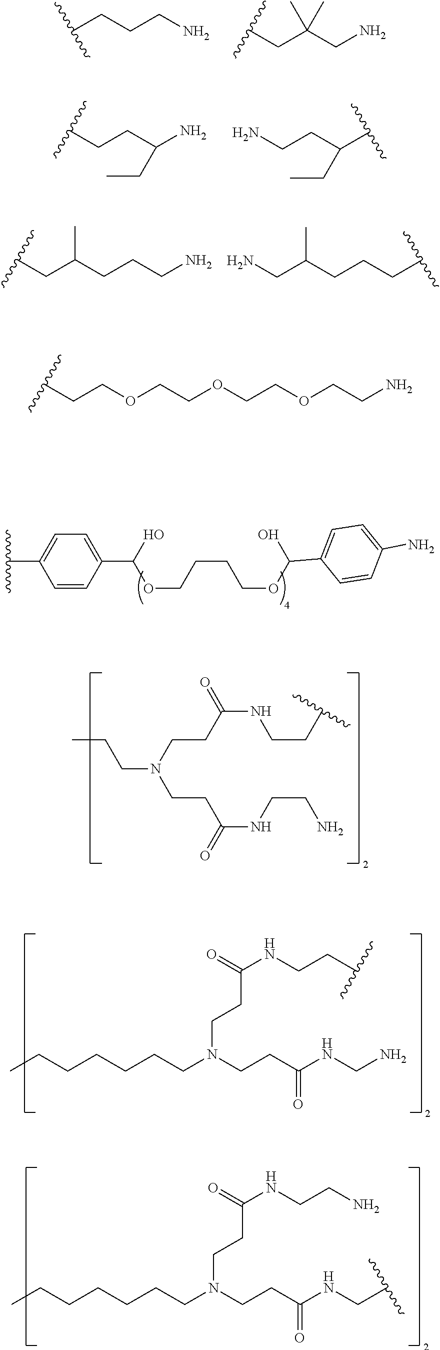

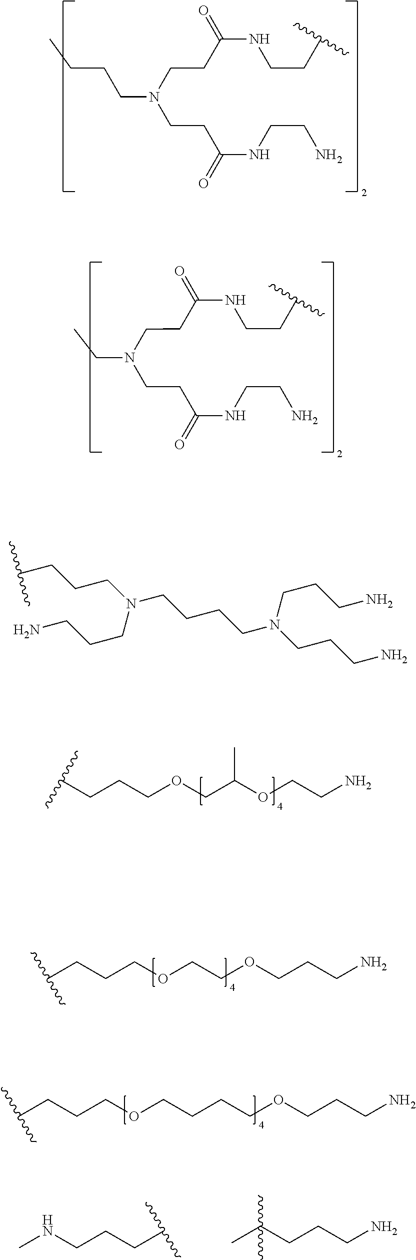

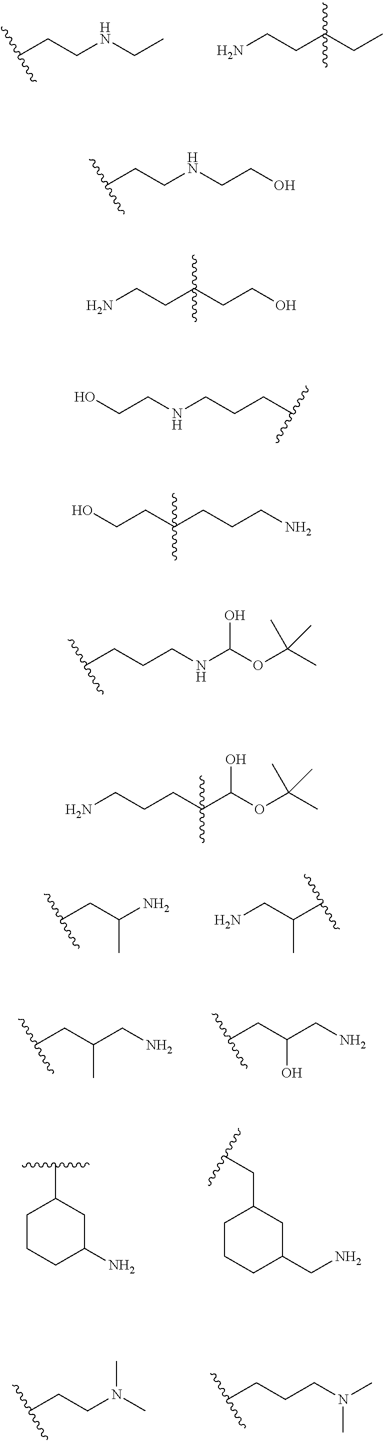

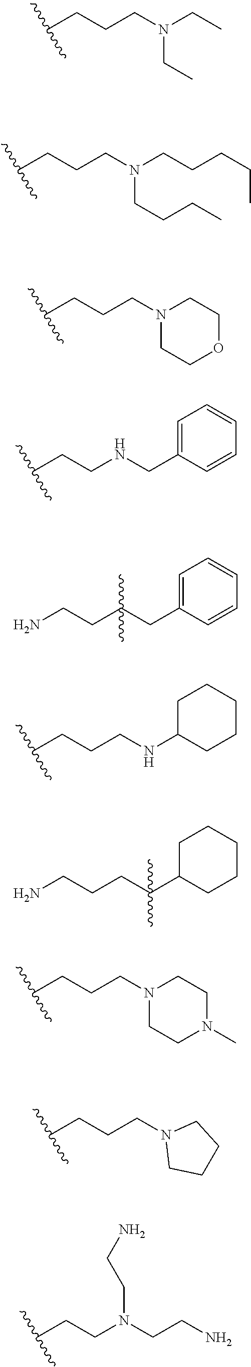

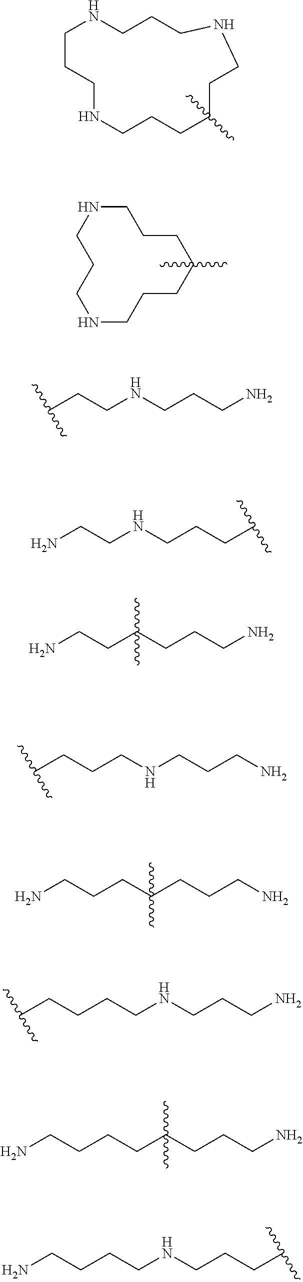

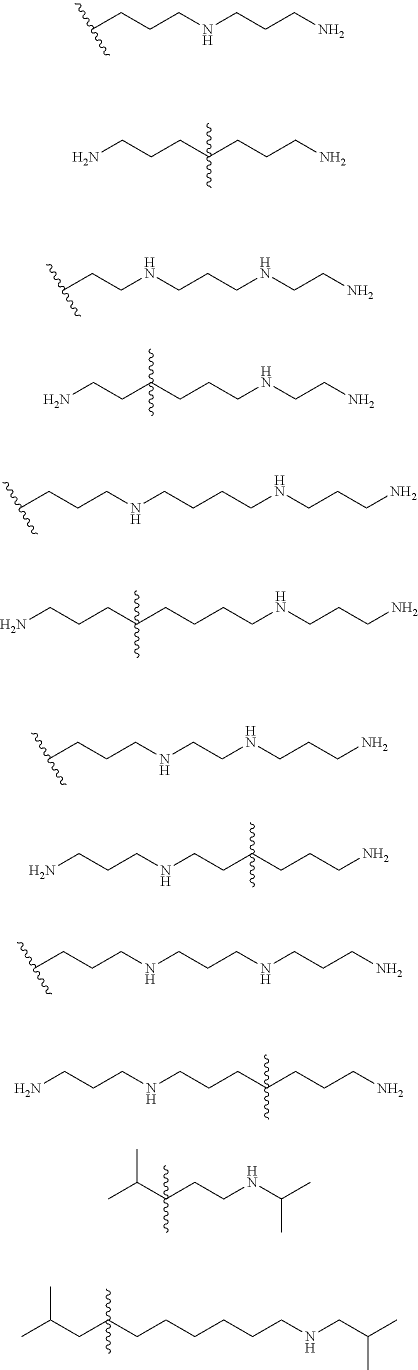

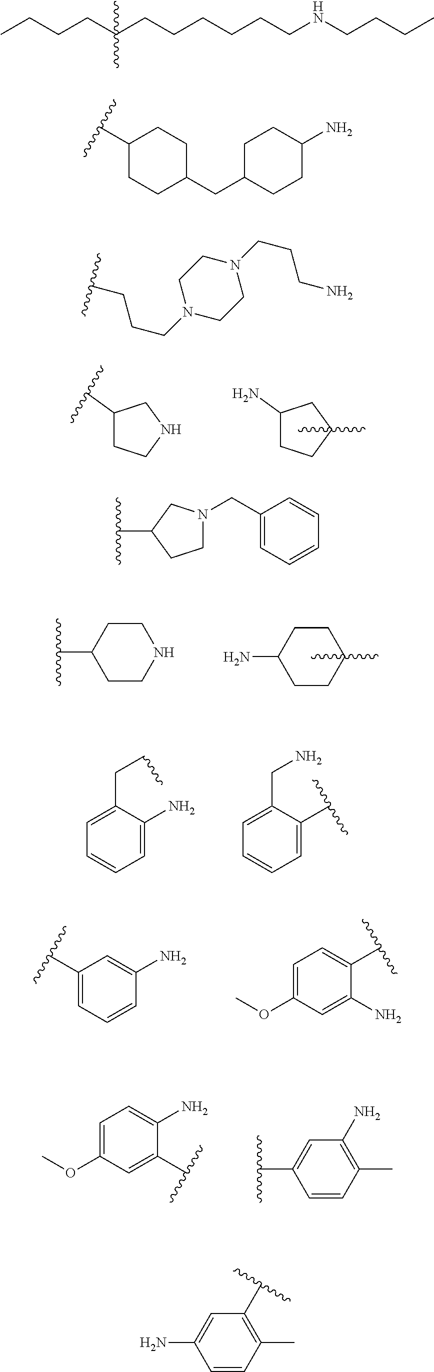

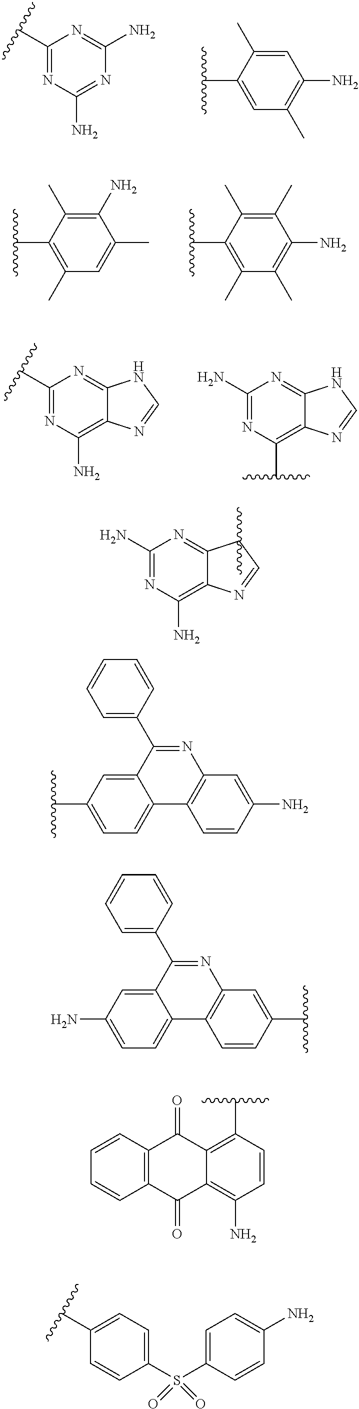

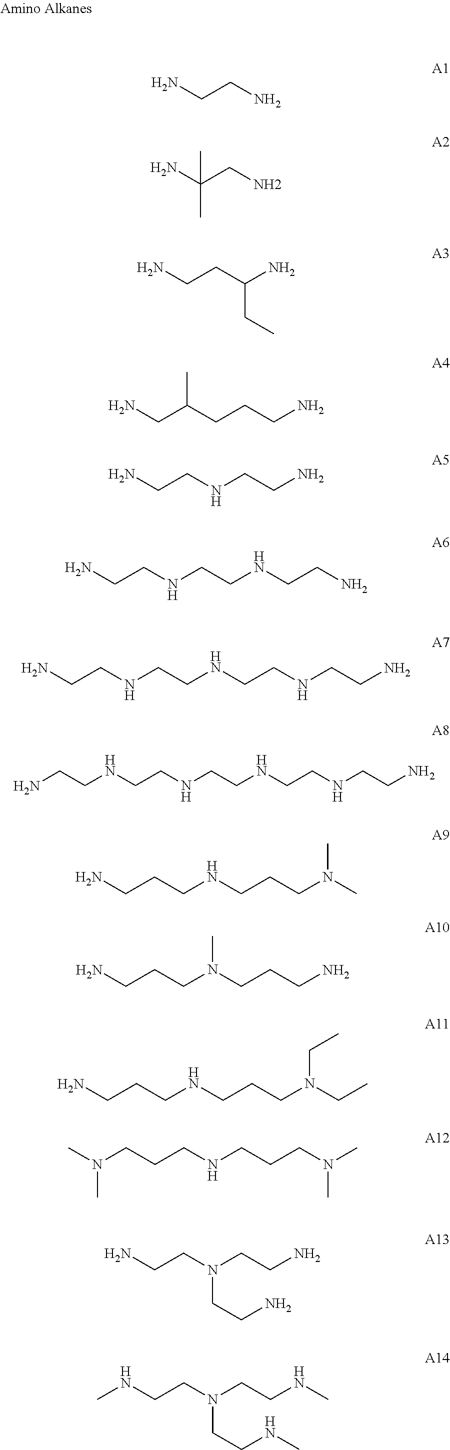

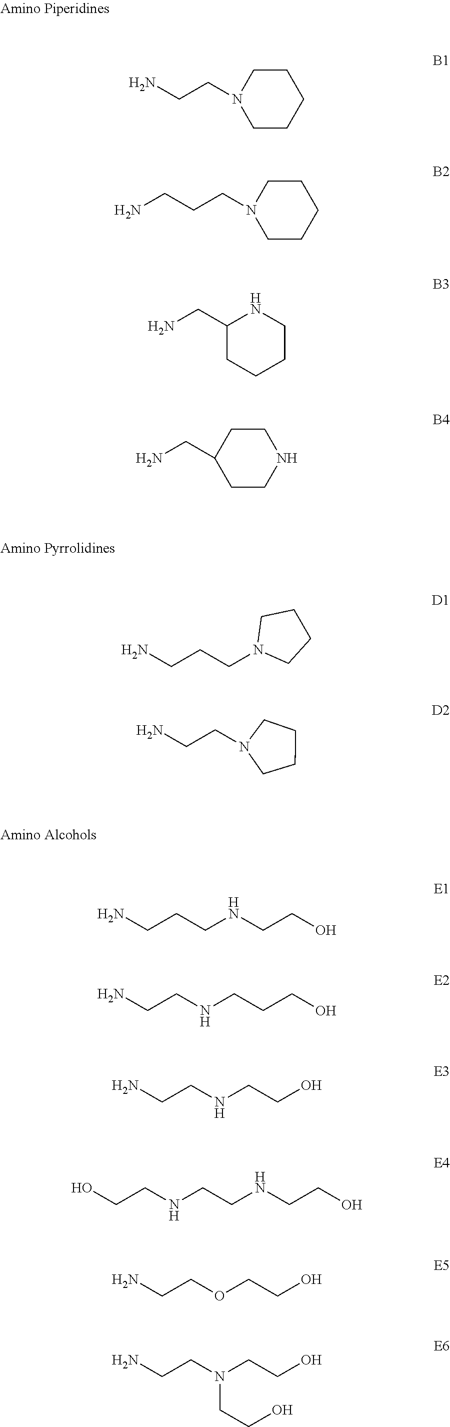

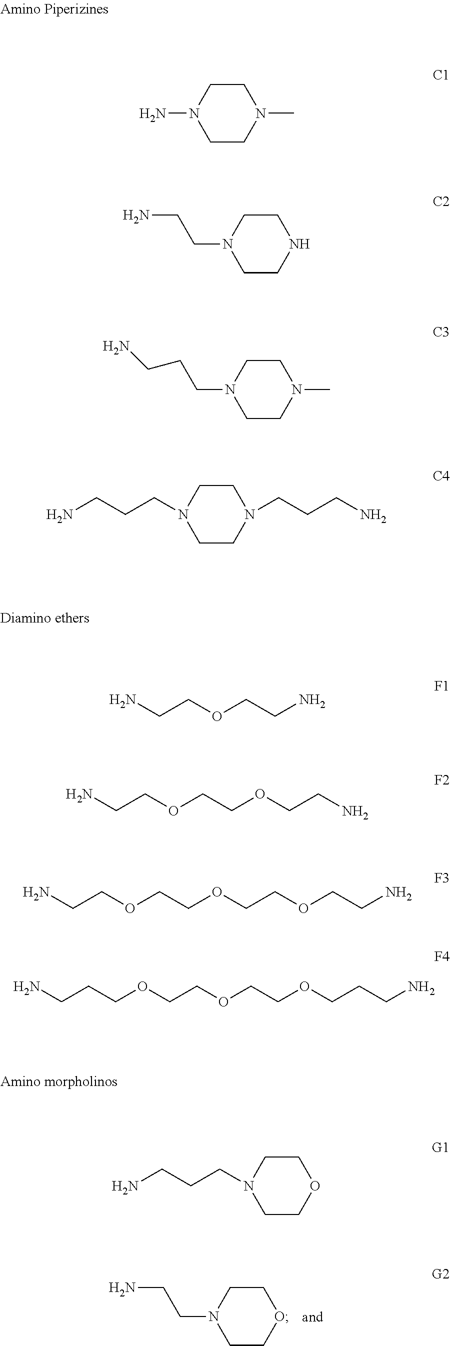

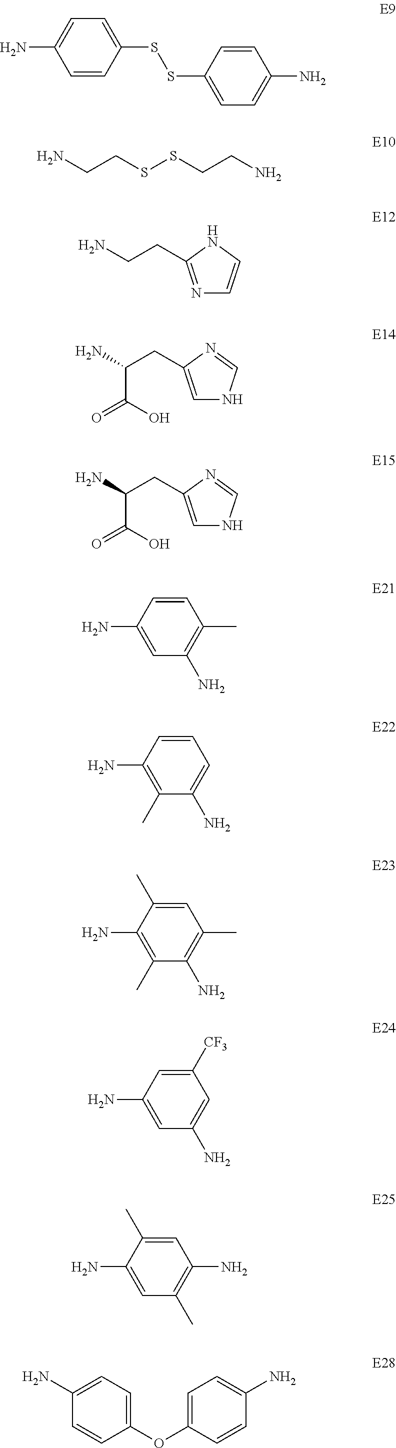

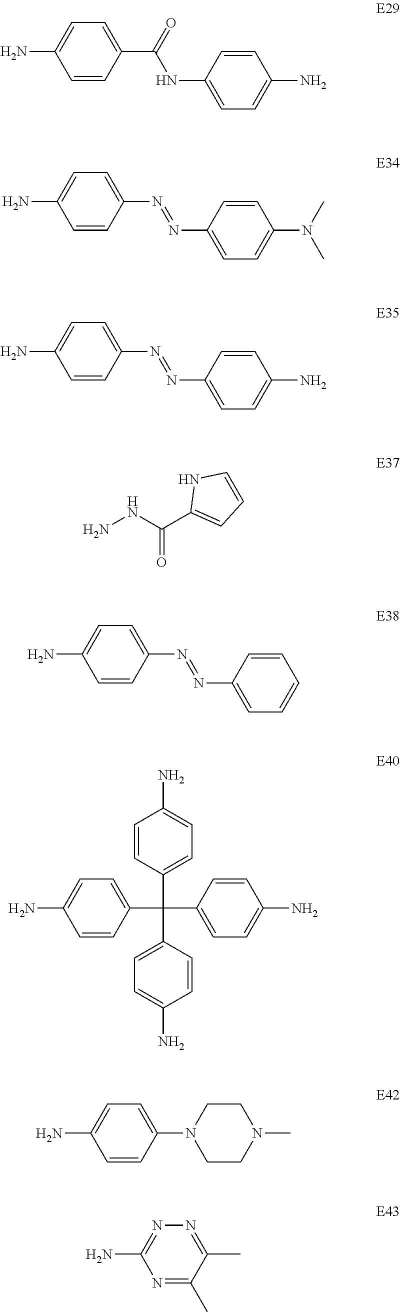

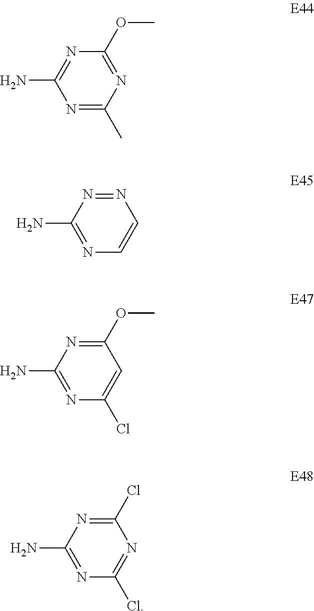

In some embodiments, —(CH₂)ₘ₁—(C═C)_{q}—(CH₂)ₘ₂—CH₃ is selected from the group consisting of:

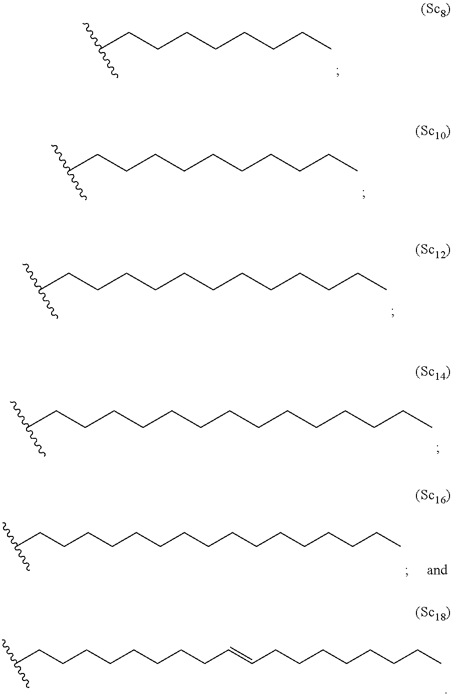

These components are derived from their amine-based monomers, e.g., 1-dodecylamine (Sc12), tetradecylamine (Sc14), hexadecylamine (Sc16); and oleylamine (Sc18).

In certain embodiments, R is:

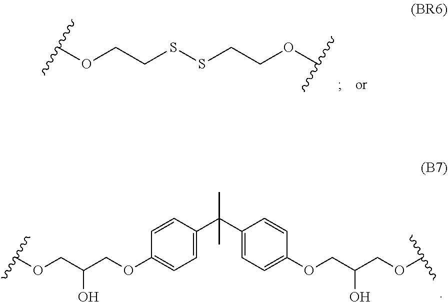 [SMILES: *OCCSSCCO* ; *OCC(O)COc1ccc(C(C)(C)c2ccc(OCC(O)CO*)cc2)cc1]

In certain embodiments, R′ is:

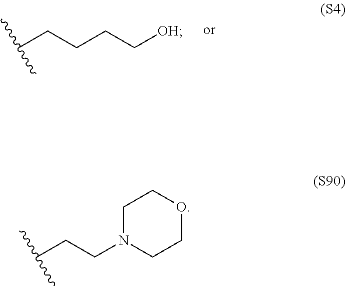 [SMILES: *CCCCO ; "or" ; *CCN1CCOCC1]

In certain embodiments, R″ is selected from the group consisting of:

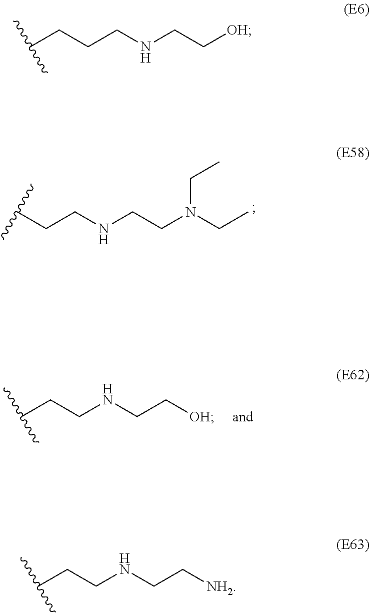 [SMILES: *CCCNCCO ; *CCNCCN(CC)CC ; *CCNCCO ; "and" ; *CCNCCN]

In certain embodiments, —(CH₂)ₘ₁—(C═C)_{q}—(CH₂)ₘ₂—CH₃ is selected from the group consisting of Sc12, Sc16, and Sc18.

In particular embodiments, the compound of formula (I) comprises:

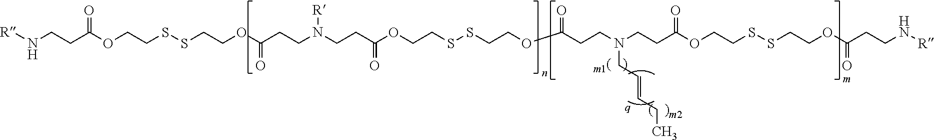

In particular embodiments, the compound of formula (I) comprises:

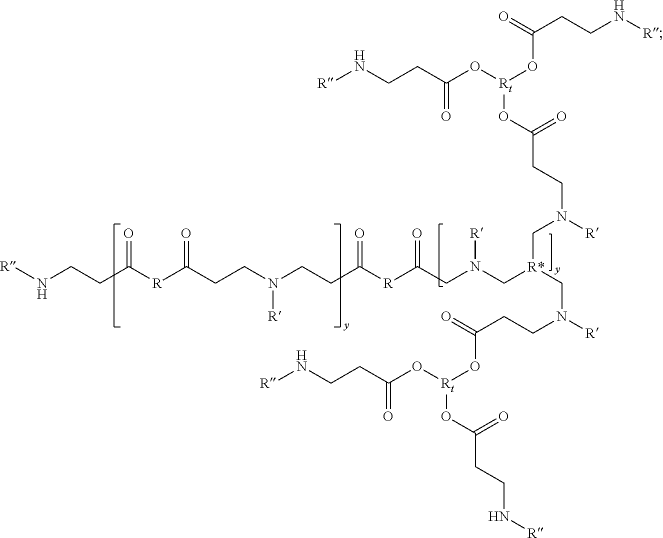

wherein each R* is a triacrylate, quanternary, or hexafunctional acrylate monomer selected from the group consisting of:

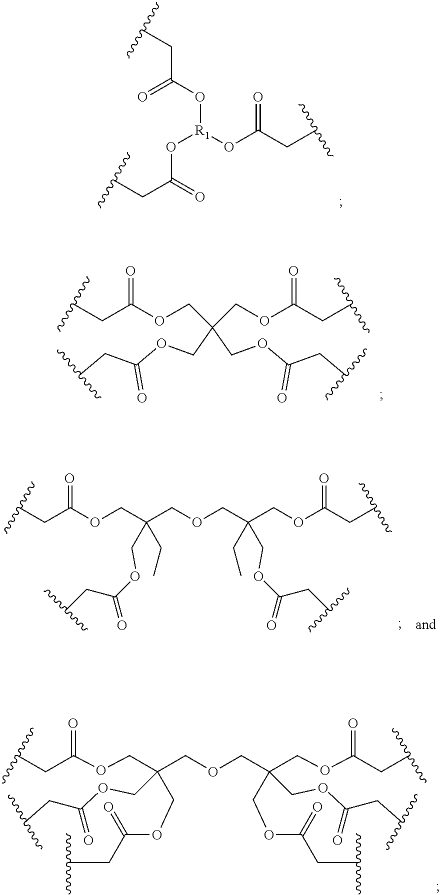

wherein each Rₜ is independently a trivalent group; and each y is independently an integer from 1 to 10,000.

In some embodiments, the composition has a ratio of the hydrophobic side chain to the hydrophilic side chain is between about 10:90 lipophilic side chain:hydrophilic side chain to about 90:10 lipophilic side chain:hydrophilic side chain, including 10:90, 15:85, 20:80, 25:75, 30:70, 35:65, 40:60, 45:55, 50:50, 55:45, 60:40, 65:35, 70:30, 75:25, 80:20, 85:15, and 90:10 lipophilic side chain:hydrophilic side chain.

In particular embodiments, the composition is selected from the group consisting of BR6-S4, Sc16-E6, 50%/50% ratio of S4/Sc16; BR6-S4, Sc16-E62, 50%/50% ratio of S4/Sc16; BR6-S4, Sc16-E63, 50%/50% ratio of S4/Sc16; BR6-S4, Sc18-E6, 50%/50% ratio of S4/Sc18; BR6-S4, Sc18-E62, 50%/50% ratio of S4/Sc18; and BR6-S4, Sc18-E63, 50%/50% ratio of S4/Sc18.

In other embodiments, the composition is selected from the group consisting of B7-S90, Sc12-E6, 50%/50% ratio of S90/Sc12; B7-S90, Sc12-E6, 20%/80% ratio of S90/Sc12; B7-S90, Sc12-E58, 50%/50% ratio of S90/Sc12; B7-S90, Sc12-E58, 20%/80% ratio of S90/Sc12; B7-S90, Sc12-E63, 50%/50% ratio of S90/Sc12; and B7-S90, Sc12-E63, 20%/80% ratio of S90/Sc12.

In certain embodiments, the composition comprises a weight ratio between the polymer and the nucleic acid from between about 30 w/w and about 200 w/w, including about 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, and 200 w/w. In particular embodiments, the weight ratio between the polymer and the nucleic acid is between about 50 w/w and about 150 w/w, including 50, 55, 60, 65, 75, 80, 85, 90, 95, 100, 105, 110, 115, 120, 125, 130, 135, 140, 145, and 150 w/w.

In certain embodiments, the one or more nucleic acids comprise DNA or RNA. In particular embodiments, the one or more nucleic acids are selected from the group consisting of an oligonucleotide, a cyclic dinucleotide, plasmid DNA, linear DNA, siRNA, miRNA, mRNA, and combinations thereof. In more particular embodiments, the one or more nucleic acids is mRNA. In certain embodiments, the mRNA comprises a self-amplifying mRNA (SAM).

In other embodiments, the composition further comprises one or more immunomodulatory nucleic acids. In certain embodiments, the one or more immunomodulatory nucleic acids are selected from the group consisting of CpG, GpG, poly(I:C), and a cyclic dinucleotide (CDN).

In some embodiments, the composition comprises a nanoparticle comprising a compound of formula (I) and one or more nucleic acids.

In some embodiments, the nanoparticle has at least one dimension in the range of about 50 nm to about 500 nm, or from about 50 to about 200 nm, including 50, 60, 70, 80, 90, 100, 110, 120, 130, 140, 150, 160, 170, 180, 190, and 200 nm. Exemplary nanoparticles may have an average size (e.g., average diameter) of about 50, about 75, about 100, about 125, about 150, about 200, about 250, about 300, about 400 or about 500 nm. In some embodiments, the nanoparticle has an average diameter of from about 50 nm to about 500 nm, from about 50 nm to about 300 nm, or from about 50 nm to about 200 nm, or from about 50 nm to about 150 nm, or from about 70 to 100 nm. In embodiments, the nanoparticle has an average diameter of from about 200 nm to about 500 nm. In embodiments, the nanoparticle has at least one dimension, e.g., average diameter, of about 50 to about 100 nm. Nanoparticles are usually desirable for in vivo applications. For example, a nanoparticle of less than about 200 nm will better distribute to target tissues in vivo.

In certain embodiments, the composition comprises a nanoparticle encapsulating an mRNA that encodes an autoreactive antigen. In particular embodiments, the autoreactive antigen comprises myelin oligodendrocyte glycoprotein (MOG). In certain embodiments, the composition further comprises GpG. In certain embodiments, the composition further comprises rapamycin.

In some embodiments, the composition further comprises lipid-PEG. In certain embodiments, the lipid-PEG is admixed with the compound of formula (I) at a varying mass percent. In particular embodiments, the lipid-PEG is selected from the group consisting of 1,2-dimyristoyl-rac-glycero-3-methoxypolyethylene glycol 2000 (DMG-PEG2k) and C18-PEG2k. In certain embodiments, the composition comprises DMG-PEG2k at a weight percent from about 2 wt % to about 10 wt %, including 2, 3, 4, 5, 6, 7, 8, 9, and 10 wt %. In particular embodiments, the composition further comprises a nanoparticle comprising a compound of formula (I), lipid-PEG, and one or more nucleic acids. In more particular embodiments, the nanoparticle has a zeta-potential that varies with a varying mass percent of lipid-PEG.

In some embodiments, the composition further comprises one or more excipients. In certain embodiments, the one or more excipients include one or more cryoprotectants, one or more sugar alcohols, MgCl₂, and combinations thereof. In more certain embodiments, the one or more cryoprotectants comprise a sugar. In particular embodiments, the sugar is selected from the group consisting of glucose, fructose, sorbitol, mannitol, sucrose, trehalose, and raffinose. In certain embodiments, the one or more sugar alcohols comprise sorbitol.

In some embodiments, the composition is lyophilized. In certain embodiments, the composition comprises a storable powder.

### B. Genetic Vaccines

In some embodiments, the presently disclosed subject matter provides a genetic vaccine comprising the presently disclosed compositions, wherein the genetic vaccine targets one or more antigen presenting cells. In some embodiments, the one or more nucleic acids comprises an mRNA encoding one or more antigens and/or one or more immunomodulatory nucleic acids selected from the group consisting of CpG, GpG, poly(I:C), and cyclic dinucleotides (CDN).

### C. Methods for Delivering One or More Nucleic Acids to a Subject

In some embodiments, the presently disclosed subject matter provides a method for delivering one or more nucleic acids to a subject, the method comprising administering a presently disclosed composition or presently disclosed genetic vaccine to the subject.

In certain embodiments, the administering comprises systemically administering the composition or the genetic vaccine to the subject. In particular embodiments, the method comprises intravenously administering the composition to the subject.

### D. Methods of Treatment

In some embodiments, the presently disclosed subject matter provides a method for treating a disease or condition, the method comprising administering a presently disclosed composition or the presently disclosed genetic vaccine to a subject in need of treatment thereof.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed composition of formula (I), to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

In certain embodiments, the disease or condition is selected from the group consisting of a cancer, an infectious disease, and an autoimmune disease.

In particular embodiments, the infectious disease is selected from the group consisting of a coronavirus, influenza, and rabies.

As used herein, a “cancer” in a patient refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells. A “tumor,” as used herein, refers to all neoplastic cell growth and proliferation, whether malignant or benign, and all precancerous and cancerous cells and tissues.

A “solid tumor,” as used herein, is an abnormal mass of tissue that generally does not contain cysts or liquid areas. A solid tumor may be in the brain, colon, breasts, prostate, liver, kidneys, lungs, esophagus, head and neck, ovaries, cervix, stomach, colon, rectum, bladder, uterus, testes, and pancreas, as non-limiting examples. In some embodiments, the solid tumor regresses or its growth is slowed or arrested after the solid tumor is treated with the presently disclosed methods. In other embodiments, the solid tumor is malignant. In some embodiments, the cancer comprises Stage 0 cancer. In some embodiments, the cancer comprises Stage I cancer. In some embodiments, the cancer comprises Stage II cancer. In some embodiments, the cancer comprises Stage III cancer. In some embodiments, the cancer comprises Stage IV cancer. In some embodiments, the cancer is refractory and/or metastatic. For example, the cancer may be refractory to treatment with radiotherapy, chemotherapy or monotreatment with immunotherapy. Cancer as used herein includes newly diagnosed or recurrent cancers, including without limitation, acute lymphoblastic leukemia, acute myelogenous leukemia, advanced soft tissue sarcoma, brain cancer, metastatic or aggressive breast cancer, breast carcinoma, bronchogenic carcinoma, choriocarcinoma, chronic myelocytic leukemia, colon carcinoma, colorectal carcinoma, Ewing's sarcoma, gastrointestinal tract carcinoma, glioma, glioblastoma multiforme, head and neck squamous cell carcinoma, hepatocellular carcinoma, Hodgkin's disease, intracranial ependymoblastoma, large bowel cancer, leukemia, liver cancer, lung carcinoma, Lewis lung carcinoma, lymphoma, malignant fibrous histiocytoma, a mammary tumor, melanoma, mesothelioma, neuroblastoma, osteosarcoma, ovarian cancer, pancreatic cancer, a pontine tumor, premenopausal breast cancer, prostate cancer, rhabdomyosarcoma, reticulum cell sarcoma, sarcoma, small cell lung cancer, a solid tumor, stomach cancer, testicular cancer, and uterine carcinoma.

In some embodiments, the cancer is acute leukemia. In some embodiments, the cancer is acute lymphoblastic leukemia. In some embodiments, the cancer is acute myelogenous leukemia. In some embodiments, the cancer is advanced soft tissue sarcoma. In some embodiments, the cancer is a brain cancer. In some embodiments, the cancer is breast cancer (e.g., metastatic or aggressive breast cancer). In some embodiments, the cancer is breast carcinoma. In some embodiments, the cancer is bronchogenic carcinoma. In some embodiments, the cancer is choriocarcinoma. In some embodiments, the cancer is chronic myelocytic leukemia. In some embodiments, the cancer is a colon carcinoma (e.g., adenocarcinoma). In some embodiments, the cancer is colorectal cancer (e.g., colorectal carcinoma). In some embodiments, the cancer is Ewing's sarcoma. In some embodiments, the cancer is gastrointestinal tract carcinoma. In some embodiments, the cancer is a glioma. In some embodiments, the cancer is glioblastoma multiforme. In some embodiments, the cancer is head and neck squamous cell carcinoma. In some embodiments, the cancer is hepatocellular carcinoma. In some embodiments, the cancer is Hodgkin's disease. In some embodiments, the cancer is intracranial ependymoblastoma. In some embodiments, the cancer is large bowel cancer. In some embodiments, the cancer is leukemia. In some embodiments, the cancer is liver cancer. In some embodiments, the cancer is lung cancer (e.g., lung carcinoma). In some embodiments, the cancer is Lewis lung carcinoma. In some embodiments, the cancer is lymphoma. In some embodiments, the cancer is malignant fibrous histiocytoma. In some embodiments, the cancer comprises a mammary tumor. In some embodiments, the cancer is melanoma. In some embodiments, the cancer is mesothelioma. In some embodiments, the cancer is neuroblastoma. In some embodiments, the cancer is osteosarcoma. In some embodiments, the cancer is ovarian cancer. In some embodiments, the cancer is pancreatic cancer. In some embodiments, the cancer comprises a pontine tumor. In some embodiments, the cancer is premenopausal breast cancer. In some embodiments, the cancer is prostate cancer. In some embodiments, the cancer is rhabdomyosarcoma. In some embodiments, the cancer is reticulum cell sarcoma. In some embodiments, the cancer is sarcoma. In some embodiments, the cancer is small cell lung cancer. In other embodiments, the cancer is non-small cell lung cancer. In some embodiments, the cancer comprises a solid tumor. In some embodiments, the cancer is stomach cancer. In some embodiments, the cancer is testicular cancer. In some embodiments, the cancer is uterine carcinoma.

In some embodiments, the cancer is selected from the group consisting of melanoma, non-small cell lung cancer, adrenocortical cancer, colon cancer, including refractory metastatic colon cancer, breast cancer, leukemia, osteosarcoma, medulloblastomas, and gliomas, such as glioblastoma multiforme and pediatric gliomas.

In particular embodiments, the cancer is selected from the group consisting of a solid tumor and a metastatic cancer.

In certain embodiments, the cancer comprises a solid tumor in one or more organs selected from the group consisting of the brain, colon, breast, prostate, liver, kidney, lung, esophagus, head and neck, ovaries, cervix, stomach, colon, rectum, bladder, uterus, testes, and pancreas.

In certain embodiments, the cancer comprises a metastatic cancer selected from the following types of cancer and metastasis sites: bladder: bone, liver, lung; breast: bone, brain, liver, lung; colon: liver, lung, peritoneum; kidney: adrenal gland, bone, brain, liver, lung; lung: adrenal gland, bone, brain, liver, other lung; melanoma: bone, brain, liver, lung, skin, muscle; ovary: liver, lung, peritoneum; pancreas: liver, 10 lung, peritoneum; prostate: adrenal gland, bone, liver, lung; rectal: liver, lung, peritoneum; stomach: liver, lung, peritoneum; thyroid: bone, liver, lung; and uterus: bone, liver, lung, peritoneum, vagina.

As used herein, the term “autoimmune disease” means a disease resulting from an immune response against a self-tissue or tissue component, including both self-antibody responses and cell-mediated responses. The term autoimmune disease, as used herein, encompasses organ-specific autoimmune diseases, in which an autoimmune response is directed against a single tissue, such as type I diabetes mellitus (T1D), Crohn's disease, ulcerative colitis, myasthenia gravis, vitiligo, Graves' disease, Hashimoto's disease, Addison's disease and autoimmune gastritis, autoimmune hepatitis, primary biliary cirrhosis, and autoimmune thrombocytopenia. The term autoimmune disease also encompasses non organ specific autoimmune diseases, in which an autoimmune response is directed against a component present in several or many organs throughout the body. Such autoimmune diseases include, for example, rheumatoid diseases, systemic lupus erythematosus, progressive systemic sclerosis and variants, polymyositis and dermatomyositis, inflammatory bowel disease, celiac disease, inflammatory myositis, Sjogren's syndrome, multiple sclerosis, psoriasis and scleroderma.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In particular embodiments, the subject is a human or an animal. In yet more particular embodiments, the method is selected from a prophylactic treatment method, a therapeutic treatment method, and combinations thereof.

### E. Kits

In some embodiments, the presently disclosed subject matter provides a kit, the kit comprising one or more of: one or more compounds of formula (I), one or more nucleic acids, reagents, and instructions for use.

In certain embodiments, the disclosed kits comprise one or more containers, including, but not limited to a vial, tube, ampule, bottle and the like, for containing the pharmaceutical composition including one or more compounds of formula (I). The compounds of formula (I) may be solvated, in suspension, or powder form, and may then be reconstituted in the pharmaceutically acceptable carrier to provide the pharmaceutical composition. The one or more containers also can be carried within a suitable carrier, such as a box, carton, tube or the like. Such containers can be made of plastic, glass, laminated paper, metal foil, or other materials suitable for holding medicaments.

In certain embodiments, the container can hold a pharmaceutical composition and may have a sterile access port (for example the container may be an intravenous solution bag or a vial having a stopper pierceable by a hypodermic injection needle). Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific Examples that follow are only intended for the purposes of illustration, and are not to be construed as limiting in any manner to make compounds of the disclosure by other methods.

### Example 1

### Synthesis of Compounds of Formula (I)

As provided in Scheme 1 immediately herein below, the presently disclosed compounds of formula (I) can be prepared by condensing a bioreducible acrylate backbone monomer with lipophilic and hydrophilic amine-containing side chain monomers, then polymerizing and end capping with an amine end capping group to form a compound of Formula 1.

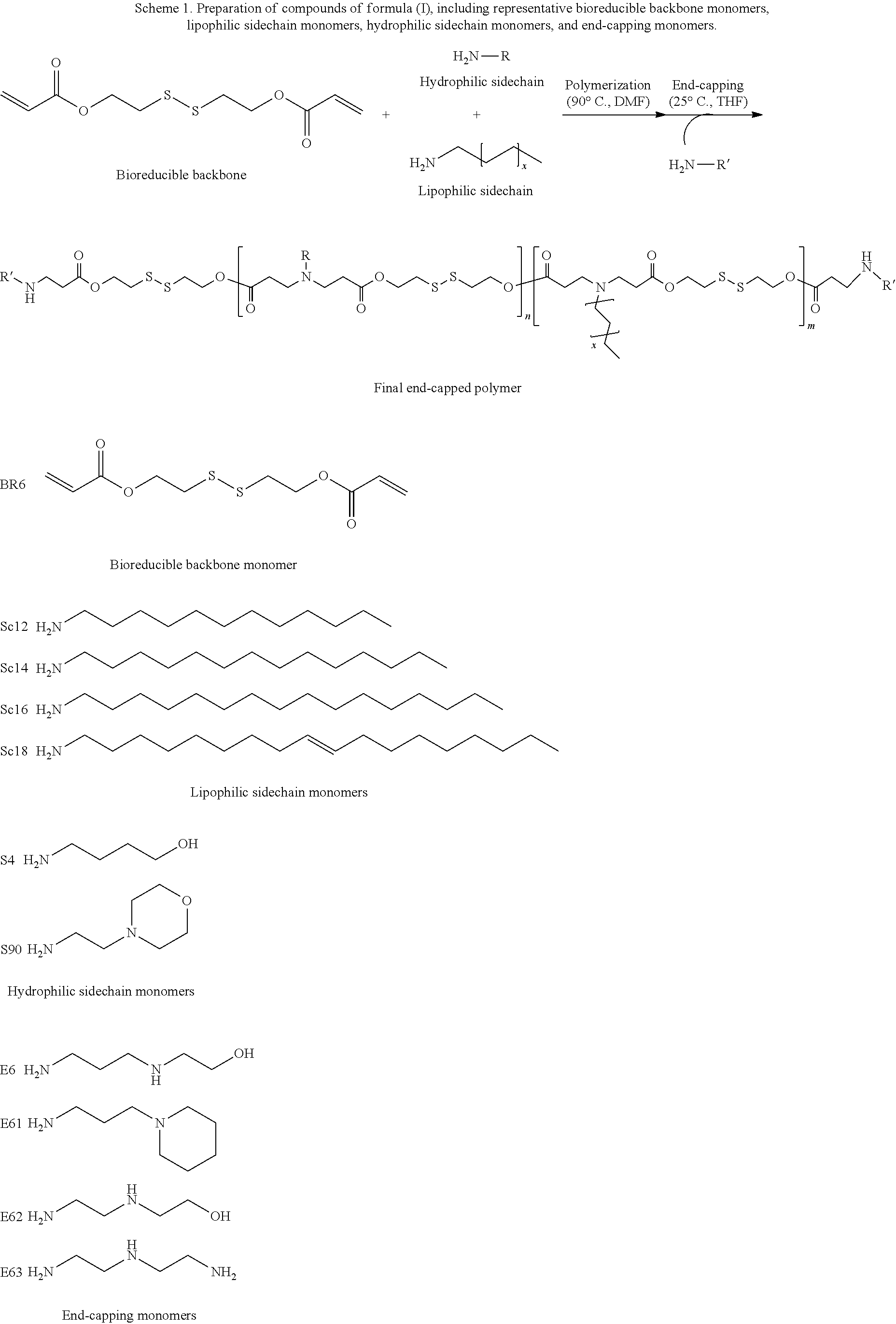

### Example 2

Systemic mRNA Delivery with Biodegradable Lipophilic Polymeric Nanoparticles Enables Ligand-Free Targeting of Splenic Dendritic Cells for Cancer Vaccination

### 2.1 Overview

Nanoparticle-based messenger RNA cancer vaccines hold great promise to realize personalized cancer treatments that can be potentially low-cost and broadly accessible. Potent antitumor immunity requires safe, targeted, and efficient intracellular mRNA delivery and activation of dendritic cells for inducing cancer-specific antigen expression and activation antigen-specific T cells. Here, we developed a class of bioreducible lipophilic poly(beta-amino ester) nanocarriers with quadpolymer architecture. In this strategy, nanoparticles are formed in one-step via self-assembly, making the platform agnostic to the mRNA sequence and allowing for delivery of multiple antigen-encoding mRNAs as well as co-delivery of nucleic acid-based adjuvants. We synthesized a combinatorial polymer library and evaluated the intracellular mRNA delivery efficiency in dendritic cells to elucidate structure-function relationships. The lipid subunit of the polymer structure was critical for efficient intracellular mRNA delivery to dendritic cells and for encapsulation of mRNA and adjuvant molecules in the presence of serum. Following systemic administration, the engineered nanoparticles facilitated targeted delivery to the spleen and preferential transfection of dendritic cells without the need for surface functionalization or ligand-directed targeting. Treatment with engineered nanoparticles co-delivering antigen-encoding mRNA and toll-like receptor agonist adjuvants led to robust antigen-specific CD8+ T-cell responses, resulting in efficient antitumor therapy in in vivo models of murine melanoma and colon adenocarcinoma.

### 2.2 Background

In recent years, the field of immunotherapy and the use of immune-checkpoint inhibitors has revolutionized treatment of many cancers and led to approvals by the FDA of novel therapeutics for various tumor types. Blass and Ott, 2021; Whiteside et al., 2016. However, the diversity of tumor epitopes between individuals makes each patient's tumor unique; thus, the development of personalized cancer therapy technologies would add an important tool to the immunotherapy toolbox. Vormehr et al., 2019. The availability of personalized diagnostics using sequencing and bioinformatics technologies have made it possible in a timely and cost-effective manner to identify tumor-specific mutations, referred as neoantigens, in individual patients. Wang and Wang, 2017; Hu et al., 2018. A promising strategy to target these neoantigens that are exclusively expressed by cancer cells is delivery of neoantigen-encoded mRNA. In this approach, neoantigen mRNA needs to be delivered to antigen presenting cells (APCs), such as dendritic cells (DCs), to induce a tumor-specific T-cell response. mRNA vaccine technologies offer many benefits over conventional vaccine approaches, including high potency, potential for low-cost manufacturing, capacity for rapid development, and improved safety. Pardi et al., 2018. Key advantages of mRNA-based vaccine technologies compared to live-attenuated and inactivated vaccines are that they carry no risk of genome integration or infection and are highly modular, so they can easily be adapted for various proteins of interest. Sahin et al., 2014; Kaczmarek et al., 2017.

The main challenge to realize the potential of mRNA cancer vaccines is the requirement for delivery materials to protect the mRNA molecules from extracellular degradation by RNases and facilitate efficient intracellular mRNA delivery to DCs. The use of lipid nanoparticles (LNPs) for delivery of mRNA encoding the spike protein of the SARS-CoV-2 virus has provided safe and powerful vaccine technologies against COVID-19, Polack et al., 2020; Baden et al., 2021; Shin et al., 2020, demonstrating the great potential and increasing public acceptance of mRNA vaccines. In these prophylactic vaccine platforms, mRNA-LNPs are delivered intramuscularly (i.m.) to induce immunity, with a focus on humoral immunity. To broaden this technology for therapeutic cancer vaccines, delivery technologies facilitating systemic delivery to APCs would be preferred to maximize cellular immunity, as expression of antigen by APCs will allow antigen presentation on class I major histocompatibility complex (MHC) molecules, crucial for the production of antigen-specific cytotoxic CD8+ T cells. In a systemic approach, cell target-specific mRNA delivery is desirable both for efficacy and to minimize any risks of systemic side effects. Kim et al., 2021. The use of RNA therapeutics for systemic delivery had a major breakthrough in 2018 when the first RNA interference technology using a LNP formulation for siRNA delivery received its first FDA approval. Adams et al., 2018; Akinc et al., 2019. This formulation facilitated transfection in hepatocytes as treatment for a liver disorder (polyneuropathies). However, current delivery technologies for RNA therapeutics have had limited success to facilitate extrahepatic transfection. Thus, new nanoparticle (NP) formulations are needed to achieve safe, effective, and specific transfection in organs beyond the liver following systemic administration.

Biodegradable polymeric NPs represent a promising class of delivery vehicles for clinical application, since they offer scalable production, wide flexibility in cargo encapsulation, and high safety. Karlsson et al., 2018. Accordingly, the possibility of synthesizing them with diverse chemistries makes them attractive for delivery beyond the liver. Additionally, polymers can include several subunits in the same molecule and thereby attain several orthogonal functionalities simultaneously. A single polymer structure can thus be designed for efficient intracellular trafficking and endosomal escape followed by quick release of RNA into the cytosol. Poly(beta-amino ester)s (PBAE)s are biodegradable cationic polymers that spontaneously self-assemble with anionic nucleic acids into NPs in aqueous solutions. PBAEs have several advantageous characteristics, including positive charge for efficient binding of RNA therapeutics, Lopez-Bertoni et al., 2018; Karlsson et al., 2019a, high buffering capacity in an acidic environment for endosomal escape, Rui et al., 2022; Sunshine et al., 2012, and hydrolytic degradation into nontoxic byproducts under aqueous conditions, that make them a promising delivery material to bring RNA therapeutics into clinical applications. Karlsson et al., 2020. Moreover, in previous studies using combinatorial library synthesis, we have identified PBAE structures for preferential delivery of siRNA and DNA to various cell types. Kozielski et al., 2014; Zamboni et al., 2017; Sunshine et al., 2009; Tzeng et al., 2011. PBAEs have also shown promise for mRNA delivery; Patel et al. reported the design of hyperbranched PBAE NPs, which facilitated efficient transfection of lung epithelial cells following nebulized administration, Patel et al., 2019, and a recent report demonstrates the capacity of PBAE-mediated delivery of mRNA to non-liver and non-lung targets. Rui et al., 2022. However, for cancer vaccine applications, new polymer architectures of PBAE nanocarriers are needed for efficient systemic delivery and transfection targeted specially to DCs.

In addition to DC transfection of the antigen-encoding mRNA, DC activation is required to boost the immune response for potent cancer treatment. One of the most promising classes of adjuvants for inducing T cell immunity are toll-like receptor (TLR) agonists, which stimulate DCs and induce expression of co-stimulatory molecules and secretion of cytokines that drive T-cell responses. Iwasaki and Medzhitov, 2004; Maisonneuve et al., 2014. TLR adjuvants of interest for cancer vaccine formulations include the TLR3 agonist polyinosinic-polycytidylic acid (poly(I:C)) and the TLR9 agonist CpG oligodeoxynucleotide (CpG ODN). In addition to being a TLR3 agonist, poly(I:C) can also activate cytosolic RIG-1, Gale et al., 2020, inducing an interferon response and leading to activation cytotoxic T-cells. As the self-assembly of PBAE NPs is based on electrostatic interactions, mRNA and anionic nucleic acid-based adjuvants can be co-encapsulated and co-delivered for DC activation. This strategy ensures that each transfected cell also becomes activated by the adjuvant. Additionally, the controlled biodistribution and cellular uptake provided by NP-mediated delivery is beneficial for maximized activation by the adjuvant; thus, a lower adjuvant dose is required compared to methods that rely on unassisted uptake of the adjuvant, which reduces the risk of systemic side effects, such as cytokine storm. Roth et al., 2021. Despite the importance of DC stimulation, nonspecific activation by the mRNA cargo may upregulate protein kinase R, which inhibits antigen expression and leads to reduced antitumor immune response. Pardi et al., 2018. To reduce the immunogenicity of mRNA, chemically modified nucleotides or mRNA molecules with optimized codon sequences have been developed. Karikó et al., 2008. Additionally, the adjuvant dose needs to be fine-tuned in co-delivery technologies, since a higher activation by the specific TLR agonist may also result in decreased protein translation of the antigen-encoded mRNA. Pastor et al., 2018. An ideal delivery technology would also allow for co-delivery of multiple antigen-encoding mRNAs, which likely is needed to completely eradicate tumors, Verdegaal et al., 2016, since they generally have a high degree of genetic heterogeneity. Dagogo-Jack and Shaw, 2018. Additionally, mRNA cancer vaccine technologies may be used in combination with clinically approved and emerging immunotherapy treatments, such as immune-checkpoint blockade, for synergistic effect. It was reported in a clinical study treating patients with stage IV melanoma that the antitumor T-cell response was broadened with combinational treatment of a T-cell induced vaccine and the immune checkpoint inhibitor pembrolizumab (anti-PD-1). Ott et al., 2017. Their synergistic effects might also be beneficial in preventing T-cell exhaustion, which is a major hurdle to eliciting potent antitumor immunity. McLane et al., 2019.

### 2.3 Scope

Here, we present a structural design of bioreducible lipophilic PBAE NPs that address the major challenges for mRNA cancer vaccines by enabling efficient intracellular mRNA delivery to DCs following systemic administration. We used combinatorial library synthesis to generate polymeric nanocarriers and demonstrated that both the incorporation of lipid subunit and endcap modifications of the polymer structure tuned intracellular trafficking in DCs. Additionally, we incorporated disulfide bonds in the backbone structure for environmentally triggered biodegradation upon entry into the reducing environment of the cytosol. Meng et al., 2009; Manickam et al., 2010; Karlsson et al., 2019b. We performed high-throughput in vitro screens in both DC2.4 cells (immortalized murine DCs) and murine bone-marrow-derived dendritic cells (BMDCs) to examine structure-function relationships, which identified the lipid subunit as a key structural component. The leading bioreducible lipophilic PBAE NP formulation facilitated effective in vitro transfection of DC2.4 cells at extremely low mRNA doses, which makes it a promising platform for co-delivery of multiple neoantigen-encoding mRNAs. Moreover, we demonstrated that the engineered bioreducible lipophilic PBAE NPs facilitated tissue-specific transfection in the spleen without the need for any surface functionalization, such as PEGylation or ligand-mediated uptake. Suk et al., 2016. We further utilized the transgenic Ai9 mouse model to sensitively detect individual transfected cells, which demonstrated that the engineered NPs facilitated preferential delivery to splenic DCs. The leading bioreducible lipophilic PBAE NP formulation for DC transfection was further examined for therapeutic efficacy using in vivo murine tumor models, in which the inoculated melanoma cells (B16) or colon adenocarcinoma cells (MC38) expressed the model antigen ovalbumin (OVA). In addition to carrying OVA-mRNA to achieve a cancer-specific immune response, the NPs also co-delivered either CpG ODN or poly(I:C) to evaluate how the antitumor response could be boosted. Systemically administered bioreducible lipophilic PBAE NPs facilitated a robust antigen-specific CD8+ T-cell response for antitumor treatment. In another in vivo non-OVA-expressing mouse model, a melanoma model using B16-F10, we demonstrated that this NP design also achieved antitumor response when delivering antigen-mRNAs encoding endogenous melanoma-associated antigens.

### 2.4 Results

### 2.4.1 Design, Synthesis, and Characterization of Polymeric NPs

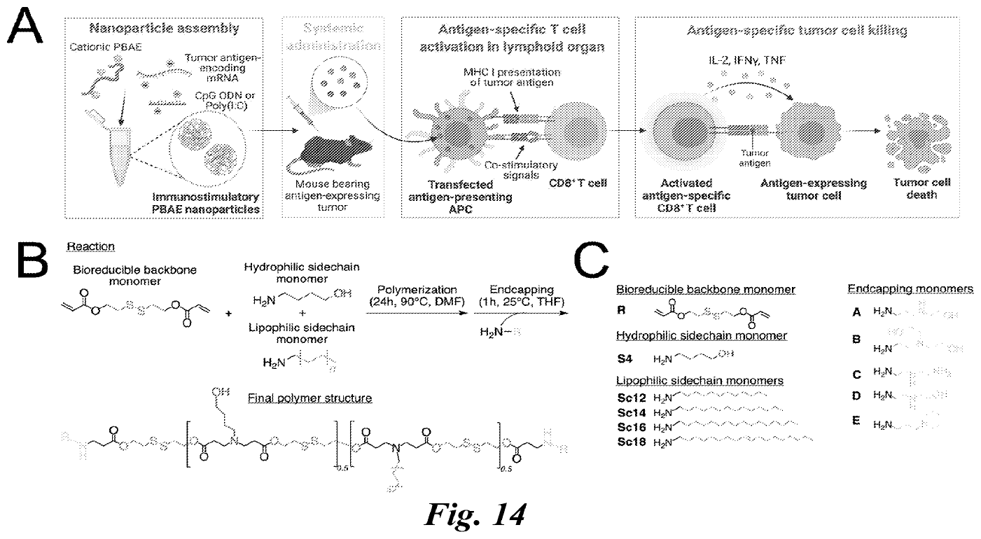

We engineered a polymeric NP platform composed of PBAEs to facilitate efficient and targeted mRNA delivery to DCs following systemic administration and explored its potential as an mRNA-based cancer vaccine (FIG. 14A). These PBAE NPs are formed by self-assembly through cationic amine groups in the polymer structure that electrostatically bind anionic nucleic acid therapeutics to spontaneously form nanoscale particles under mildly acidic aqueous conditions (pH=5.0). Karlsson et al., 2020. A major advantage of using polymeric nanocarriers is that they can include several functionalities in the same molecule through varied chemistry and arrangement of monomer units. Kim et al., 2021; Karlsson et al., 2018. We used combinatorial library synthesis to create a polymeric nanocarrier that would facilitate efficient intracellular trafficking, including cellular uptake, endosomal escape, and cytosolic cargo release. We have previously demonstrated both in vitro and in vivo that PBAE nanocarriers can be engineered for cell-type specific delivery of nucleic acid therapeutics, including DNA and siRNA, to cancer cells. Kozielski et al., 2014; Zamboni et al., 2017; Karlsson et al., 2021b. Additionally, previous work has found that small changes in the monomer structures can dramatically impact DNA transfection efficiency and specificity, Lopez-Bertoni et al., 2018, due in part to changes in cellular uptake pathways, Harayama and Riezman, 2018, motivating the use of a library screening approach to identify optimal polymer structures. In this study, our objective was to design PBAE nanocarriers that achieve potent and preferential mRNA delivery to DCs. In a two-step synthesis using Michael addition, we combined diacrylate backbone, amine side-chain, and endcap monomers to form the final PBAE structure (FIG. 14B). In addition to the biodegradable ester bonds in the backbone structure, we also incorporated disulfide bonds to create bioreducible (R) polymers to enable environmentally-triggered rapid degradation in the reducing environment of the cytosol, which also increases the safety of the polymers (FIG. 14C). Lopez-Bertoni et al., 2018; Mitchell et al., 2020. The high concentration of glutathione (GSH) in the cytosol (approximately 10 mM GSH) readily cleaves disulfide bonds upon entry to the cytosol, leading to triggered release of encapsulated biomolecules. Meng et al., 2009; Blanco et al., 2015. As side-chains, we combined both hydrophilic (S4) and lipophilic monomers (referred to as ScX, where X is the number of carbons in the alkyl tail) to create amphipathic polymers. We examined the use of monomers with varied alkyl length as a lipophilic subunit to identify chemical compositions for optimal interactions with the cellular membrane of DCs. Harayama and Riezman, 2018. Additionally, the use of a lipophilic subunit may improve the encapsulation of mRNA through hydrophobic interactions in addition to electrostatic interactions. As small molecular polymer-endcaps, we used amine-modified monomers in which we varied the position of the amine groups and the degree of hydroxyl groups to explore how endcap chemical structure influences cellular uptake and endosomal escape.

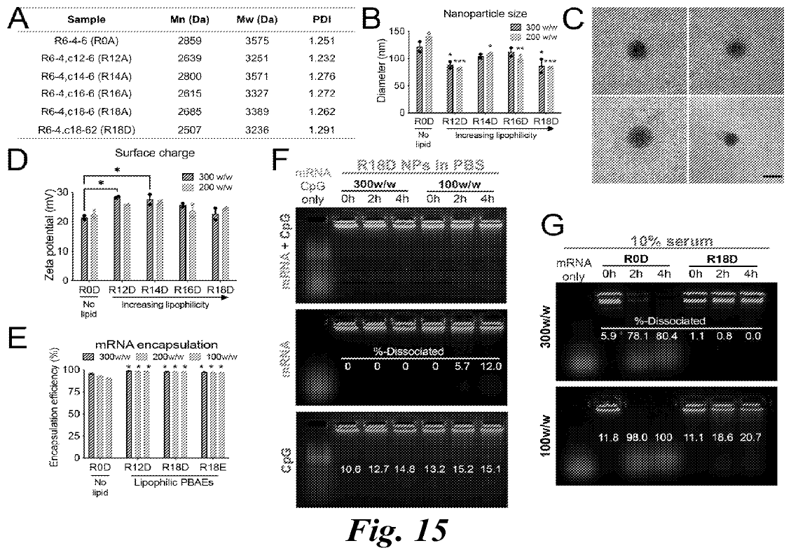

We assessed the polymer molecular weights using gel permeation chromatography (GPC), and they remained approximately the same (in the range of 2.5-2.8 kDa) after changing either the lipophilicity (R0A through R18A) or the endcap molecule (R18A compared to R18D) (FIG. 15A). We used dynamic light scattering (DLS) to analyze NP size as a function of lipophilicity (FIG. 15B). The incorporation of a lipid subunit decreased the hydrodynamic diameter of the NPs from 140±6 nm for ROD NPs (no lipid; 200 w/w) down to 81±1 nm for R12D NPs (Sc12 lipid; 200 w/w), likely due to increased hydrophobic interactions with the mRNA cargo forming more condensed NPs. However, increasing the length of the alkyl side-chain of the lipophilic subunit didn't decrease the NP size further. Similarly, changing the weight ratio (w/w) of polymer to mRNA from 300 w/w to 200 w/w did not influence the NP size. We performed transmission electron microscopy (TEM) to visualize mRNA-NPs using R18D as the nanocarrier, which verified that the PBAEs form spherical mRNA NPs of approximately 100 nm in diameter (FIG. 15C). Using our approach of administering the engineered NPs systemically to induce high protein production of the delivered antigen-encoding mRNA, we chose to design NPs with size larger than approximately 10 nm to avoid clearance through the kidneys and smaller than approximately 200 nm to avoid rapid clearance from circulation by the reticuloendothelial system. Wang et al., 2019.

We performed zeta potential measurements and found that all NPs of varying lipophilicity are cationic, with a surface charge in the range of +20 to +30 mV. The surface charge was statistically higher for lipophilic NP formulations using R12D and R14D nanocarriers compared to the non-lipophilic formulation using ROD (FIG. 15D) (*P<0.05; n=2). The higher surface charge is likely due to the lipophilic subunit contributing to hydrophobic interactions with the mRNA, exposing more of the cationic charge of the polymer at the NP surface. Moreover, using the Ribogreen assay we observed improved mRNA encapsulation for the lipophilic PBAE NPs compared to NPs with no lipid subunit (*P<0.05; n=3). The mRNA encapsulation efficiency for lipophilic PBAE NPs was above 97% even for formulations with lower polymer/mRNA ratios.

In addition to efficiently presenting the tumor antigen, DCs need to become activated to induce a robust cytotoxic T-cell response against the cancer cells. Hu et al., 2018. Our cationic nanocarriers can be used for co-delivery of antigen-encoding mRNA and nucleic acid-based adjuvants. Accordingly, we evaluated the ability of the bioreducible lipophilic PBAE nanocarrier R18D to co-encapsulate mRNA and the nucleic acid adjuvant CpG over time using a gel electrophoresis assay. We prepared R18D NPs carrying fluorescently labeled mRNA and CpG at 300 w/w and 100 w/w and incubated them in PBS over 4 h. The majority of the mRNA and CpG doses remained encapsulated in the R18D NPs, indicating that they are stably bound in the particles. The highest observed dissociation was 12.0% and 15.2% for mRNA and CpG, respectively (FIG. 15F).

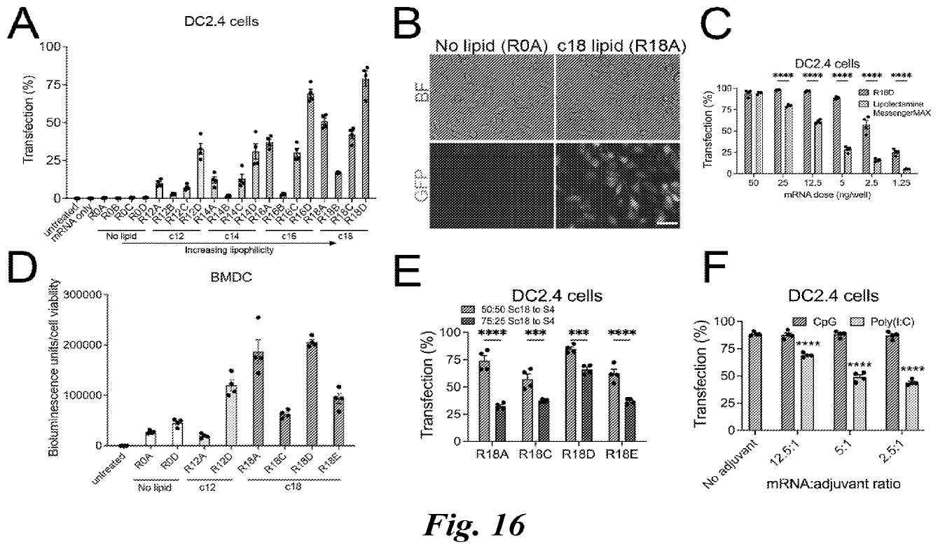

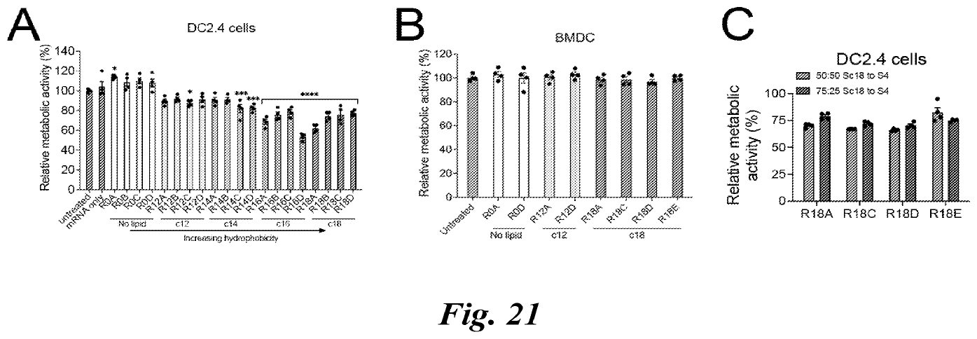

Previous electrostatically formed NP formulations composed of cationic polymeric nanocarriers have had limited success following systemic administration mainly due to insufficient stability in the presence of anionic serum proteins that readily dissociate the formulations prior to reaching the targeted site. Blanco et al., 2015; Wang et al., 2019. In addition to electrostatic interactions, the NP design using lipophilic PBAEs as nanocarriers facilitates hydrophobic interactions with the therapeutic nucleic acid cargo. To examine whether the presence of the lipophilic subunit would influence NP stability, we performed a gel electrophoresis assay, in which the NPs were incubated in media with 10% serum over 4 h (FIG. 15G). The PBAE NPs without a lipophilic subunit (ROD NPs) completely dissociated (100% release) when formed at 100 w/w, and 80% of the mRNA load was released over 4 h for the 300 w/w formulation. On the other hand, mRNA within lipophilic R18D NPs remained securely encapsulated in the presence of serum. Even when formulated at a lower polymer/mRNA ratio of 100 w/w, only 21% of mRNA was released over 4 h. 2.4.2 In vitro evaluation of mRNA delivery to DCs We evaluated the PBAE NP library for transfection efficiency and toxicity using the murine DC2.4 cell line and primary murine BMDCs to identify the top-performing PBAE structure and examine the roles of polymer hydrophobicity and endcapping chemistry. In these high-throughput in vitro screens, NPs delivered eGFP mRNA, and transfection was assessed quantitatively by flow cytometry (FIG. 16A) and qualitatively by fluorescence microscopy (FIG. 16B) 24 hours post-treatment. We also evaluated cell viability using the MTS assay 24 hours post-treatment (FIG. 21A). The percent of cells positively transfected generally increased as polymer lipophilicity increased, with non-lipophilic NPs (R0A-D) providing little to no transfection (FIG. 16A-FIG. 16B). In each lipophilic PBAE series (R12, R14, R16 and R18), the PBAE with the D endcap monomer facilitated the highest level of transfection (FIG. 16A). The most lipophilic of those, R18D, achieved the highest level of transfection and was, therefore, selected for further studies. Structures with the B endcap monomer showed very low transfection efficacy, indicating that the secondary amine in the A, C, and D endcaps is preferable to the tertiary amine and additional hydroxyl group in the B endcap. These studies also highlight the utility of evaluating a polymer library with differential structure for mRNA delivery, as small, seemingly minor changes to single atoms in the side chain or end-group of the polymer lead to dramatic functional differences in the transfection of DCs.

The top-performing structure, R18D, was further investigated in DC2.4 cells at a wide range of mRNA doses and compared to Lipofectamine MessengerMAX, a leading commercial mRNA transfection agent for hard-to-transfect cells (FIG. 16C). R18D NPs performed significantly better than Lipofectamine MessengerMAX at all but the highest mRNA dose and transfected nearly 100% of cells at the top three mRNA doses. Even at a low dose of 5 ng mRNA per well in a 96-well plate (50 ng mRNA/mL), 20 times lower than what is traditionally recommended for commercial transfection reagents, R18D NPs transfected 88.6% of cells on average, while Lipofectamine MessengerMAX only transfected 28.7% of cells on average.

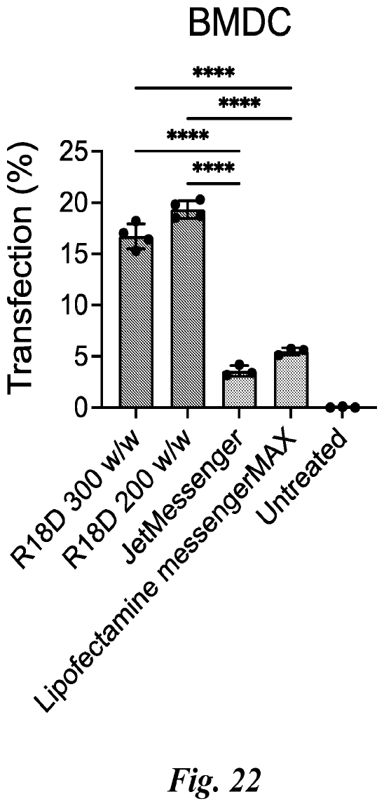

To better mimic in vivo DCs, we evaluated a subset of the PBAE NP library on primary murine BMDCs. In this screen, tested NP formulations delivered fLuc mRNA and bioluminescence was assessed after 24 hours to evaluate relative transfection (FIG. 16D). Similar to the DC2.4 cells, BMDCs showed the highest level of transfection with the most lipophilic NPs, and R18D was a top-performing candidate. Importantly, the NPs caused no toxicity in the BMDCs (FIG. 21B). We also compared R18D NPs to two leading commercial mRNA transfection reagents in BMDCs (FIG. 22). R18D NPs formed at 200 and 300 w/w led to significantly higher transfection of eGFP mRNA compared to both commercial reagents, transfecting approximately 17-20% of BMDCs, compared to Lipofectamine MessengerMAX and jetMESSENGER, which transfected 3.6% and 5.5% of BMDCs, respectively.

To further examine the effect of PBAE hydrophobicity on transfection efficiency, we synthesized PBAEs in the R18 series with a 75:25 molar ratio of lipophilic Sc18 monomer to hydrophilic S4 monomer and compared their in vitro efficacy to the previously used PBAEs synthesized with a 50:50 molar ratio of the two monomers (FIG. 16E). All PBAE NPs with a 50:50 ratio of Sc18 to S4 performed significantly better than their 75:25 counterparts with no differences in cell viability (FIG. 21C), suggesting that, past a certain point, increased backbone hydrophobicity causes a loss in DC transfection efficacy. Finally, we examined how co-delivery of nucleic-acid based adjuvants CpG ODN or poly(I:C) affects R18D mRNA NP transfection efficacy in DC2.4 cells, as increased immunogenicity may reduce transfection (FIG. 16F). Bessis et al., 2014. Co-delivery of CpG did not influence mRNA transfection, whereas poly(I:C) did significantly reduce transfection in a dose-dependent fashion.

2.4.3 Effect of polymer structure on NP-mediated intracellular trafficking

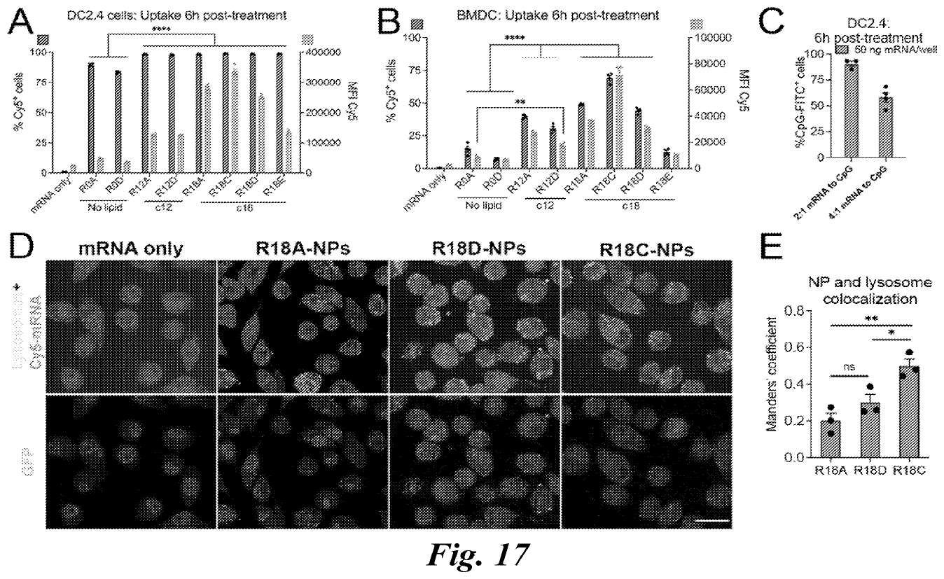

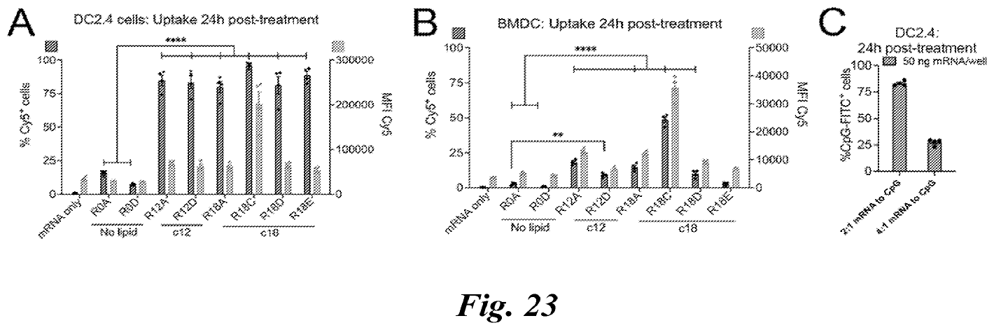

In the in vitro transfection experiments, we demonstrated that the bioreducible lipophilic PBAE NPs facilitated efficient intracellular delivery of mRNA in both immortalized (DC2.4) and primary murine DCs (BMDC). In subsequent experiments, we used fluorescently labeled NPs to facilitate mechanistic understanding of how the structural design of the nanocarrier influences intracellular trafficking. We first analyzed cellular uptake in DC2.4 cells using NPs carrying Cy5-labeled mRNA. All tested PBAE NPs were taken up by over 83% of cells, with the lipophilic NPs demonstrating cellular uptake between 97% and 99% after 6 h (FIG. 17A). We also analyzed the mean fluorescence intensity of Cy5, which correlates with the average number of NPs taken up per cell, and found that NPs with the longer alkyl side-chain (Sc18) were taken up more efficiently than NPs with a shorter alkyl side-chain (Sc12) (FIG. 17A). Both percent cellular uptake and fluorescence intensity of uptake were statistically higher for all lipophilic NPs compared to the NP formulations without a lipophilic subunit (****P<0.0001; n=4). We further examined NP uptake in BMDCs and found that increased lipophilicity of the nanocarrier improved cellular uptake (FIG. 17B). The NP uptake was statistically higher for the lipophilic NPs compared to NPs without a lipophilic subunit using the same endcap (R12A and R18A compared to R0A; R12D and R18D compared to ROD; ****P<0.0001; n=4). This finding demonstrates that the chemical structure of the lipophilic subunit is a key property for cellular uptake, indicating that the chemistry of the lipid subunit influences the interactions with the cellular membrane of DCs. The trend of improved 20 cellular uptake with increased lipophilicity supports the higher in vitro transfection efficiency observed for lipophilic mRNA-NPs (FIG. 16A, FIG. 16D). Additionally, endcap modifications also influenced the cellular uptake, as shown for NP formulations using Sc18 as the lipophilic subunit, where the nanocarrier synthesized with endcapping monomer C (R18C) facilitated the highest uptake (FIG. 17A-FIG. 17B). The same trends between nanocarrier structure and NP uptake for cellular uptake at 6 h post-treatment was also observed at 24 h post-treatment (FIG. 23A-23B). Moreover, we prepared R18D NPs co-encapsulating mRNA and FITC-labeled CpG adjuvant to assess cellular uptake of CpG. The uptake of CpG was 90%±3% and 58%±5% for NPs formulated at w/w ratios of mRNA to CpG of 2:1 and 4:1, respectively (FIG. 17C). This correlates to that the NPs formed with a 4:1 ratio of mRNA to CpG contained a lower dose of FITC-CpG. Moreover, CpG was still present in cells 24 h post-treatment with NPs co-encapsulating mRNA and CpG (FIG. 23C).

In addition to cellular uptake, the NPs need to subsequently facilitate endosomal escape to reach the cytosol for translation of antigen-encoding mRNA. To study endosomal escape, we used NPs encapsulating Cy5-labeled eGFP-encoding mRNA and labeled DC2.4 cells with a LysoTracker dye that stains endosomes/lysosomes to examine co-localization between NPs and endosomes/lysosomes. We used apotome microscopy to visualize cellular uptake of Cy5-mRNA NPs, endosomes/lysosomes, and GFP mRNA transfection 6 h post-treatment (FIG. 17D). We evaluated endosomal escape by quantifying the colocalization of endosomes/lysosomes and Cy5-NPs, with lower colocalization corresponding to effective endosomal escape. The calculated Mander's coefficient of colocalization demonstrated that R18A and R18D NPs facilitated significantly higher endosomal escape than R18C NPs (*P<0.05 and **P<0.01; n=3) (FIG. 17E). Thus, the incorporation of a hydroxyl group (endcapping monomers A and D) instead of an additional amine-group (endcapping monomer C) as endcap-modification promoted endosomal escape. The lower level of endosomal escape for R18C NPs explains why their higher cellular uptake did not result in higher mRNA transfection. Taken together, these results demonstrate that both the polymer hydrophobicity of the side-chains and the chemistry of the endcaps influence NP-mediated intracellular trafficking in DCs and that efficient cellular uptake is necessary, but not sufficient, for efficient mRNA delivery.

### 2.4.4 Targeted Transfection of Splenic DCs In Vivo

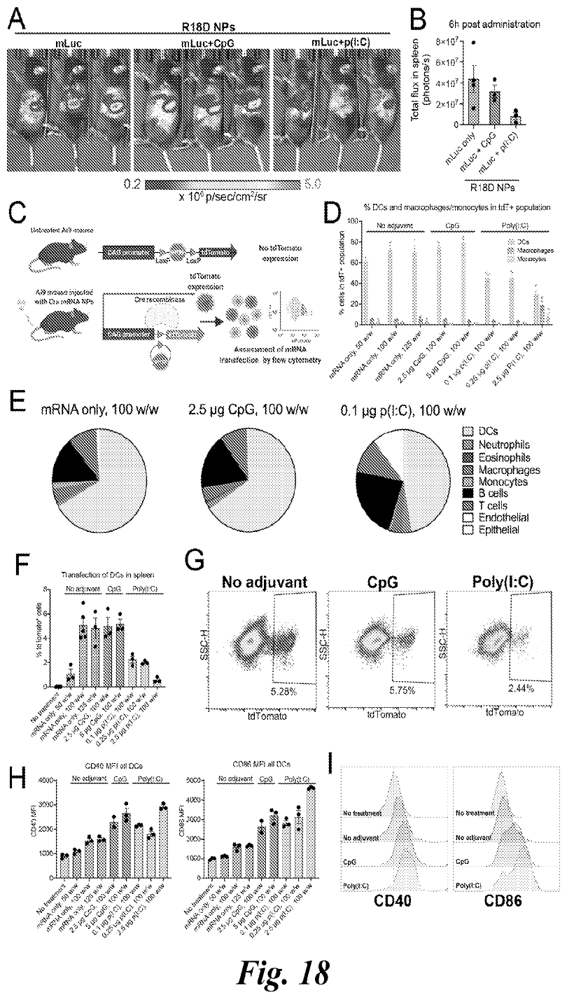

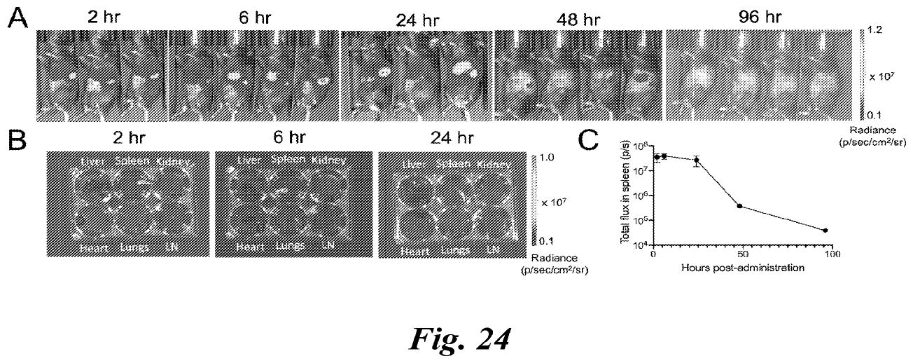

We selected the top performing PBAE NP formulation R18D to move forward into in vivo studies. We first assessed in vivo transfection following systemic administration of fLuc mRNA NPs (10 μg mRNA/mouse) by whole animal bioluminescence imaging over various timepoints post-administration to visualize the localization of the transfection signal and determine duration and peak expression time (FIG. 24). Transfection was localized, almost exclusively, to the spleen (FIG. 24B), and peak expression was between 2 and 6 hours, with continuing expression up to 48 hours post-administration (FIG. 24C). We similarly evaluated in vivo transfection for R18D mRNA-NPs co-delivering no adjuvant, 2.5 μg CpG ODN, or 0.1 μg poly(I:C) 6 hours post-administration (FIG. 18A). Congruent with in vitro DC2.4 transfection results, co-delivery of CpG did not significantly reduce the total bioluminescence in the spleen, while poly(I:C) reduced the bioluminescence signal by approximately 5-fold compared to the no adjuvant group (FIG. 18B). However, all groups, with and without adjuvants, showed localized transfection in the spleen.

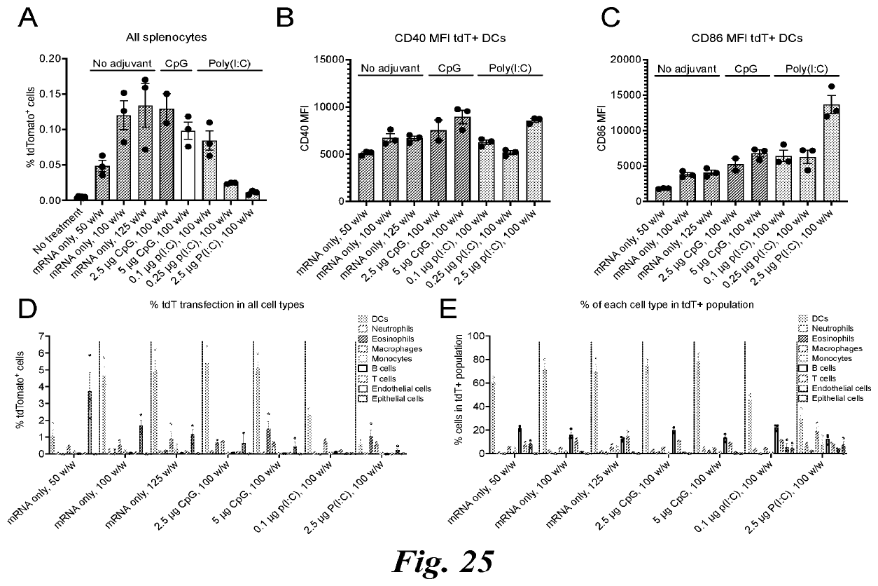

In addition to the organ-level targeting, we were interested in characterizing the specific cell populations in the spleen that are targeted for transfection. We used the Ai9 mouse model in which, upon successful delivery of Cre mRNA, cells undergo Cre recombinase-mediated recombination, resulting in tdTomato expression that can subsequently be detected by flow cytometry (FIG. 18C). Madisen et al., 2010. We assessed transfection in splenic cell populations 24 hours post-treatment with systemically administered R18D NPs encapsulating Cre mRNA alone at varying w/w ratios or co-delivering Cre mRNA with CpG or poly(I:C) at varying adjuvant doses. R18D NPs formed at 50 w/w were less effective than their 100 and 150 w/w counterparts, in terms of both overall splenocyte transfection (FIG. 25A) and transfection of DCs (FIG. 18F). Increasing the polymer to nucleic acid weight ratio from 100 to 150 did not improve NP performance, and, thus, 100 w/w was selected for further in vivo therapeutic studies. R18D NPs facilitated transfection of approximately 5% of DCs in the spleen (FIG. 18F-FIG. 18G, FIG. 25D), and DCs represented the largest share of transfected cells in the spleen, accounting for approximately 70% of all transfected cells, demonstrating cell-specificity of transfection (FIG. 18D-FIG. 18E, FIG. 25E). For R18D NPs co-delivering poly(I:C), DCs represented a smaller share of transfected cells, although still the largest of any cell population, at approximately 45%, with about 2% of splenic DCs transfected for the two lower poly(I:C) doses (FIG. 18D-FIG. 18G, FIG. 25D-FIG. 25E). Poly(I:C) also decreased the overall transfection of splenocytes from approximately 0.13% to 0.08% at the lowest poly(I:C) doses tested (FIG. 25A). Macrophages and monocytes represented a small percentage of the transfected splenocytes, approximately 4-5% and 1-2%, respectively (FIG. 18D-FIG. 18E, FIG. 25E).

In addition to facilitating efficient antigen presentation by DCs, the vaccine formulations must include immunostimulatory components to induce DC activation and elicit a robust immune response. Thus, we also assessed the expression of DC activation markers, CD40 and CD86, in the transgenic Ai9 mouse model. The mRNA NPs on their own led to minimal upregulation of CD40 and CD86, but co-delivery of CpG or poly(I:C) adjuvants led to a substantial increase in expression of those activation markers (FIG. 18H-FIG. 181, FIG. 25B-FIG. 25C). To balance transfection and immunostimulation, the lowest CpG and poly(I:C) doses of 2.5 μg (0.4 nmol) and 0.1 μg per mouse, respectively, were selected for in vivo therapeutic studies. These doses are significantly lower than conventional doses for vaccine adjuvants, which are typically reported to be administered at doses of 10-50 μg/mouse for both CpG, Callmann et al., 2020; Kuai et al., 2017; Liu et al., 2021; Nanishi et al., 2022; Ni et al., 2020; Shih et al., 2021; Wang et al., 2020; Zhang et al., 2021, and poly(I:C). Da Silva et al., 2019; He et al., 2021; Kim et al., 2017; Nakamura et al., 2022; Qin et al., 2021; Roy et al., 2021.

2.4.5 In vivo therapeutic tumor vaccination

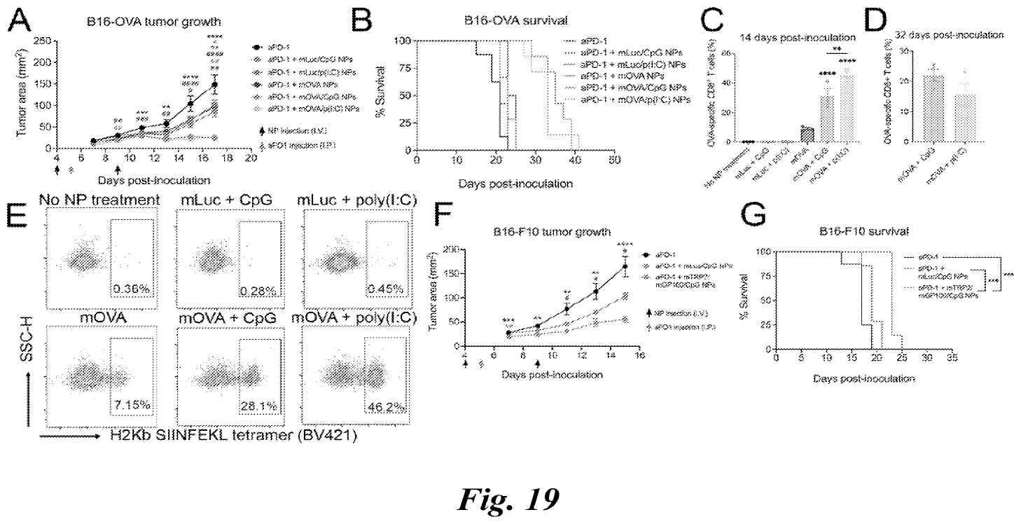

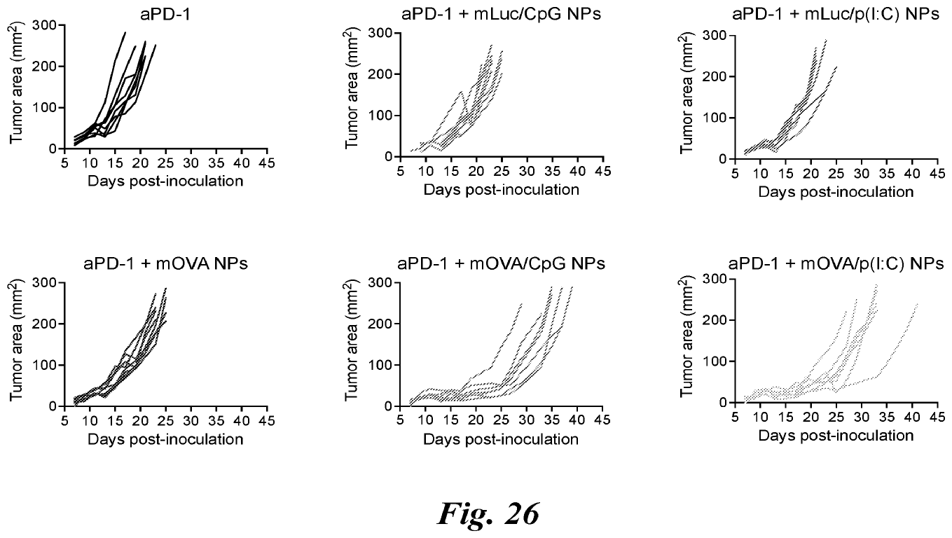

We next examined the therapeutic efficacy of the engineered PBAE mRNA NP vaccination platform in treating in vivo murine tumor models. We first evaluated the vaccine platform in the B16-F10-OVA mouse melanoma model, which expresses the ovalbumin (OVA) antigen, in combination with immune checkpoint blockade. We inoculated C57BL/6J mice (n=7-8 mice/group) subcutaneously on day 0 with B16-F10-OVA cells, followed by intravenous NP administration on days 4 and 9, with systemic anti-PD-1 (aPD-1) treatment on day 5 (FIG. 19A). R18D NPs co-encapsulating OVA mRNA (mOVA) or fLuc mRNA (mLuc, as an irrelevant mRNA control) and CpG, poly(I:C), or no adjuvant were administered. The mOVA/CpG and mOVA/poly(I:C) NP treatments resulted in a statistically significant decrease in tumor burden, completely halting tumor growth for over a week after the final vaccination, while the control treatments had no statistically significant effect on tumor size compared to the aPD-1 only control (FIG. 19A, FIG. 26). The mOVA/CpG and mOVA/poly(I:C) NP treatments also prolonged survival from a median survival of 21 days for the aPD-1 group to 35 and 33 days, respectively (P=0.0002) (FIG. 19B). Additionally, we assessed the presence of OVA-specific CD8+ T cells in the blood 14 days post-inoculation using a tetramer stain for the H-2Kb restricted OVA SIINFEKL epitope (FIG. 19C, FIG. 19E). Controls without OVA mRNA generated almost no antigen-specific CD8+ T cells, while in mice treated with OVA mRNA NPs with no adjuvant, 8.9% of CD8+ T cells in circulation were found to be OVA-specific. The percent of OVA-specific CD8+ T cells in circulation substantially increased with the inclusion of CpG or poly(I:C) adjuvants to 31.0% and 45.1%, respectively, confirming that an immunostimulatory component is required for the NPs to elicit robust antigen-specific T cell proliferation. We assessed OVA-specific T cells again 32 days post-inoculation in surviving mice treated with mOVA/CpG and mOVA/poly(I:C) NPs to analyze the long-term CD8+ T cell response and found that there were still 22.0% and 15.5% OVA-specific CD8+ T cells in the mOVA/CpG and mOVA/poly(I:C) groups, respectively. As expected, the percent of antigen-specific T cells decreased from day 14. Interestingly, despite a somewhat higher CD8+ T cell response in the poly(I:C) group on day 14, although not statistically significant, there was an observed higher percentage of OVA-specific CD8+ T cells in the CpG group on day 32.

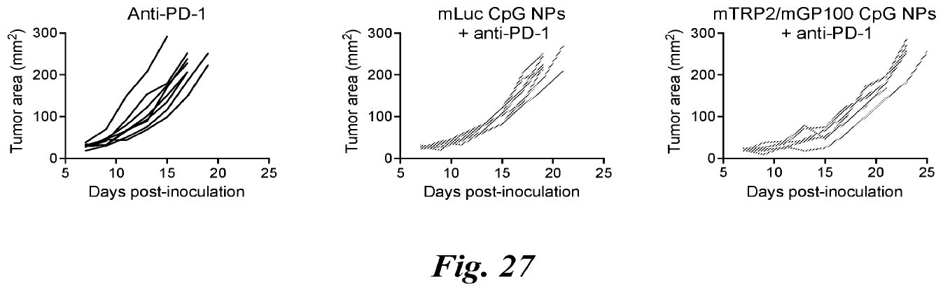

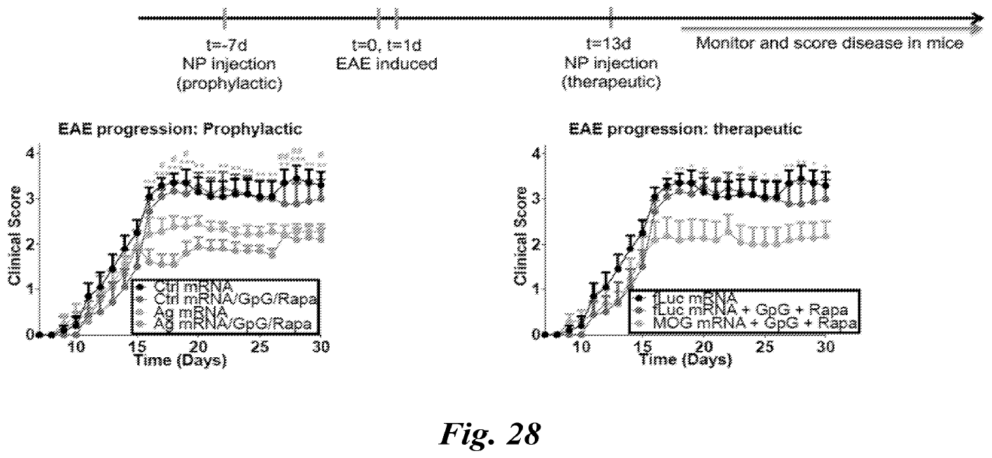

We next assessed the efficacy of the vaccination platform in B16-F10 murine melanoma cells that do not express the immunogenic OVA antigen. We opted to deliver mRNA encoding two well established melanoma-associated antigens, tyrosinase-related protein 2 (TRP2) and glycoprotein 100 (GP100). Kirkin et al., 1998. As TRP2 and GP100 are self-antigens, they are typically difficult to vaccinate against, Pedersen et al., 2013, so we sought to explore whether the NP vaccine platform could be potent enough to exert a therapeutic effect in this model. We inoculated mice with B16-F10 cells and treated them following the same scheme used previously in the B16-F10-OVA model. Following the results from the B16-F10-OVA study in which mOVA/CpG-treated mice had a slightly longer median survival time and a more robust long-term CD8+ T cell response compared to the mOVA/poly(I:C) group, we elected to move forward with the CpG adjuvant for subsequent in vivo tumor studies. Treatment with R18D NPs co-encapsulating a 1:1 weight ratio of TRP2 and GP100 mRNA, in combination with CpG, led to a significant reduction in tumor burden compared to the aPD-1 only control (FIG. 19F, FIG. 27). Median survival was significantly extended (P 0.0001) from 17 days in the aPD-1 control group to 23 days in the full treatment group (FIG. 19G).

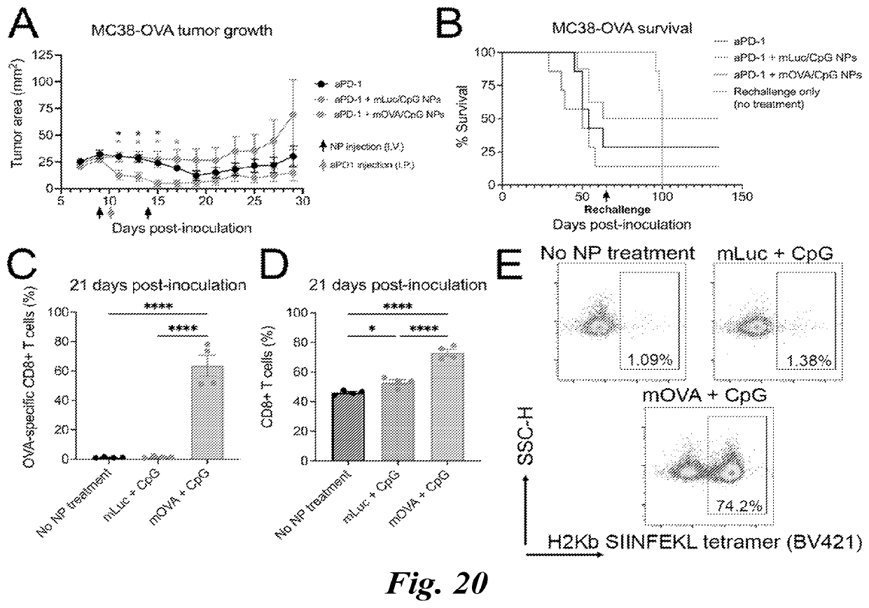

Finally, we investigated the efficacy of the engineered PBAE NP vaccine in the murine colon carcinoma MC38-OVA model to demonstrate utility in treating a different type of tumor. We inoculated C57BL/6J mice (n=7-8 mice/group) subcutaneously with MC38-OVA cells on day 0 and treated with R18D NPs co-encapsulating fLuc- or OVA-encoding mRNA and CpG on days 9 and 14 with aPD-1 administration on day 10 (FIG. 20A). Treatment with mOVA/CpG-NPs significantly reduced tumor burden compared to both controls on days 11-17, and 50% of mice completely cleared their tumors and were long-term survivors compared to only 14.3% and 28.6% in the mLuc/CpG and aPD-1 only groups, respectively (FIG. 20B). On day 65, all long-term survivors, in addition to 7 age-matched mice, were re-challenged with MC38-OVA cells on the opposite flank. All long-term survivors completely rejected the re-challenge. We also assessed OVA-specific CD8+ T cells in the blood 21 days post-inoculation (FIG. 20C-FIG. 20E). There was a dramatic antigen-specific CD8+ T cell response, with 64.1% of CD8+ T cells in the blood being OVA-specific in mOVA/CpG NP-treated mice, compared to approximately 1% in the controls (FIG. 20C, FIG. 20E). Additionally, mOVA/CpG NP-treated mice had a higher percentage of CD8+ T cells out of all CD3+ T cells in the blood compared to both controls (FIG. 20D).

### 2.5 Discussion

Personalized cancer vaccines are an appealing therapeutic approach in which the patient's immune system is activated to trigger antitumor immunity in an antigen-specific manner with the potential for immunologic memory. Cancer vaccines can also be a promising companion therapy to FDA-approved immune checkpoint blockade therapies, as checkpoint blockade is largely ineffective on its own in so-called “cold” tumors that have a low degree of pre-existing T-cell infiltration and may benefit from a vaccine that turns the tumor “hot” by priming a T-cell response. Galon and Bruni, 2019. Additionally, immune checkpoint blockade can aid cancer vaccines in overcoming the immunosuppressive tumor microenvironment and reversing T-cell exhaustion. Saxena et al., 2021. However, clinical translation of cancer vaccines has been challenging due, in part, to insufficient immune responses and antigenic heterogeneity within tumors. Blass and Ott, 2021; Kozielski et al., 2014. Nucleic acid-based vaccines represent a new exciting biotechnology, with mRNA vaccines emerging as a validated platform following the recent FDA approval of Pfizer/BioNTech and Modema's LNP-based vaccines for SARS-CoV-2. Polack et al., 2020; Baden et al., 2021; Shin et al., 2020. mRNA vaccines possess advantages over DNA vaccines, as the cargo only needs to be delivered to the cytosol rather than the nucleus, and there is no risk of insertional mutagenesis. mRNA encoding multiple tumor antigens can be delivered, increasing the chance of therapeutic efficacy. Blass and Ott, 2021; Dagogo-Jack and Shaw, 2018. Additionally, full-length proteins can be encoded, unlike peptide-based vaccines, allowing the presentation of a broader set of epitopes. Finally, mRNA vaccines can result in endogenous presentation by MHC class I, which is critical for generating a cytotoxic CD8+ T-cell response, without the need to rely on inefficient cross-priming. However, advanced mRNA nanocarriers are needed to enable safe and efficient intracellular delivery of both mRNA and adjuvants to DCs following systemic administration.

We synthesized a library of novel bioreducible lipophilic PBAE structures to facilitate fundamental understanding of structure-function relationships and identify candidates for efficient intracellular delivery to DCs. Chemical structures were included to enhance multiple steps of intracellular delivery including cellular uptake, endosomal escape, mRNA release and biodegrading, and, ultimately, transfection. The length of the lipophilic alkyl side-chain was varied to evaluate the effect of backbone lipophilicity, and the polymer endcapping groups were varied to explore the role of differential chemical structure at the terminals of the biomacromolecule. We determined that inclusion of a lipophilic subunit in the PBAE backbone is critical for DC transfection, as PBAE NPs with only the hydrophilic subunit S4 resulted in very minimal cellular uptake and mRNA transfection in vitro. Interestingly, this finding is different from what has been reported for delivery of plasmid DNA, where amino alcohol side chains work well for DNA transfection of many cell types without a lipophilic component. Mangraviti et al., 2015; Shen et al., 2020; Shmueli et al., 2012.

The benefit of the lipophilic subunit in the polymer backbone may be due, in part, to improved mRNA encapsulation and stability of lipophilic PBAE NPs, presumably due to hydrophobic interactions. Additionally, the length of the lipophilic subunit strongly affects cellular uptake and transfection. Polymers with increased lipophilicity showed greater cellular uptake and improved mRNA transfection. In addition to the lipophilic subunit, we found that the chemistry of the endcap molecule influences uptake and subsequent endosomal escape. Within the R18 polymer series (most hydrophobic), R18C NPs facilitated the highest uptake. However, R18A and R18D NPs provided higher endosomal escape compared to R18C, suggesting that the hydroxyl group in endcap monomers A and D is preferable to the additional amine group in endcap monomer C for promoting endosomal escape. Balancing uptake and endosomal escape, R18D NPs facilitated the highest in vitro DC transfection among all structures in the library. Additionally, R18D NPs transfected DC2.4 cells and murine BMDCs at significantly higher levels in vitro compared to leading commercial mRNA transfection reagents, even at very low mRNA doses.

We examined the tissue- and cell-level targeting of R18D NPs following systemic administration. R18D NPs were found to transfect the spleen with a high degree of specificity, largely avoiding the liver, which is usually a major site of NP accumulation and transfection for many lipid- and polymer-based nanocarriers. Zhang et al., 2016; Samaridou et al., 2020. Furthermore, within the spleen, DCs were preferentially transfected over other cell types, including macrophages. This is in contrast to most non-viral gene delivery platforms, in which macrophages and monocytes rapidly phagocytose NPs upon systemic administration and represent a large proportion of transfected cells, Wilhelm et al., 2016, and even LNPs that can transfect non-macrophage populations largely target hepatocytes. Samaridou et al., 2020. Previous work has generally required surface functionalization with DC-targeting ligands to achieve such specificity. Tacken et al., 2007; Phua et al., 2015. For a cancer vaccine application, delivery to DCs over other APCs, namely monocytes and macrophages, is preferred as DCs are much more potent in initiating a T cell response. Pozzi et al., 2005. We also demonstrated the ability of the engineered polymeric NPs to co-encapsulate and deliver the nucleic acid-based TLR agonists CpG and poly(I:C) along with mRNA. By co-delivering mRNA and adjuvant within the same PBAE NPs, we ensure that any DC that is transfected with antigen-encoding mRNA also receives a danger signal, resulting in its activation. R18D mRNA-NPs co-delivering CpG or poly(I:C) were able to activate splenic DCs at very low adjuvant doses, as low as 2.5 μg CpG or 0.1 μg poly(I:C) per mouse. These doses are substantially lower than previously reported doses, which are typically in the range of 10-50 μg/mouse for both CpG, Callmann et al., 2020; Kuai et al., 2017; Liu et al., 2021; Nanishi et al., 2022; Ni et al., 2020; Shih et al., 2021; Wang et al., 2020; Zhang et al., 2021, and poly(I:C). Da Silva et al., 2019; He et al., 2021; Kim et al., 2017; Nakamura et al., 2022; Qin et al., 2021; Roy et al., 2021.

The engineered NPs facilitated highly targeted delivery to splenic DCs with efficient cellular internalization. As a result, the reported NP design enables a therapeutic effect with much lower adjuvant doses and avoids delivery to off-target cells, potentially reducing the risk of systemic side effects. The simplicity of the non-viral nanoparticles (consisting of a single defined polymer, rather than requiring a complex mixture of lipids, PEG, and cholesterol) as well as the ease with which the nanoparticles are formulated (simple mixing of the polymer with any combination of antigen-encoding mRNAs and nucleic acid-based adjuvants in aqueous conditions) also enables manufacture, translation, and ultimately accessibility of this biotechnology.

Finally, we demonstrated therapeutic efficacy of the engineered PBAE NP vaccine platform in multiple in vivo tumor models in combination with anti-PD-1 immune checkpoint blockade. R18D NPs co-delivering antigen mRNA and CpG or poly(I:C) showed efficacy in eliciting an antitumor response in B16-F10-OVA and B16-F10 murine melanoma models and MC38-OVA murine colon carcinoma. In the B16-F10-OVA and MC38-OVA tumor models, we observed a dramatic antigen-specific CD8+ T cell response following treatment with NPs co-delivering antigen mRNA and adjuvant, resulting in reduced tumor burdens and prolonged median survival. NPs without CpG or poly(I:C) did not potentiate a strong antigen-specific T cell response or antitumor therapeutic efficacy, which confirmed that an immunostimulatory adjuvant is required for this cancer vaccine platform to elicit a robust immune response. Additionally, NPs delivering fLuc mRNA as an irrelevant 10 mRNA control, along with CpG or poly(I:C), did not mediate a significant antitumor response. This validates that the effect mediated by the vaccination platform is not solely due to adjuvant immunogenicity and is, in fact, an antigen-specific response. In the MC38-OVA model, 50% of mice treated with OVA mRNA and CpG completely cleared their tumors and survived a re-challenge approximately 50 days after the final NP treatment, suggesting that this platform induces long-term systemic immunity. We also assessed the efficacy of the R18D NP platform in treating B16-F10 by targeting TRP2 and GP100, which are well established melanoma self-antigens. Kirkin et al., 1998. Tumor-associated self-antigens are typically very difficult to vaccinate against due to immune tolerance, Pedersen et al., 2013, but the PBAE NP vaccine platform was able to overcome tolerance to mediate a significant reduction in tumor size and prolonged survival in this model. Overall, the PBAE NP-mRNA vaccine showed efficacy in treating three different in vivo tumor models by incorporating low adjuvant doses and targeting clinically relevant antigens, demonstrating the translational promise of this platform and potential for application to neoantigen vaccines. Compared to virus-based strategies or ex vivo engineered cell-based immuno-oncology strategies, the biodegradable polymeric nanoparticle vaccine platform discussed here overcomes many safety, manufacturing, and scalability challenges. Compared to traditional and emerging lipid nanoparticle technology, safe, efficient, and specific combination mRNA delivery to splenic dendritic cells in vivo was achieved without the need for PEGylation or targeting ligands, without most of the delivery being focused on the liver, and with a wide therapeutic window.

### 2.6 Summary

In summary, we successfully engineered novel bioreducible lipophilic PBAE NPs with enhanced properties for safe and efficient mRNA delivery to DCs. Utilizing a combinatorial library screening approach, we identified key structural components of PBAE nanocarriers for efficient mRNA transfection of DCs in vitro by exploring structure-function relationships that influence cellular uptake and endosomal escape. The PBAE nanoparticles are similarly able to transfect DCs at high levels in vivo, including while flexibly co-encapsulating nucleic acid-based adjuvants. Following systemic delivery, engineered PBAE NPs specifically target the spleen, the major site of immune cells, while avoiding sequestration in the liver, the typical site of NP accumulation. Within the spleen, the PBAE NPs selectively transfect DCs over other cell populations, including monocytes and macrophages, at high levels. Finally, we demonstrated therapeutic efficacy of the engineered NP platform in reducing tumor burden and extending survival in B16-F10-OVA, B16-F10, and MC38-OVA models, demonstrating the capability to vaccinate against self-antigens and induce tumor regression and long-term survival. These three models demonstrate the versatility and potential clinical utility of this platform. This biotechnology platform is a simple, modular, and scalable method for in vivo production of cancer antigen-specific CD8+T cells that sidesteps the many challenges of alternative viral and/or ex vivo cellular engineering strategies. Taken together, these results show tremendous promise for the use of bioreducible lipophilic PBAE NPs as a modular genetic vaccine.

### 2.7 Methods

2.7.1 Monomer and polymer synthesis

The bioreducible monomer 2,2′-disulfanedivlbis(ethane-2,1-diyl) diacrylate (R) was synthesized as previously described. Kozielski et al., 2014; Karlsson et al., 2021a. In brief, 2-hydroxyethyl disulfide (Sigma-Aldrich) was acrylated in dichloromethane (DCM) with acryloyl chloride as the acrylation reagent and in presence of triethylarnine (TEA) (Sigma-Aldrich) overnight at room temperature. The TEA HC precipitate was removed by filtration and the product washed with water and dried with sodium sulfate, and the solvent was removed by rotary evaporation. Bioreducible lipophilic PBAEs were synthesized via Michael addition reaction. In the first step, the diacrylate backbone monomer R and a combination of the hydrophilic side chain amine-containing monomer 4-amino-1-butanol (S4) and a lipophilic amine-containing side chain monomer (1-dodecylamine [Sc12], tetradecylarnine [Sc14], hexadecylamine ISc16I, or oleylarnine [Sc18]) were dissolved in anhydrous dimethvlfonra.mide (DMF), and the reaction proceeded for 24 h at 85° C. with stirring. The diacrylate monomer to total amine monomer molar ratio was 1.05:1 at a total monomer concentration of 500 mg/mL, and the hydrophilic to lipophilic side chain molar ratio was 1:1 unless otherwise noted. In the synthesis of bioreducible PBAEs without a lipophilic subunit only S4 was used as side chain monomer. In the second step, the obtained acrylate-terminated polymer was dissolved together with an endcapping monomer (2-(3-aminopropylamino)ethanol [A], N,N-Bis(2-hydroxyethyl)ethylenediamine [B], Diethylentriamine [C], N-(2-Hydroxyethyl)ethylenediamine [D], or N-(3-Aminopropyl)piperidine [E]) (0.5 M endcap monomer and 200 mg/mL of base polymer) in tetrahvdrofuran (TIHF), and the reaction proceeded for 1 hour at room temperature to form the final polymer. The obtained polymers were purified by precipitation in a 1:1 mixture of diethyl ether and hexane and two washes. The polymers were dried under vacuum for 48 hours and then dissolved in anhydrous dimethyl sulfoxide (DMSO) at 100 mg/mL and stored as aliquots at −80° C. with desiccant.

### 2.7.2 NP Preparation

NPs were formed by first separately dissolving polymer and nucleic acid cargo, including mRNA. CpG oligodeoxynucleotide (ODN 1826; Invivogen), and/or poly(I:C) high molecular weight (HMW) (Invivogen), in 25 mM sodium acetate buffer (NaAc, pH 5) at specified concentrations. The polymer and nucleic acid cargo were then mixed together at specified w/w ratio (100-300 w/w) and allowed to self-assemble into NPs for 6 min at room temperature.

### 2.7.3 Polymer and NP Characterization

Polymer molecular weight was measured using gel permeation chromatography (GPC) relative to linear polystyrene standards using a refractive index detector (Waters). Prior to measurements, polymers were dissolved in butylated hydroxytoluene (BI-T)-stabilized TIHF and filtered through 0.2-μm polytetrafhuoroethylene (PTFE) filters. The hydrodynamic diameter of the NPs in 1×PBS was measured by dynamic light scattering (DLS) using a Zetasizer Pro (Malvem Panalytical). Zeta potential was measured via electrophoretic mobility using same instrument to characterize the surface charge of the NPs. NP size and morphology was visualized by TEM using a Talos 1120C microscope (Thermo Scientific). NP samples of 20 μL were added to carbon-coated copper TEM grids (Electron Microscopy Sciences) for 10 min. The grids were then washed three times for 10 seconds each with MilliQ water and blotted in between and after and allowed to dry at room temperature for 20 min before imaging.

2.7.4 Cell culture and cell line preparation

Murine DC2.4 cells were cultured in RPMI 1640 media (Gibco) supplemented with 10% fetal bovine serum (FBS), 1% penicillin/streptomycin, 10 nM HEPES, 1X non-essential amino acids, and 50 μM beta-mercaptoethanol. Bone marrow-derived dendritic cells (BMDCs) were generated from bone marrow isolated from C57BL/6J mice (Jackson Laboratory: Bar Harbor, ME). On day 0, bone marrow was flushed from femurs and tibias of mice, filtered through 70-pam sterile nylon mesh and then resuspended in 5 mL ACK lysis buffer. Cells were plated in a 6-well plate at 1×10⁶ cells/mL in RPMI 1640 media supplemented with 10% FBS, 1% penicillin/streptomycin, 50.M beta-mercaptoethanol, and 20 ng/nmL recombinant murine G1M-CSF (Peprotech; Cranbury, NJ). On day 3, an equal volume of media with 40 ng/mL GM-CSF was added. Cells were harvested on day 6 by collecting loosely adhered cells. B16-F10 and B16-F10-OVA cells were cultured in DMEM high glucose with sodium pyruvate (Gibco) supplemented with 10% FBS and 1% penicillin/streptomycin, with the addition of 5 mg/mL G418 for B16-F10-OVA cells. MC38-OVA cells were cultured in RPMI 1640 supplemented with 10% FBS and 1% penicillin/streptomycin.

2.7.5 In vitro mRA 4 transfection

Unless stated otherwvise, all mRNA used was purchased from TriLink Biotechnologies with 5-Methoxyuridine modification and CleanCap technology. Cells were plated at 15,000 cells per well in 100 μL of medium in 96-well plates (unless stated otherwise) and allowed to adhere overnight. NPs were formulated as described above with eGFP mRNA (and CpG or Poly(I:C) if indicated) and then added to the cells. After a 2 hour incubation at 3⁷° C., medium with NPs was replaced with 100 pdL. of fresh medium. Lipofectamine MessengerMAX (Thermo Fisher Scientific) and jetMESSENGER. (Polyplus) were used for comparison to commercial mRNA transfection reagents following manufacturer protocols. For cellular uptake studies, 20% of the total mRNA was replaced with Cy5-labeled eGFP mRNA (Trilink Biotechnologies). After 6 hours (for uptake experiments) or 24 hours (for transfection experiments), transfection/uptake was evaluated via flow cytometry using an Attune NxT Flow Cytometer (Thermo Fisher). The MTS CellTiter 96 Aqueous One (Promega) cell proliferation assay was perforned 24 hours post-transfection according to the manufacturer's instructions as a measure of cell viability. The metabolic activity of treated cells was normalized to that of untreated cells.

2.7.6 Encapsulation efficiency and NP stability

mRNA encapsulation efficiency was assessed using the RiboGreen RNA assay (Invitrogen) following manufacturer's protocols. In brief, NPs were added to a 96-black-well plate and incubated in either PBS or 10 mg/mL heparin solution. Ribogreen reagent was added and fluorescence readings were performed using a Biotek Synergy 2 fluorescence multiplate reader (BioTek) to compare encapsulation efficiency for NPs in PBS compared to free mRNA (NPs in presence of heparin). The gel electrophoresis assay was performed to examine NP stability when incubated in PBS or 10% serum for 4 h. NPs were formed with Cv5-labeled mRNA (Tni Link Biotechnologies) alone or Cy5-labeled mRNA and FITC-labeled CpG ODN (ODN 1826; InvivoGen). Nucleic acid cargos were incubated alone as controls for calculation of % dissociation of the NP cargo. Samples were loaded in an 1% agarose (UltraPure Agarose, Invitrogen) gel, and the gel was run for 20 mn at 100 V and imaged with iBright FL1500 Imaging System (Thermo Fisher).

### 2.7.7 Characterization of Intracellular Trafficking

Endosomal escape of NPs was studied in DC2.4 cells using immunofluorescence staining. Cells were plated onto coverslips in 12-well plates and grown overnight. NPs were prepared with 20% Cy5-mRNA and 80% unlabeled mRNA and incubated with cells for 6 h. Cells were washed with PBS and then stained for 30 min with Hoechst 33342 (Thermo Fisher Scientific) nuclear stain at 1:5000 dilution and Cell Navigator Lysosome Staining dye (AAT Bioquest) at 1:2500 dilution in complete media. Stained cells were washed twice in PBS and then fixated in 10% formalin. Fixated cells were washed twice in PBS and once in MilliQ H₂O and mounted on slides with SlowFade Diamond antifade mounting medium (Life Technologies) and sealed. A Zeiss Z2 Axio Observer with Apotome microscope (Zeiss) and a 63× oil immersion lens was used to visualize transfection, cellular uptake, and endosomal escape of NPs. Colocalization of Cy5-NPs and lysotracker stain was quantified by calculating the Manders' coefficient in acquired images using ImageJ.

### 2.7.8 NP Formulation for In Vivo Transfection

All animal work was performed in strict adherence of the policies and guidelines of the Johns Hopkins University Animal Care and Use Committ^{e}e (ACUC). NPs for in vivo mRNA delivery were formulated at 100 w/w unless stated otherwise. All in vivo transfections utilized the R18D polymer (BR6-S4, Sc18 [50:50]-D). R18D and mRNA/adjuvants were diluted separately in NaAc and then mixed at a 4:1 volume ratio. VacciGrade poly(I:C) (1MW) and CpG ODN 1826 were used for in vivo studies (Invivogen). NPs were allowed to assemble at room temp for 6 minutes, and then a 500 mg/mL sucrose solution was used to bring the solution to isotonicity. NPs were administered to animals via 200 μL intravenous injections (by tail vein injection for Ai9 studies or by retro-orbital injection for luciferase studies and tumor studies).

2.7.9 In vivo bioluminescence transfection studies

NPs encapsulating fLuc mRNA were formulated as described above and administered to 6- to 7-week-old male C57BL/6J mice via retro-orbital injection. Mice were shaved and whole-body bioluminescence was assessed at 6 hours post-injection (or at prespecified timepoints up to 96 hours for time course study). D-Luciferin potassium salt solution (25 mg/mL in PBS; Cayman Chemical Company) was administered to mice via. 150 μL intraperitoneal injections, and mice were imaged using the IVIS Spectrum Imager (Perkin Elmer) after 10 min. For timecourse studies, animals were euthanized immediately after whole-body imaging via cervical dislocation, and selected organs were extracted, submerged in D-luciferin solution (250 μg/mL), and imaged with IVIS.

2.7.10 Cre mRNA delivery to Ai9 mice

Ai9 mice were purchased from Jackson Laboratory (JAX stock #007909) and bred in the Johns Hopkins animal facility. Madisen et al., 2010. NPs encapsulating Cre mRNA and adjuvants (if indicated) were administered to Ai9 mice via tail vein injections, and tdTomato expression following Cre-Lox recombination was allowed to accumulate for 24 hours, at which point animals were euthanized via cervical dislocation. Spleens were extracted and dissociated by a 1-hour incubation in collagenase D (2 mg/mL) at 37° C., followed by mechanical pressing through a 70-μm cell strainer. Cells were pelleted by centrifugation, the supernatant was removed, and red blood cells in the cell pellet were lysed by incubating in ACK lysis buffer (Quality Biological) for 1 min at room temperature. Cells were diluted in PBS, passed through a 100-μm cell strainer, pelleted by centrifugation, and resuspended in fluorescence-activated cell sorting (FACS) buffer (2% FBS in PBS with 0.02% sodium azide). Surface staining of cells with fluorescent antibodies was then performed using the antibodies and dilutions listed in Table 1 in FACS buffer for 30 min at 4° C., at which time cells were washed twice and resuspended in FACS buffer for further analysis. Flow experiments were performed using an Attune NxT flow cytometer and analyzed using FlowJo software (FlowJo).

TABLE 1
Antibodies used for Ai9 flow cytometry studies.

| | | | | Catalog |
|---|---|---|---|---|
| Antibody | Dilution | Clone | Supplier | No. |
| BV421 anti-CD45 | 1:100 | 30-F11 | Biolegend | 103134 |
| BV605 anti-CD86 | 1:20 | GL-1 | Biolegend | 105037 |
| AlexaFluor 488 anti-CD11b | 1:100 | M1/70 | Biolegend | 101217 |
| PerCP anti-CD40 | 1:20 | 3/23 | Biolegend | 124624 |
| APC anti-CD11c | 1:100 | N418 | Biolegend | 117310 |
| AlexaFluor 700 anti-I-A/I-E | 1:100 | M5/114.14.2 | Biolegend | 107622 |
| APC/Cy7 anti-Ly6G | 1:100 | 1A8 | Biolegend | 127624 |
| AlexaFluor 488 anti-CD3 | 1:80 | 17A2 | Biolegend | 100210 |
| APC anti-CD19 | 1:100 | 6D5 | Biolegend | 115512 |
| AlexaFluor 700 anti-CD31 | 1:100 | 390 | Biolegend | 102444 |
| APC/Cy7 anti-CD326 | 1:80 | G8.8 | Biolegend | 118218 |

### 2.7.11 Tumor Vaccination Studies

For both B16-F10-OVA and B16-F10 tumor studies, 3×10⁵ cells in 50 μL RPMI 1640 media were inoculated subcutaneously in the right flank of C57BL/6J mice on day 0. For MC38-OVA studies, 1×10⁶ cells in 50 μL RPMI 1640 media were inoculated subcutaneously in the right flank of C57BL/6J mice on day 0. R18D NPs encapsulating fLuc mRNA (as an irrelevant mRNA control) or OVA mRNA were administered intravenously by retro-orbital injections on days 4 and 9 for the B16-F10-OVA study or days 9 and 14 for the MC38-OVA study at 10 μg mRNA/mouse and 2.5 μg CpG or 0.1 μg poly(I:C) for adjuvant groups (n=7-8 mice/group). For the B16-F10 study, instead of OVA mRNA, a 1:1 mixture (each at 5 μg/mouse) of custom-synthesized TRP2 mRNA (NCBI gene accession number: NM_021882) and GP100 mRNA (NCBI gene accession number: NM 010024) (TriLink Biotechnologies) was used, and NPs were administered intravenously by retro-orbital injection on days 4 and 9. 200 μg of aPD-1 was injected intraperitoneally on day 5 for B116 studies or day 10 for the MC38-OVA study. For all studies, tumor area was measured by caliper every other day starting on day 7 by a blinded researcher, and mice were euthanized by cervical dislocation once the tumor reached 200 mm². For the MC38-OVA study, on day 65, mice that had completely cleared their tumors (n=4 mice for mOVA/CpG NP group, n 2 mice for aPD-1 group, and n=1 mouse for mLuc/CpG group) and 7 age-matched mice were rechallenged with subcutaneous inoculation of 1×10⁶ MC38-OVA cells on the left flank. For B16-F10-OVA and MC38-OVA studies, approximately 100 μL of blood was collected from mice via cheek bleeds and assessed for the presence of OVA-specific CD8+ T cells at the time points stated. Blood samples were resuspended in ACK lysis buffer, washed in PBS and resuspended in FACS buffer with TruStain FeX antibody (BioLegend). Cells were then stained for 45 minutes at 4° C. with AlexaFluor 488 anti-CD3 (Table 1), APC anti-CD8a at 1:80 dilution (BioLegend, catalog no. 100712, clone 53-6.7), PerCP anti-CD4 at 1:80 dilution (BioLegend, catalog no. 100431, clone GK1.5), and BV421 H-2Kb SIINFEKI tetramer or BV421 H-2Kb SSIEFARL tetramer (NIH Tetramer Core Facility) as a negative control at 1:100 dilutions. Samples were analyzed with the Attune NxT flow cytometer.

### 2.7.12 StatisticalAnalysis

All experiments were performed with n=4 replicates unless otherwise stated. Bar graphs indicate mean±standard error of the mean. n.s. (not significant) indicates P>0.05, * indicates P<0.05, ** indicates P<0.01, *** indicates P<0.001, and **** indicates P<0.0001. For studies with two variables, two-way ANOVA was used with recommended post-hoc tests. For studies with only one variable, one-way ANOVA was used with recommended post-hoc tests. For tumor studies, one-way ANOVA with post-hoc Tukey's test was used to compare tumor sizes on each day, and a log rank (Mantel-Cox) test was uses to assess significance between survival curves. All statistics were performed using statistical analysis software modules in GraphPad Prism 9 (GraphPad Software, Inc.) and P<0.05 was considered statistically significant.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Blass, E. and Ott, P. A. Advances in the development of personalized neoantigen-based therapeutic cancer vaccines. Nature Reviews Clinical Oncology 18, 215-229 (2021).
Whiteside, T. L., Demaria, S., Rodriguez-Ruiz, M. E., Zarour, H. M. and Melero, I. Emerging opportunities and challenges in cancer immunotherapy. Clinical Cancer Research 22, 1845-1855 (2016).
Vormehr, M., Tureci, O. and Sahin, U. Harnessing tumor mutations for truly individualized cancer vaccines. Annual Review of Medicine 70, 395-407 (2019).
Wang, R. F. and Wang, H. Y. Immune targets and neoantigens for cancer immunotherapy and precision medicine. Cell Research 27, 11-37 (2017).
Hu, Z., Ott, P. A. and Wu, C. J. Towards personalized, tumour-specific, therapeutic vaccines for cancer. Nature Reviews Immunology 18, 168-182 (2018).
Pardi, N., Hogan, M. J., Porter, F. W. and Weissman, D. mRNA vaccines-a new era in vaccinology. Nature Reviews Drug Discovery 17, 261-279 (2018).
Sahin, U., Karikó, K. and Tureci, O. MRNA-based therapeutics-developing a new class of drugs. Nature Reviews Drug Discovery 13, 759-780 (2014).
Kaczmarek, J. C., Kowalski, P. S. and Anderson, D. G. Advances in the delivery of RNA therapeutics: From concept to clinical reality. Genome Medicine 9, 1-16 (2017).
Polack, F. P. et al. Safety and Efficacy of the BNT162b2 mRNA Covid-19 Vaccine. New England Journal of Medicine 383, 2603-2615 (2020).
Baden, L. R. et al. Efficacy and Safety of the mRNA-1273 SARS-CoV-2 Vaccine. New England Journal of Medicine 384, 403-416 (2021).
Shin, M. D. et al. COVID-19 vaccine development and a potential nanomaterial path forward. Nature Nanotechnology 15, 646-655 (2020).
Kim, J., Eygeris, Y., Gupta, M. and Sahay, G. Self-assembled mRNA vaccines. Advanced Drug Delivery Reviews 170, 83-112 (2021).
Adams, D. et al. Patisiran, an RNAi Therapeutic, for Hereditary Transthyretin Amyloidosis. New England Journal of Medicine 379, 11-21 (2018).
Akinc, A. et al. The Onpattro story and the clinical translation of 15 nanomedicines containing nucleic acid-based drugs. Nature Nanotechnology 14, 1084-1087 (2019).
Karlsson, J., Vaughan, H. J. and Green, J. J. Biodegradable polymeric nanoparticles for therapeutic cancer treatments. Annual Review of Chemical and Biomolecular Engineering 9, 105-127 (2018).
Lopez-Bertoni, H. et al. Bioreducible Polymeric Nanoparticles Containing Multiplexed Cancer Stem Cell Regulating miRNAs Inhibit Glioblastoma Growth and Prolong Survival. Nano Lett 18, 4086-4094 (2018).
Karlsson, J. et al. Engineered nanoparticles for systemic siRNA delivery to malignant brain tumours. Nanoscale 11, 20045-20057 (2019a).
Rui, Y. et al. High-throughput and high-content bioassay enables tuning of polyester nanoparticles for cellular uptake, endosomal escape, and systemic in vivo delivery of mRNA. Sci Adv 8, eabk2855 (2022).
Sunshine, J. C., Peng, D. Y. and Green, J. J. Uptake and transfection with polymeric nanoparticles are dependent on polymer end-group structure, but largely independent of nanoparticle physical and chemical properties. Mol Pharm 9, 3375-3383 (2012).
Karlsson, J., Rhodes, K. R., Green, J. J. and Tzeng, S. Y. Poly(beta-amino ester)s as gene delivery vehicles: challenges and opportunities. Expert Opinion on Drug Delivery (2020).
Kozielski, K. L., Tzeng, S. Y., Hurtado De Mendoza, B. A. and Green, J. J. Bioreducible Cationic Polymer-Based Nanoparticles for Efficient and Environmentally Triggered Cytoplasmic siRNA Delivery to Primary Human Brain Cancer Cells. ACS Nano 8, 3232-3241 (2014).
Zamboni, C. G. et al. Polymeric nanoparticles as cancer-specific DNA delivery vectors to human hepatocellular carcinoma. Journal of Controlled Release 263, 18-28 (2017).
Sunshine, J. et al. Small-molecule end-groups of linear polymer determine cell-type gene-delivery efficacy. Advanced Materials 21, 4947-4951 (2009).
Tzeng, S. Y. et al. Non-viral gene delivery nanoparticles based on Poly(p-amino esters) for treatment of glioblastoma. Biomaterials 32, 5402-5410 (2011).
Patel, A. K. et al. Inhaled Nanoformulated mRNA Polyplexes for Protein Production in Lung Epithelium. Advanced Materials 31 (2019).
Iwasaki, A. and Medzhitov, R. Toll-like receptor control of the adaptive immune responses. Nature Immunology 5, 987-995 (2004).
Maisonneuve, C., Bertholet, S., Philpott, D. J. and De Gregorio, E. Unleashing the potential of NOD- and toll-like agonists as vaccine adjuvants. Proceedings of the National Academy of Sciences of the United States of America 111, 12294-12299 (2014).
Gale, E. C. et al. A Nanoparticle Platform for Improved Potency, Stability, and Adjuvanticity of Poly(I:C). Advanced Therapeutics 3, 1900174-1900174 (2020).
Roth, G. A. et al. Designing spatial and temporal control of vaccine responses. Nature Reviews Materials 7, 174-195 (2021).
Karikó, K. et al. Incorporation of pseudouridine into mRNA yields superior nonimmunogenic vector with increased translational capacity and biological stability. Molecular Therapy 16, 1833-1840 (2008).
Pastor, F. et al. An RNA toolbox for cancer immunotherapy. Nature Reviews Drug Discovery 17, 751-767 (2018).
Verdegaal, E. M. E. et al. Neoantigen landscape dynamics during human melanoma-T cell interactions. Nature 536, 91-95 (2016).
Dagogo-Jack, I. and Shaw, A. T. Tumour heterogeneity and resistance to cancer therapies. Nature Reviews Clinical Oncology 15, 81-94 (2018).
Ott, P. A. et al. An immunogenic personal neoantigen vaccine for patients with melanoma. Nature 547, 217-221 (2017).
McLane, L. M., Abdel-Hakeem, M. S. and Wherry, E. J. CD8 T Cell Exhaustion During Chronic Viral Infection and Cancer. Annual Review of Immunology 37, 457-495 (2019).
Meng, F., Hennink, W. E. and Zhong, Z. Reduction-sensitive polymers and bioconjugates for biomedical applications. Biomaterials 30, 2180-2198 (2009).
Manickam, D. S. et al. Effect of innate glutathione levels on activity of redox-responsive gene delivery vectors. Journal of Controlled Release 141, 77-84 (2010).
Karlsson, J. et al. Engineered nanoparticles for systemic siRNA delivery to malignant brain tumours. Nanoscale 11, 20045-20057 (2019b).
Suk, J. S., Xu, Q., Kim, N., Hanes, J. and Ensign, L. M. PEGylation as a strategy for improving nanoparticle-based drug and gene delivery. Advanced Drug Delivery Reviews 99, 28-51 (2016).
Karlsson, J. et al. Photocrosslinked Bioreducible Polymeric Nanoparticles for Enhanced Systemic siRNA Delivery as Cancer Therapy. Advanced Functional Materials, 2009768-2009768 (2021a).
Lopez-Bertoni, H. et al. Bioreducible Polymeric Nanoparticles Containing Multiplexed Cancer Stem Cell Regulating miRNAs Inhibit Glioblastoma Growth and Prolong Survival. Nano Letters 18, 4086-4094 (2018).
Harayama, T. and Riezman, H. Understanding the diversity of membrane lipid composition. Nature Reviews Molecular Cell Biology 19, 281-296 (2018).
Mitchell, M. J. et al. Engineering precision nanoparticles for drug delivery. Nature Reviews Drug Discovery 20, 101-124 (2020).
Blanco, E., Shen, H. and Ferrari, M. Principles of nanoparticle design for overcoming biological barriers to drug delivery. Nature Biotechnology 33, 941-951 (2015).
Wang, Y., Ye, M., Xie, R. and Gong, S. Enhancing the In Vitro and In Vivo Stabilities of Polymeric Nucleic Acid Delivery Nanosystems. Bioconjugate Chemistry 30, 325-337 (2019).
Bessis, N., GarciaCozar, F. J. and Boissier, M. C. Immune responses to gene therapy vectors: influence on vector function and effector mechanisms. Gene Therapy 11, S10-S17 (2004).
Madisen, L. et al. A robust and high-throughput Cre reporting and characterization system for the whole mouse brain. Nature Neuroscience 13, 133-140 (2010).
Callmann, C. E. et al. Tumor cell lysate-loaded immunostimulatory spherical nucleic acids as therapeutics for triple-negative breast cancer. Proceedings of the National Academy of Sciences 117, 17543-17550 (2020).
Kuai, R., Ochyl, L. J., Bahjat, K. S., Schwendeman, A. and Moon, J. J. Designer vaccine nanodiscs for personalized cancer immunotherapy. Nature Materials 16, 489-496 (2017).
Liu, B. et al. Equipping Cancer Cell Membrane Vesicles with Functional DNA as a Targeted Vaccine for Cancer Immunotherapy. Nano Letters 21, 9410-9418 (2021).
Nanishi, E. et al. An aluminum hydroxide:CpG adjuvant enhances protection elicited by a SARS-CoV-2 receptor binding domain vaccine in aged mice. Science Translational Medicine 14, eabj5305 (2022).
Ni, Q. et al. A bi-adjuvant nanovaccine that potentiates immunogenicity of neoantigen for combination immunotherapy of colorectal cancer. Science Advances 6, eaaw6071 (2020).
Shih, T.-Y., Najibi, A. J., Bartlett, A. L., Li, A. W. and Mooney, D. J. Ultrasound-triggered release reveals optimal timing of CpG-ODN delivery from a cryogel cancer vaccine. Biomaterials 279, 121240 (2021).
Wang, H. et al. Biomaterial-based scaffold for in situ chemo-immunotherapy to treat poorly immunogenic tumors. Nature Communications 11, 5696 (2020).
Zhang, Y. et al. Supramolecular Assembled Programmable Nanomedicine As In Situ Cancer Vaccine for Cancer Immunotherapy. Advanced Materials 33, 2007293 (2021).
Da Silva, C. G. et al. Co-delivery of immunomodulators in biodegradable nanoparticles improves therapeutic efficacy of cancer vaccines. Biomaterials 220, 119417 (2019).
He, X. et al. HPV-Associated Tumor Eradication by Vaccination with Synthetic Short Peptides and Particle-Forming Liposomes. Small 17, 2007165 (2021).
Kim, S.-Y. et al. Synthetic vaccine nanoparticles target to lymph node triggering enhanced innate and adaptive antitumor immunity. Biomaterials 130, 56-66 (2017).
Nakamura, T., Haloho, S. E. E. and Harashima, H. Intravenous liposomal vaccine enhances CTL generation, but not until antigen presentation. Journal of Controlled Release 343, 1-12 (2022).
Qin, H. et al. Development of a Cancer Vaccine Using In Vivo Click-Chemistry-Mediated Active Lymph Node Accumulation for Improved Immunotherapy. Advanced Materials 33, 2006007 (2021).
Roy, D. G. et al. Adjuvant oncolytic virotherapy for personalized anti-cancer vaccination. Nature Communications 12, 2626 (2021).
Kirkin, A. F., Dzhandzhugazyan, K. and Zeuthen, J. Melanoma-associated antigens recognized by cytotoxic T lymphocytes. APMIS 106, 665-679 (1998).
Pedersen, S. R., Sorensen, M. R., Buus, S., Christensen, J. P. and Thomsen, A. R. Comparison of Vaccine-Induced Effector CD8 T Cell Responses Directed against Self- and Non-Self-Tumor Antigens: Implications for Cancer Immunotherapy. The Journal of Immunology 191, 3955-3967 (2013).
Galon, J. and Bruni, D. Approaches to treat immune hot, altered and cold tumours with combination immunotherapies. Nature Reviews Drug Discovery 18, 197-218 (2019).
Saxena, M., van der Burg, S. H., Melief, C. J. M. and Bhardwaj, N. Therapeutic cancer vaccines. Nature Reviews Cancer 21, 360-378 (2021).
Mangraviti, A. et al. Polymeric nanoparticles for nonviral gene therapy extend brain tumor survival in vivo. ACS Nano 9, 1236-1249 (2015).
Shen, J. et al. Suprachoroidal gene transfer with nonviral nanoparticles. Sci Adv 6 (2020).
Shmueli, R. B., Sunshine, J. C., Xu, Z., Duh, E. J. and Green, J. J. Gene delivery nanoparticles specific for human microvasculature and macrovasculature. Nanomedicine 8, 1200-1207 (2012).
Zhang, Y.-N., Poon, W., Tavares, A. J., McGilvray, I. D. and Chan, W. C. W. Nanoparticle-liver interactions: Cellular uptake and hepatobiliary elimination. Journal of Controlled Release 240, 332-348 (2016).
Samaridou, E., Heyes, J. and Lutwyche, P. Lipid nanoparticles for nucleic acid delivery: Current perspectives. Advanced Drug Delivery Reviews 154-155, 37-63 (2020).
Wilhelm, S. et al. Analysis of nanoparticle delivery to tumours. Nature Reviews Materials 1, 16014 (2016).
Tacken, P. J., de Vries, I. J. M., Torensma, R. and Figdor, C. G. Dendritic-cell immunotherapy: from ex vivo loading to in vivo targeting. Nature Reviews Immunology 7, 790-802 (2007).
Phua, K. K. L. Towards Targeted Delivery Systems: Ligand Conjugation Strategies for mRNA Nanoparticle Tumor Vaccines. Journal of Immunology Research 2015, 680620 (2015).
Pozzi, L.-A. M., Maciaszek, J. W. and Rock, K. L. Both Dendritic Cells and Macrophages Can Stimulate Naive CD8 T Cells In Vivo to Proliferate, Develop Effector Function, and Differentiate into Memory Cells. The Journal of Immunology 175, 2071-2081 (2005).
Karlsson, J. et al. Photocrosslinked Bioreducible Polymeric Nanoparticles for Enhanced Systemic siRNA Delivery as Cancer Therapy. Advanced Functional Materials 31, 2009768 (2021b).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising a polymer of formula (I) and one or more nucleic acids encoding antigen: 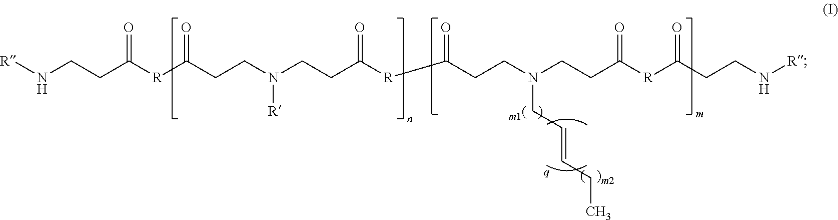
wherein:
m and n are each independently an integer from 1 to 10,000;
m1 is an integer selected from 0, 1, 2, 3, 4, 5, 6, 7, 8, 9, and 10;
m2 is an integer selected from the group consisting of 1, 2, 3, 4, 5, 6, 7, 8, 9, 10, 11, 12, 13, 14, 15, 16, 17, 18, 19, and 20;
q is an integer selected from 0 or 1;
wherein —(CH₂)ₘ₁—(C═C)_{q}—(CH₂)ₘ₂—CH₃ comprises a hydrophobic sidechain;
R comprises a divalent radical comprising a biodegradable ester linkage and/or a bioreducible disulfide linkage;
R′ is hydrophilic sidechain comprising a monovalent radical derived from a hydrophilic amine monomer;
R″ is monovalent radical derived from an amine-containing end capping group; and
pharmaceutically acceptable salts thereof.

2. The composition of claim 1, wherein:
a)R is selected from the group consisting of: 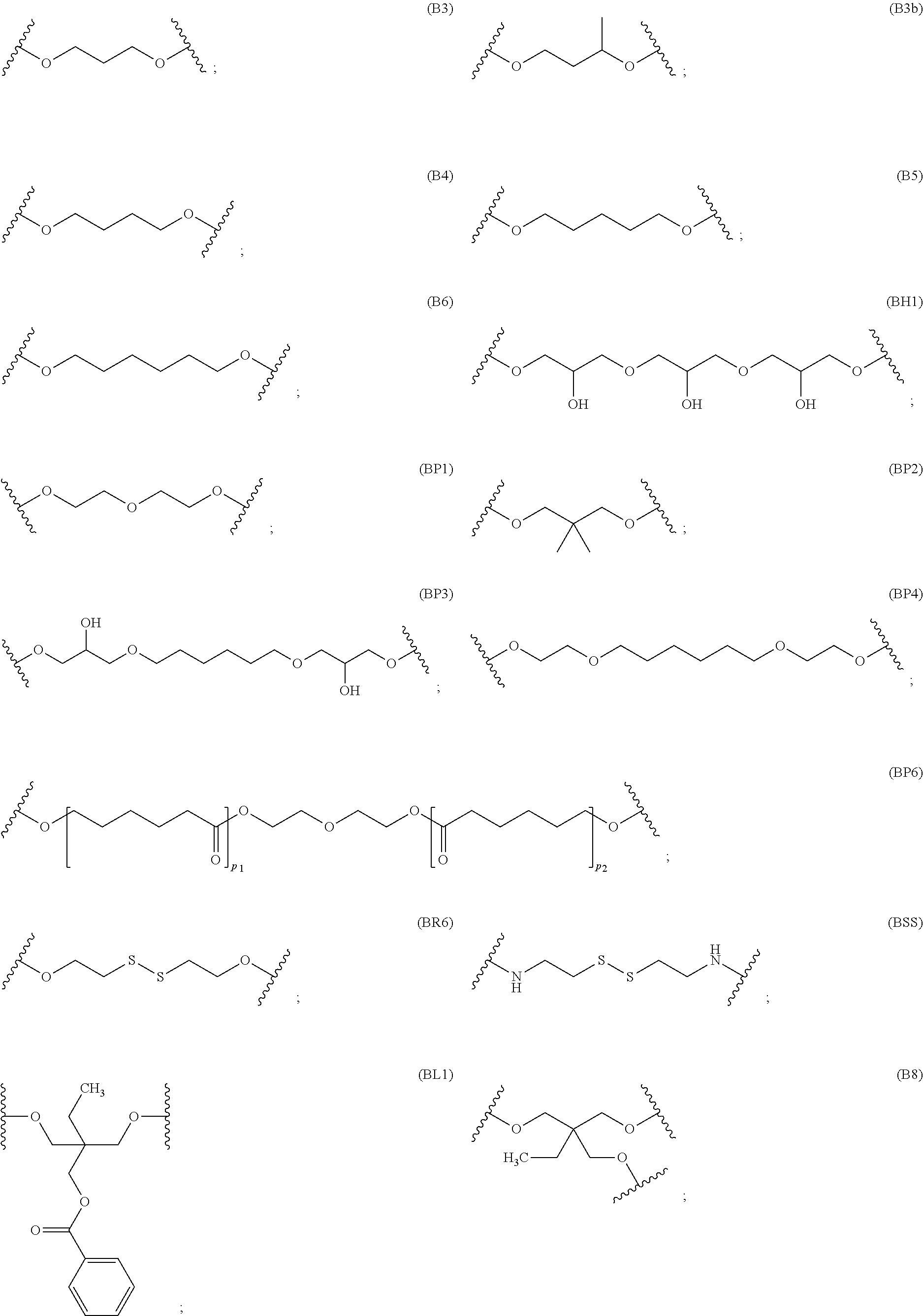 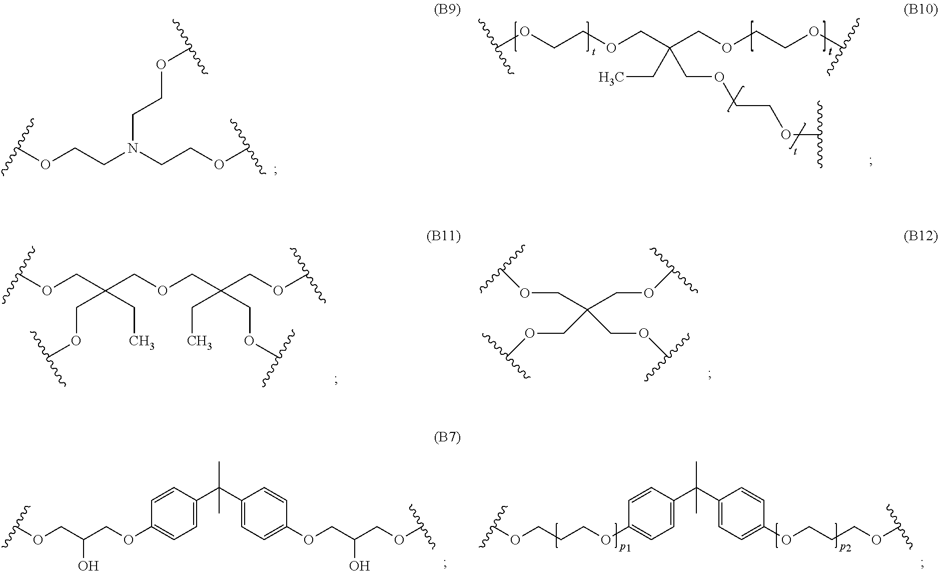
wherein each p1, p2, and t is independently an integer selected from the group consisting of 1, 2, 3, 4, 5, 6, 7, 8, 9, and 10;
(b) R′ is selected from the group consisting of: 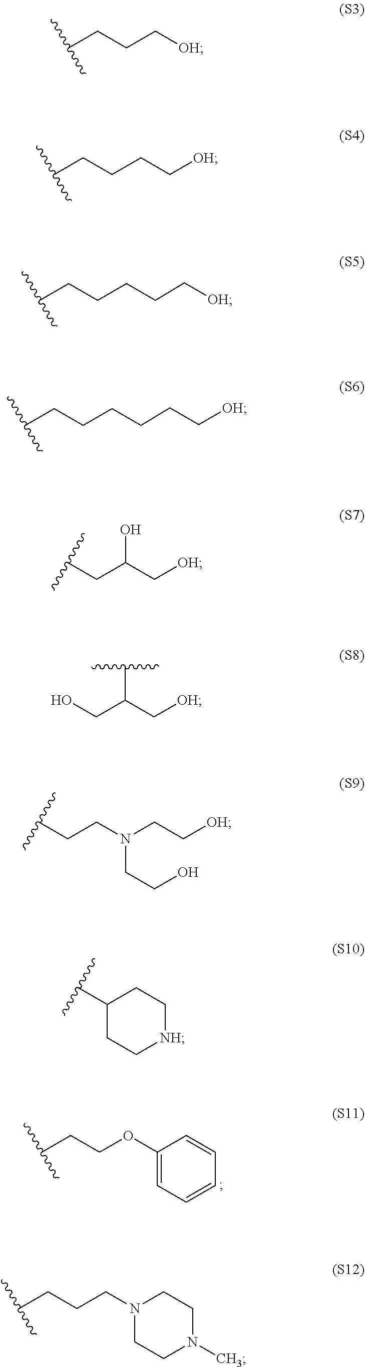 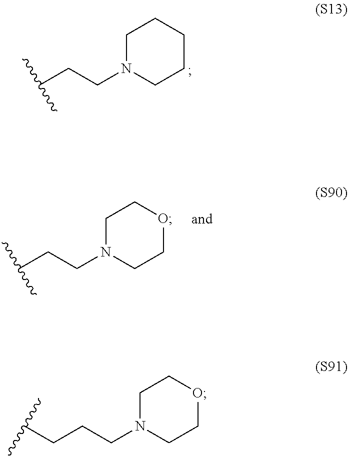 [SMILES: *CCN1CCCCC1 ; *CCN1CCOCC1 ; "and" ; *CCCN1CCOCC1]
(c) R″ is selected from the group consisting of: 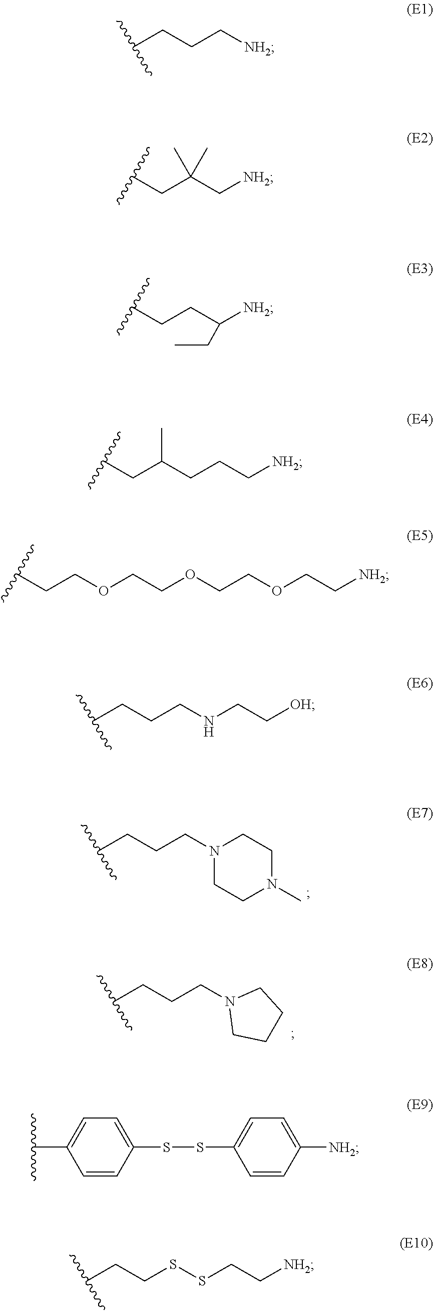 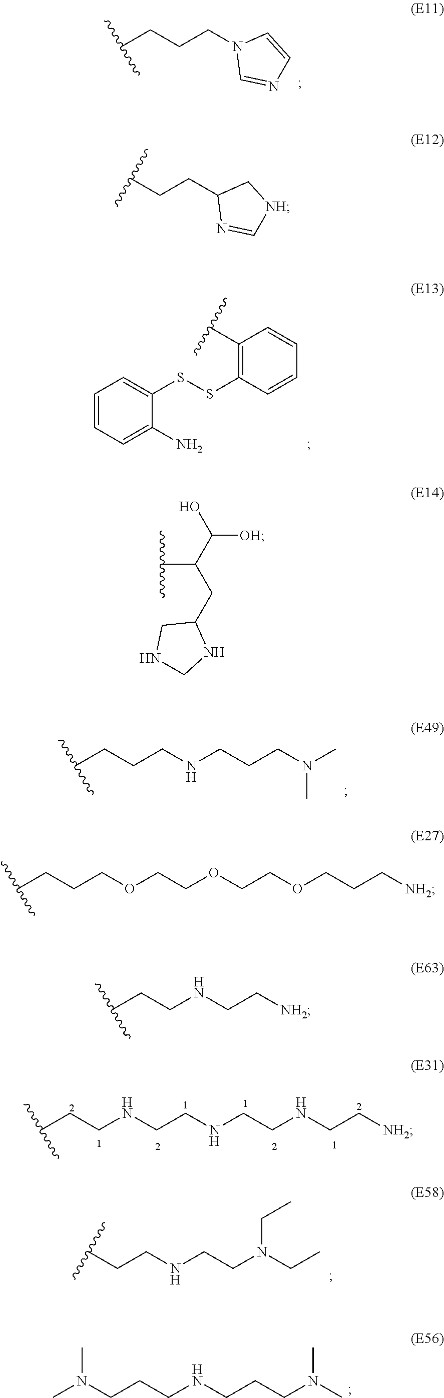 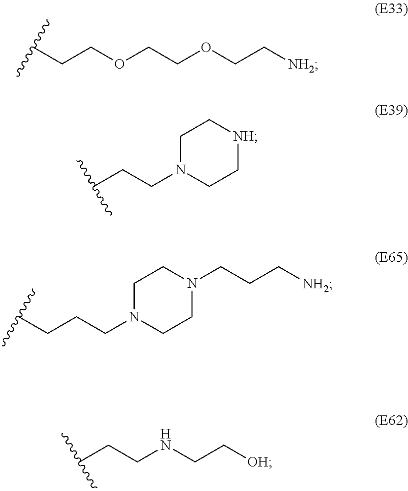 [SMILES: *CCOCCOCCN ; *CCN1CCNCC1 ; *CCCN1CCN(CCCN)CC1 ; *CCNCCO] 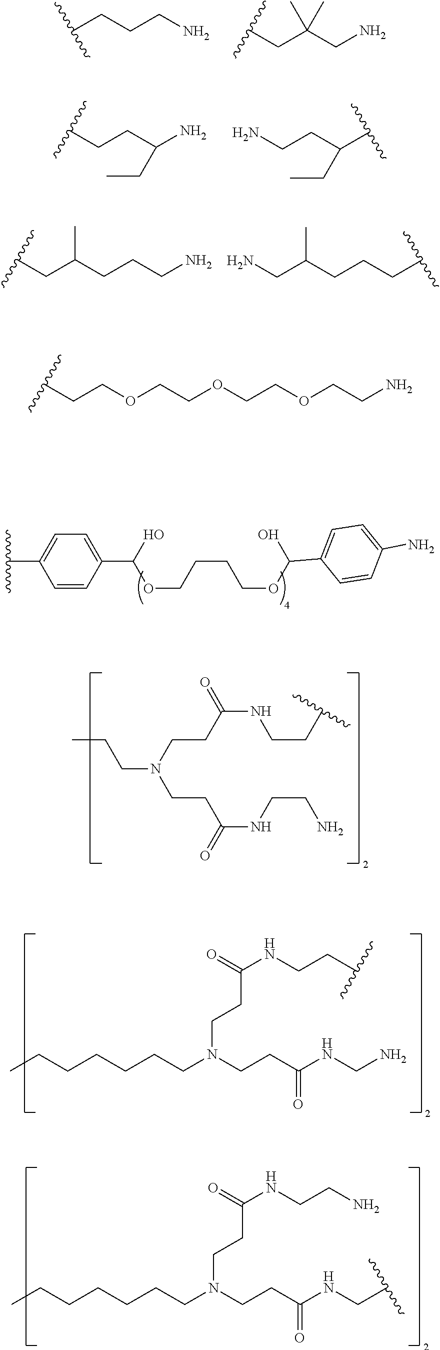 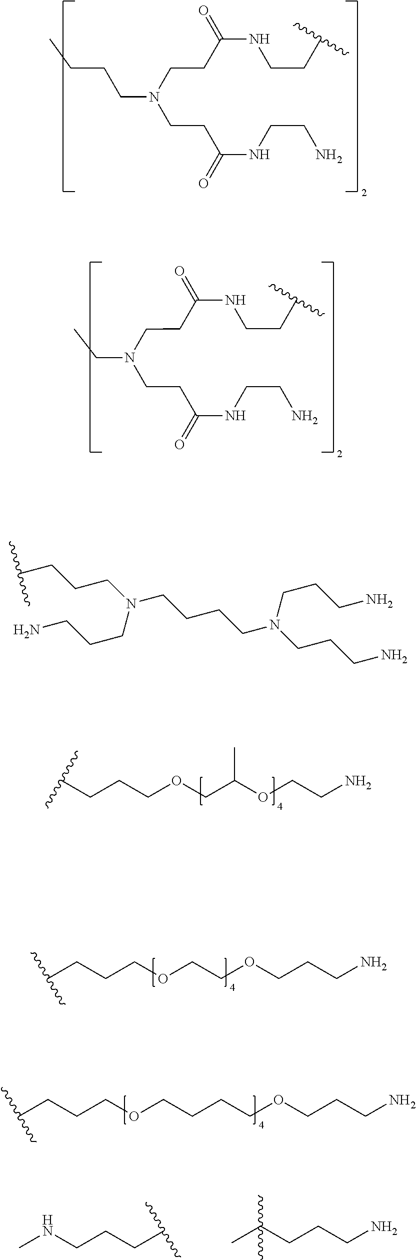 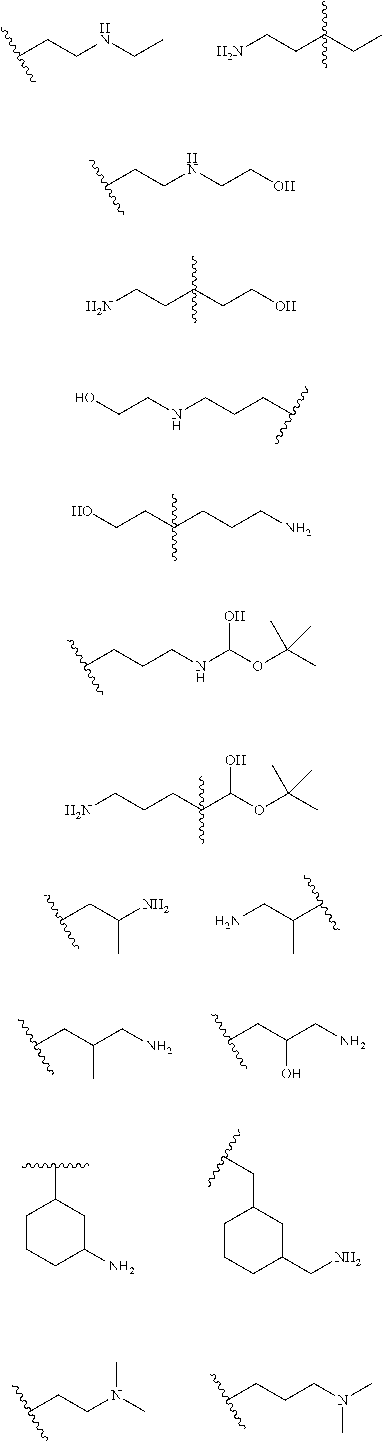 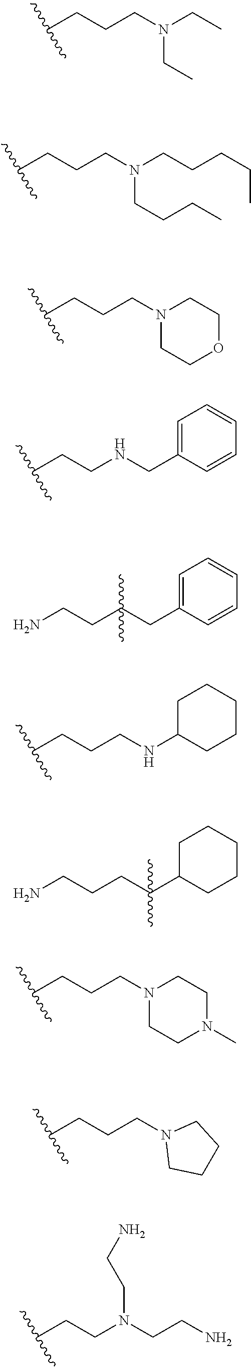 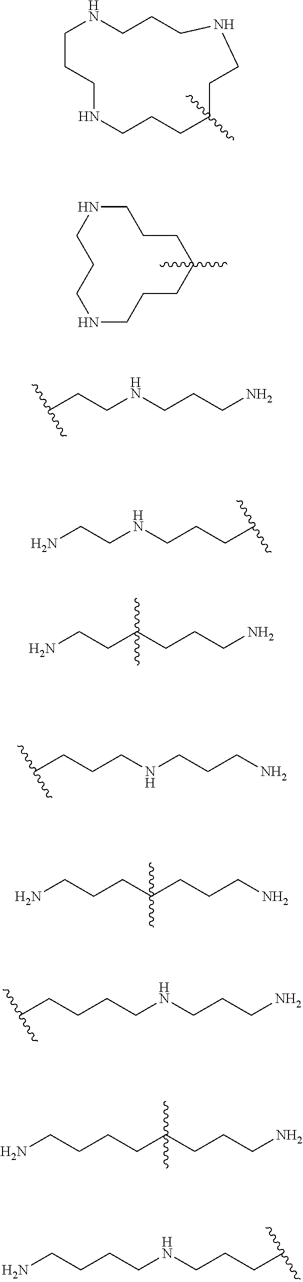 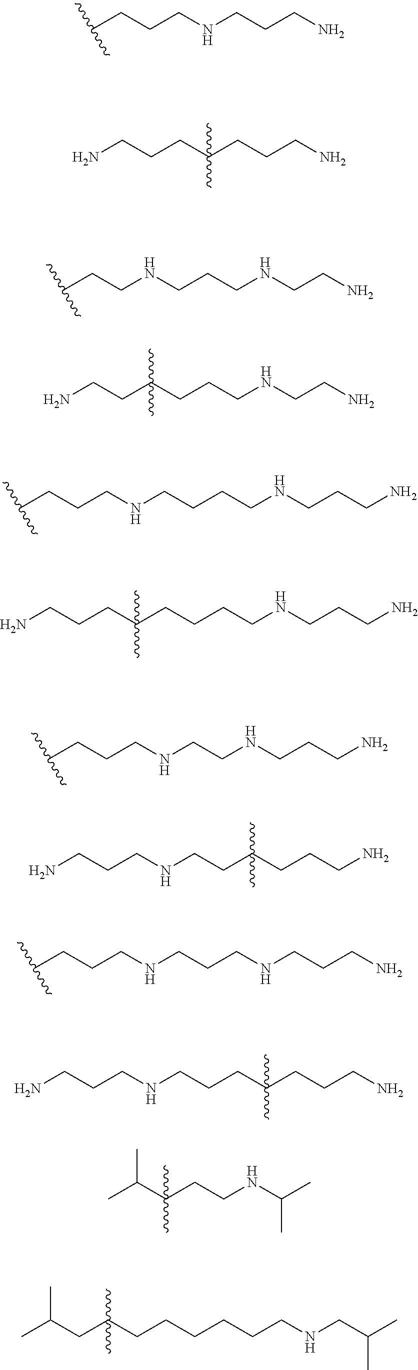 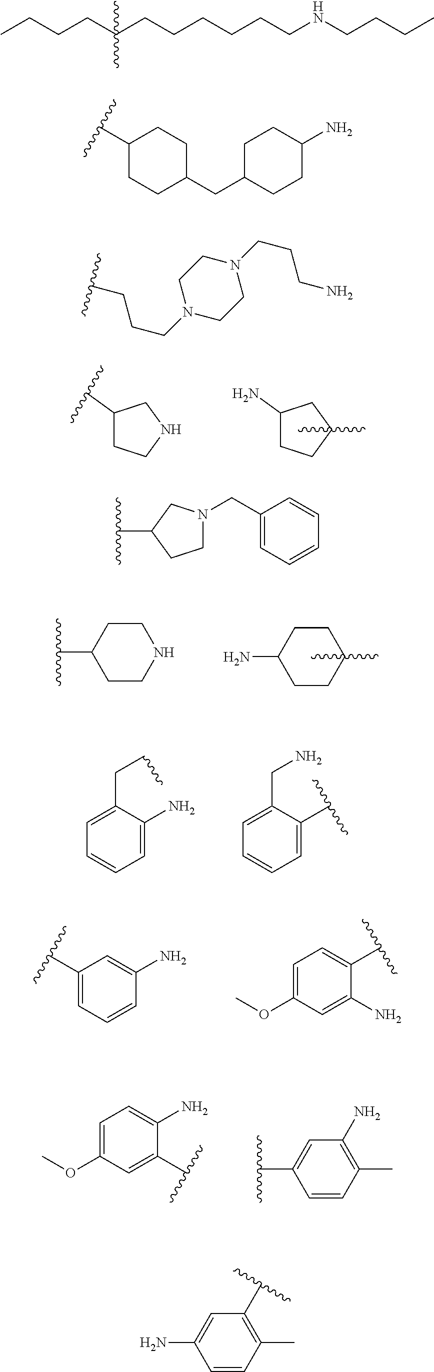 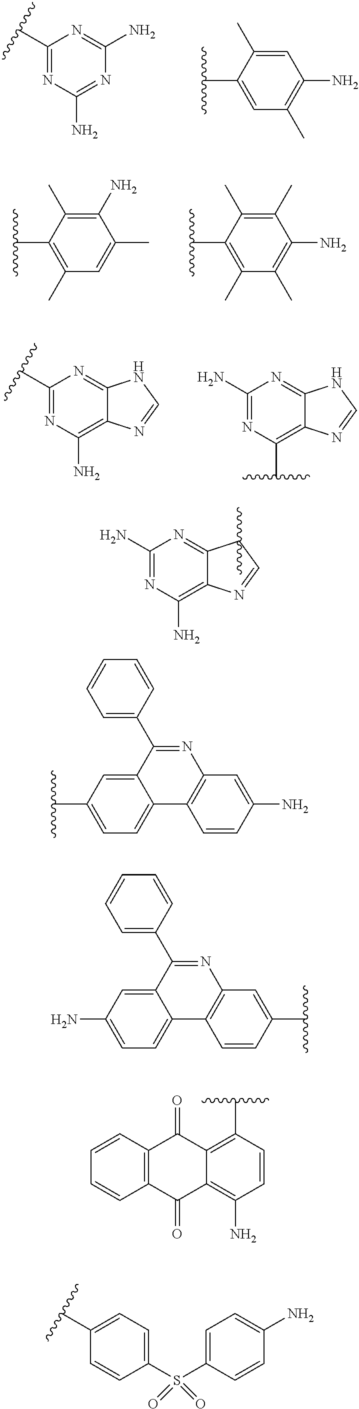 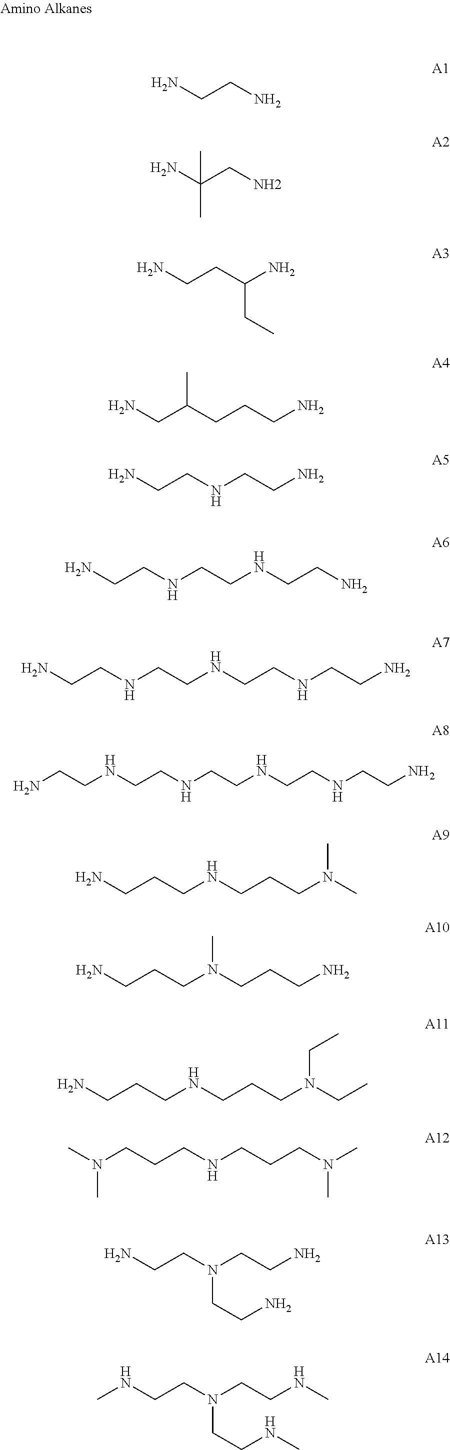 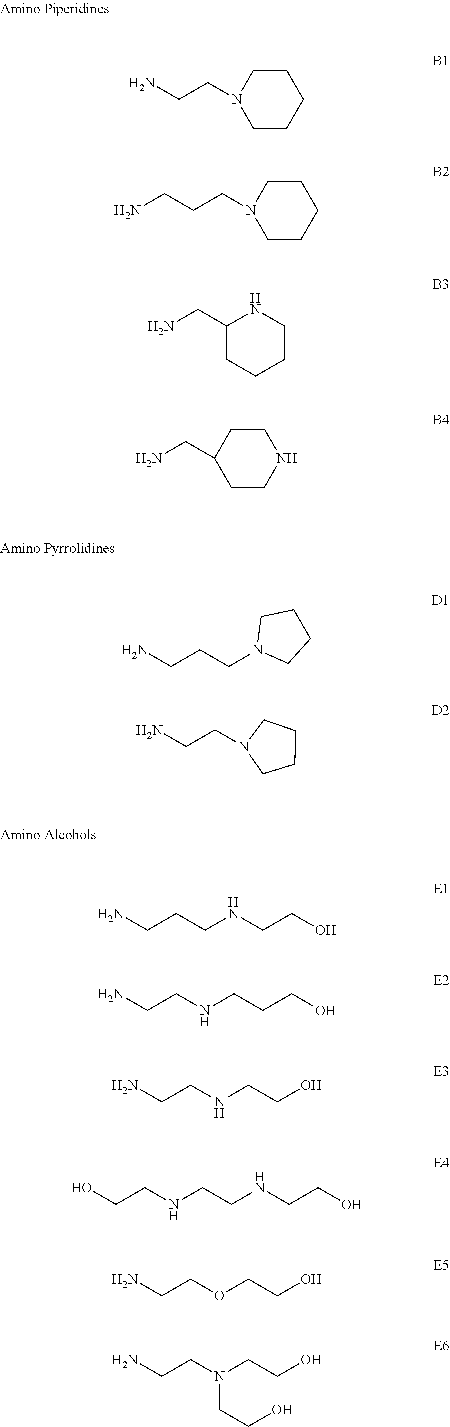 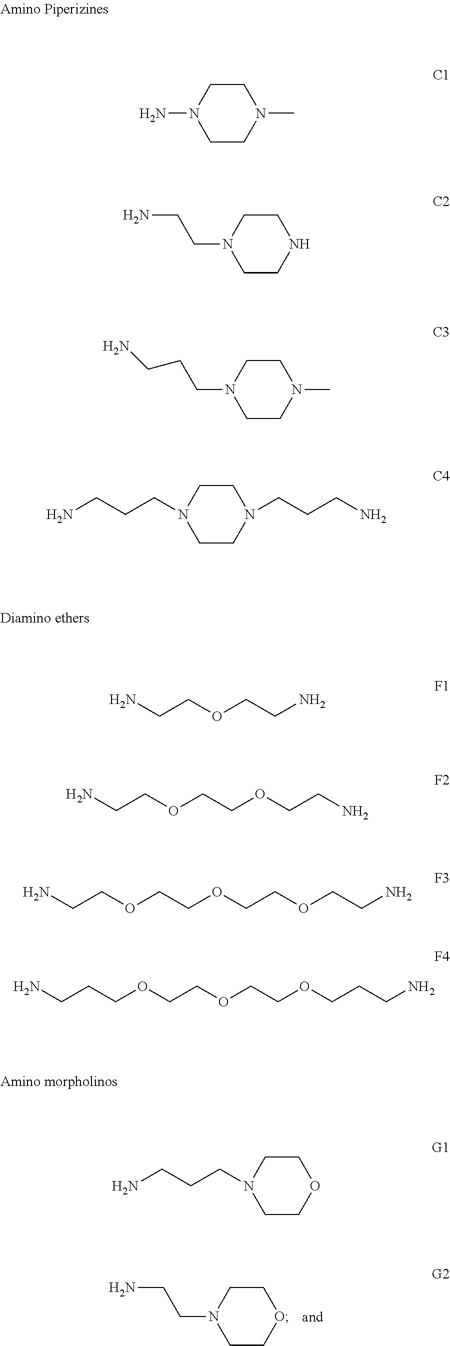 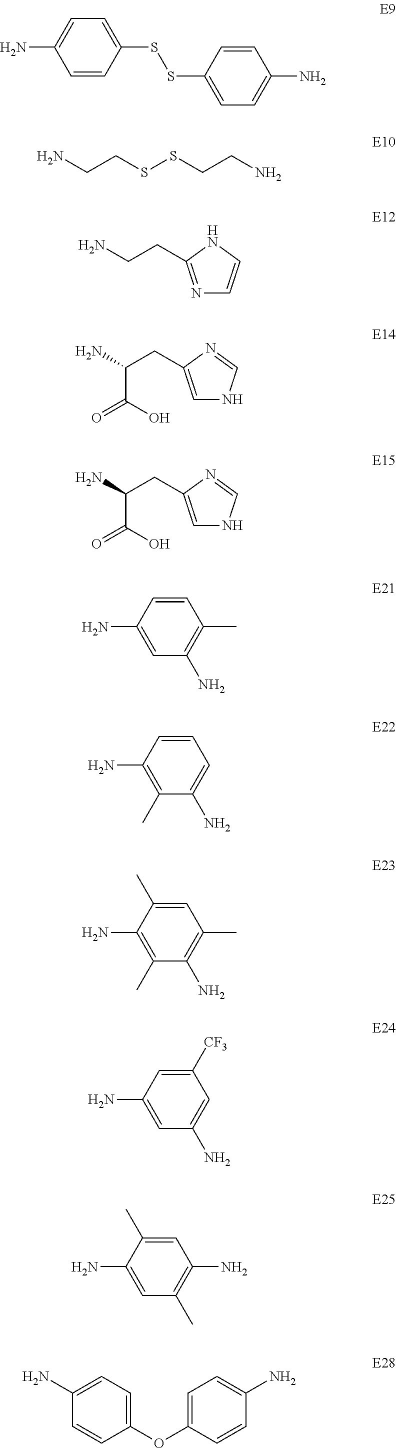 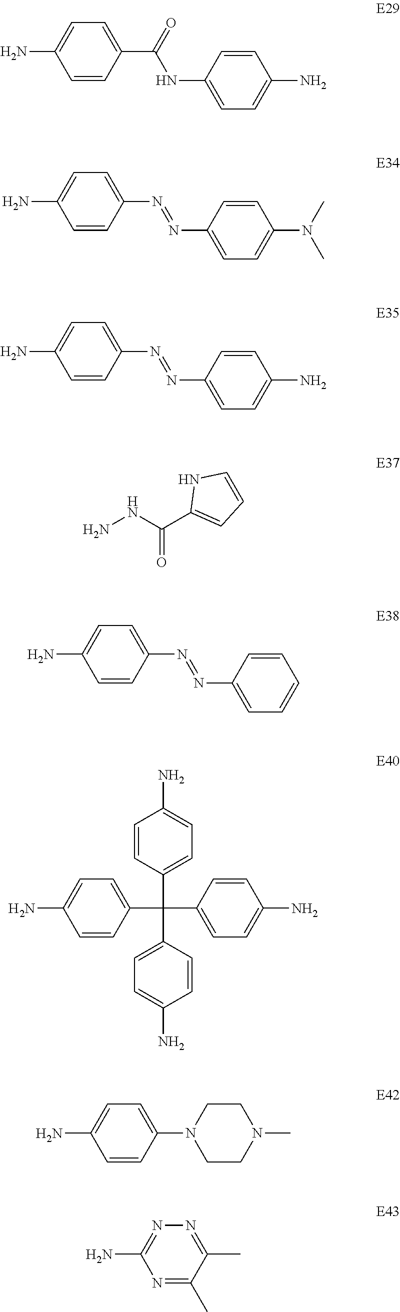 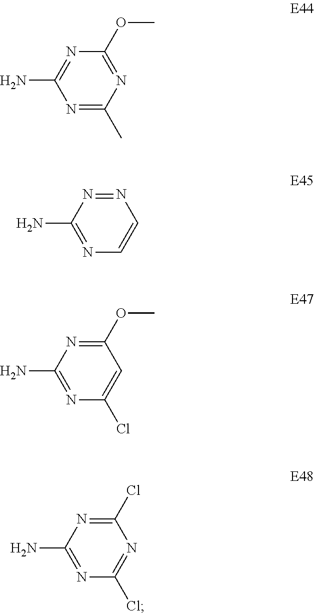
(d) —(CH₂)ₘ₁—(C═C)_{q}—(CH₂)ₘ₂—CH₃ is selected from the group consisting of:

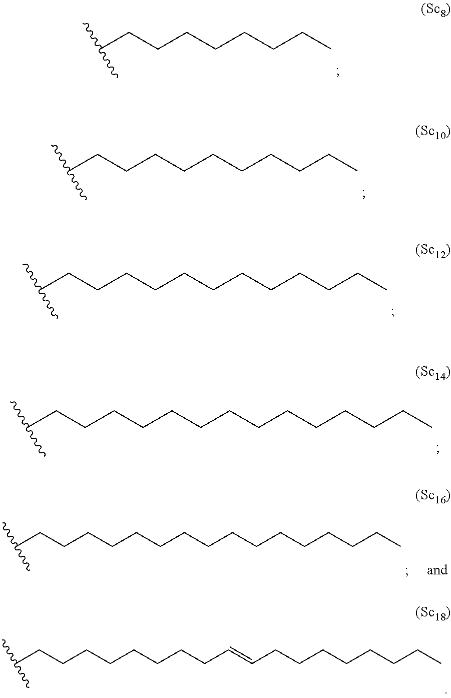

3-5. (canceled)

6. The composition of claim 2, wherein:
(a) R is: 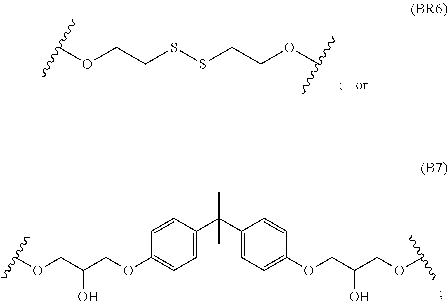 [SMILES: *OCCSSCCO* ; *OCC(O)COc1ccc(C(C)(C)c2ccc(OCC(O)CO*)cc2)cc1]
(b) R′ is: 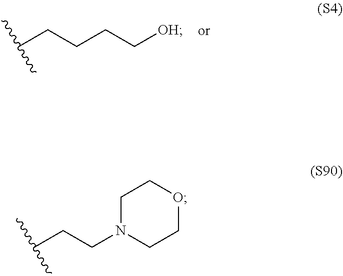 [SMILES: *CCCCO ; *CCN1CCOCC1]
(c) R″ is selected from the group consisting of: 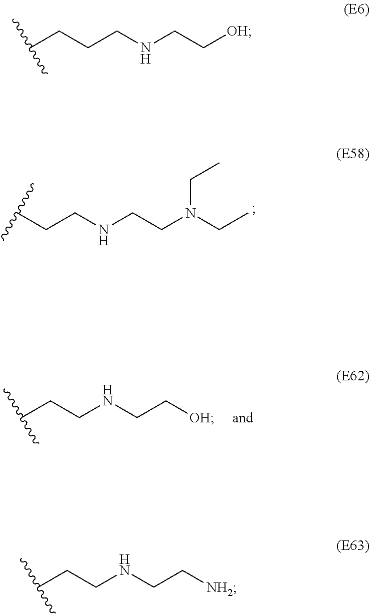 [SMILES: *CCCNCCO ; *CCNCCN(CC)CC ; *CCNCCO ; "and" ; *CCNCCN]
and
(d) —(CH₂)ₘ₁—(C═C)_{q}—(CH₂)ₘ₂—CH₃ is selected from the group consisting of Sc12, Sc16, and Sc18.

7-9. (canceled)

10. The composition of claim 1, wherein the compound of formula (I) is selected from: 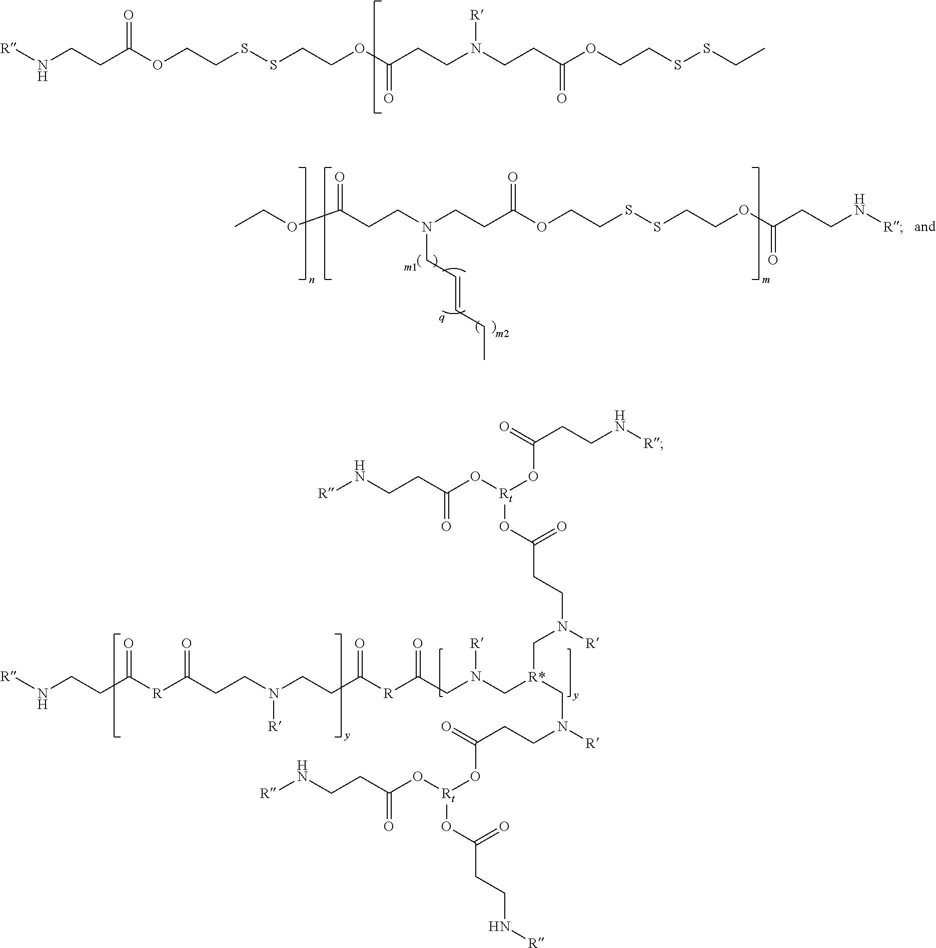
wherein each R* is a triacrylate, quanternary, or hexafunctional acrylate monomer selected from the group consisting of: 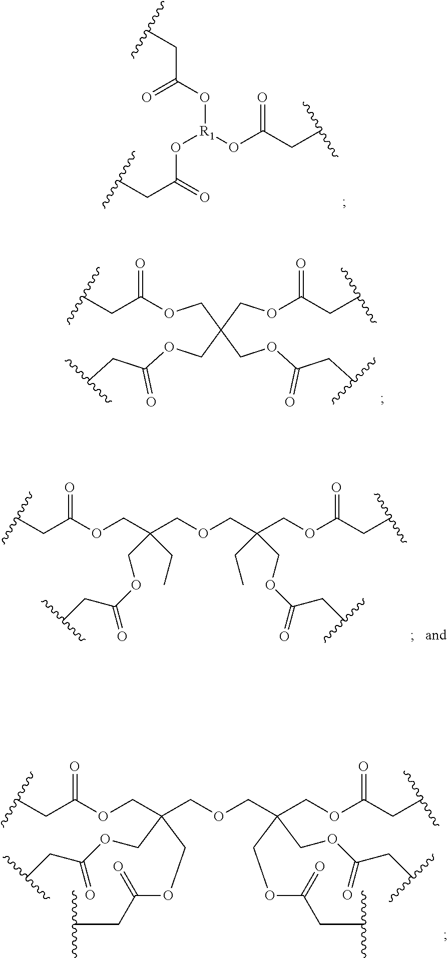
wherein each Rr is independently a trivalent group; and each y is independently an integer from 1 to 10,000.

11. (canceled)

12. The composition of claim 1, wherein a ratio of the hydrophobic side chain to the hydrophilic side chain is between about 10:90 hydrophobic side chain:hydrophilic side chain to about 90:10 hydrophobic side chain:hydrophilic side chain.

13. The composition of claim 1, wherein the composition is selected from the group consisting of:
(a)BR6-S4, Sc16-E6, 50%/50% ratio of S4/Sc16;
BR6-S4, Sc16-E62, 50%/50% ratio of S4/Sc16;
BR6-S4, Sc16-E63, 50%/50% ratio of S4/Sc16;
BR6-S4, Sc18-E6, 50%/50% ratio of S4/Sc18;
BR6-S4, Sc18-E62, 50%/50% ratio of S4/Sc18; and
BR6-S4, Sc18-E63, 50%/50% ratio of S4/Sc18, or
(b) B7-S90, Sc12-E6, 50%/50% ratio of S90/Sc12;
B7-S90, Sc12-E6, 20%/80% ratio of S90/Sc12;
B7-S90, Sc12-E58, 50%/50% ratio of S90/Sc12;
B7-S90, Sc12-E58, 20%/80% ratio of S90/Sc12;
B7-S90, Sc12-E63, 50%/50% ratio of S90/Sc12; and
B7-S90, Sc12-E63, 20%/80% ratio of S90/Sc12.

14. (canceled)

15. The composition of claim 1, wherein the composition comprises a weight ratio between the polymer and the nucleic acid having a range from between about 30 w/w and about 200 w/w or between about 50 w/w and about 150 w/w.

16. (canceled)

17. The composition of claim 1, wherein the one or more nucleic acids are selected from:
(a) DNA or RNA;
(b) an oligonucleotide, a cyclic dinucleotide, plasmid DNA, linear DNA, siRNA, miRNA, mRNA, and combinations thereof; or
(c) self-amplifying mRNA (SAM).

18-20. (canceled)

21. The composition of claim 1, further comprising one or more immunomodulatory nucleic acids.

22. The composition of claim 21, wherein the one or more immunomodulatory nucleic acids are selected from the group consisting of CpG, GpG, poly(I:C), and a cyclic dinucleotide (CDN).

23. The composition of claim 1, comprising a nanoparticle comprising a compound of formula (I) and:
(a) one or more nucleic acids;
(b) encapsulating an mRNA that encodes an autoreactive antigen, wherein the autoreactive antigen comprises myelin oligodendrocyte glycoprotein (MOG).
(c) GpG; and/or
(d) rapamycin.

24-27. (canceled)

28. The composition of claim 1, further comprising lipid-PEG: wherein:
(a) the lipid-PEG is admixed with the compound of formula (I) at a varying mass percent;
(b) the lipid-PEG is selected from the group consisting of 1,2-dimyristoyl-rac-glycero-3-methoxypolyethylene glycol 2000 (DMG-PEG2k) and C18-PEG2k;
(c) the composition comprises DMG-PEG2k at a weight percent from about 2 wt % to about 10 wt %; and/or
(d) a zeta-potential of the nanoparticle varies with a varying mass percent of lipid-PEG.

29-33. (canceled)

34. The composition of claim 1, further comprising one or more excipients;
wherein the one or more excipients include one or more cryoprotectants, one or more sugars or sugar alcohols, MgCl₂, and combinations thereof;
wherein the one or more cryoprotectants comprise a sugar selected from the group consisting of glucose, fructose, sorbitol, mannitol, sucrose, trehalose, and raffinose; and
wherein the one or more sugar alcohols comprise sorbitol.

35-38. (canceled)

39. The composition of claim 34, wherein the composition is lyophilized; and/or comprises a storable powder.

40. (canceled)

41. A genetic vaccine comprising a composition of claim 1 and one or more nucleic acids, wherein the genetic vaccine targets one or more antigen presenting cells.

42. The genetic vaccine of claim 41, wherein the one or more nucleic acids comprises an mRNA encoding one or more antigens and/or one or more immunomodulatory nucleic acids selected from the group consisting of CpG, GpG, poly(I:C), and cyclic dinucleotides (CDN).

43. A method for delivering one or more nucleic acids to a subject, the method comprising administering a composition of claim 1 to the subject, wherein the administering comprises systemically administering or intravenously administering the composition or the genetic vaccine to the subject.

44-45. (canceled)

46. A method for treating a disease or condition, the method comprising administering a composition of claim 1 to a subject in need of treatment thereof, wherein the disease or condition is selected from the group consisting of a cancer, an infectious disease, and an autoimmune disease.

47. (canceled)

48. The method of claim 47, wherein:
(a) the infectious disease is selected from the group consisting of a coronavirus, influenza, and rabies;
(b) the cancer comprises a solid tumor in one or more organs selected from the group consisting of the brain, colon, breast, prostate, liver, kidney, lung, esophagus, head and neck, ovaries, cervix, stomach, colon, rectum, bladder, uterus, testes, and pancreas;
(c) the cancer comprises a metastatic cancer selected from the following types of cancer and metastasis sites:
bladder: bone, liver, lung;
breast: bone, brain, liver, lung;
colon: liver, lung, peritoneum;
kidney: adrenal gland, bone, brain, liver, lung;
lung: adrenal gland, bone, brain, liver, other lung;
melanoma: bone, brain, liver, lung, skin, muscle;
ovary: liver, lung, peritoneum;
pancreas: liver, lung, peritoneum;
prostate: adrenal gland, bone, liver, lung;
rectal: liver, lung, peritoneum;
stomach: liver, lung, peritoneum;
thyroid: bone, liver, lung; and
uterus: bone, liver, lung, peritoneum, vagina;
(d) the autoimmune disease is selected from the group consisting of type I diabetes mellitus (TID), Crohn's disease, ulcerative colitis, myasthenia gravis, vitiligo, Graves' disease, Hashimoto's disease, Addison's disease and autoimmune gastritis, autoimmune hepatitis, primary biliary cirrhosis, autoimmune thrombocytopenia, rheumatoid arthritis, systemic lupus erythematosus, progressive systemic sclerosis and variants, polymyositis and dermatomyositis, inflammatory bowel disease, celiac disease, inflammatory myositis, Sjogren's syndrome, multiple sclerosis, psoriasis and scleroderma; and
wherein the subject is a human or an animal and the treatment method is selected from a prophylactic treatment method, a therapeutic treatment method, and combinations thereof.

49-53. (canceled)

54. A kit comprising a composition of claim 1, the kit comprising one or more of: one or more compounds of formula (I), one or more nucleic acids, reagents, and instructions for use.
